SEQUENCE LISTING (303 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
RASQSVSSYLA

SEQ ID NO: 12 - protein, 7 aa, synthetic construct
DASNRAT

SEQ ID NO: 13 - protein, 9 aa, synthetic construct
QQSSNWPRT

SEQ ID NO: 14 - protein, 107 aa, synthetic construct [sequence omitted]

SEQ ID NO: 15 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
NSGMH

SEQ ID NO: 17 - protein, 17 aa, synthetic construct
VIWYDGSKRYYADSVKG

SEQ ID NO: 18 - protein, 4 aa, synthetic construct
NDDY

SEQ ID NO: 19 - protein, 113 aa, synthetic construct [sequence omitted]

SEQ ID NO: 20 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, synthetic construct
RASKSVSTSGFSYLH

SEQ ID NO: 22 - protein, 7 aa, synthetic construct
LASNLES

SEQ ID NO: 23 - protein, 9 aa, synthetic construct
QHSWELPLT

SEQ ID NO: 24 - protein, 5 aa, synthetic construct
SYYLY

SEQ ID NO: 25 - protein, 17 aa, synthetic construct
GVNPSNGGTNFSEKFKS

SEQ ID NO: 26 - protein, 11 aa, synthetic construct
RDSNYDGGFDY

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 111 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, synthetic construct; identical to SEQ ID NO: 10

SEQ ID NO: 32 - protein, 218 aa, synthetic construct; identical to SEQ ID NO: 5

SEQ ID NO: 33 - protein, 218 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, synthetic construct
SDYWG

SEQ ID NO: 36 - protein, 16 aa, synthetic construct
FITYSGSTSYNPSLKS

SEQ ID NO: 37 - protein, 19 aa, synthetic construct
MPSFITLASLSTWEGYFDF

SEQ ID NO: 38 - protein, 11 aa, synthetic construct
KASQSIHKNLA

SEQ ID NO: 39 - protein, 7 aa, synthetic construct
YANSLQT

SEQ ID NO: 40 - protein, 8 aa, synthetic construct
QQYYSGWT

SEQ ID NO: 41 - protein, 127 aa, synthetic construct
EVQLQESGPGLVKPSQSLSLTCSVTGSSIASDYWGWIRKFPGNKMEWMGFITYSGSTSYNPSLKSRISITRDTSKNQFFLQLHSVTTDDTATYSCARMPSFITLASLSTWEGYFDFWGPGTMVTVSS

SEQ ID NO: 42 - protein, 106 aa, synthetic construct
DIQMTQSPSLLSASVGDRVTLNCKASQSIHKNLAWYQQKLGEAPKFLIYYANSLQTGIPSRFSGSGSGTDFTLTISGLQPEDVATYFCQQYYSGWTFGGGTKVELK

SEQ ID NO: 43 - protein, 127 aa, synthetic construct
EVQLQESGPGLVKPSETLSLTCTVSGGSISSDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 44 - protein, 127 aa, synthetic construct
EVQLQESGPGLVKPSETLSLTCTVSGGSISSDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 45 - protein, 127 aa, synthetic construct
EVQLQESGPGLVKPSETLSLTCTVSGGSISSDYWGWIRQPPGKGLEWIGFITYSGSTSYNPSLKSRITISRDTSKNQFSLKLSSVTAADTAVYYCARMPSFITLASLSTWEGYFDFWGQGTMVTVSS

SEQ ID NO: 46 - protein, 127 aa, synthetic construct
EVQLQESGPGLVKPSETLSLTCTVSGSSISSDYWGWIRQPPGKGLEWMGFITYSGSTSYNPSLKSRITISRDTSKNQFSLKLSSVTAADTAVYYCARMPSFITLASLSTWEGYFDFWGQGTMVTVSS

SEQ ID NO: 47 - protein, 127 aa, synthetic construct
EVQLQESGPGLVKPSETLSLTCTVSGGSISSDYWGWIRQPPGKGLEWIGFITYSGSTSYNPSLKSRVTISRDTSKNQFSLKLHSVTAADTAVYYCARMPSFITLASLSTWEGYFDFWGQGTMVTVSS

SEQ ID NO: 48 - protein, 127 aa, synthetic construct
EVQLQESGPGLVKPSETLSLTCTVSGGSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 49 - protein, 127 aa, synthetic construct
EVQLQESGPGLVKPSETLSLTCTVSGSSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 50 - protein, 127 aa, synthetic construct
EVQLQESGPGLVKPSETLSLTCTVSGSSISSDYWGWIRQPPGKGLEWMGFITYSGSTSYNPSLKSRITISVDTSKNQFSLKLHSVTAADTAVYYCARMPSFITLASLSTWEGYFDFWGQGTMVTVSS

SEQ ID NO: 51 - protein, 127 aa, synthetic construct
EVQLQESGPGLVKPSETLSLTCAVSGYSISSDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 52 - protein, 127 aa, synthetic construct
EVQLQESGPGLVKPSETLSLTCAVSGYSISSDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 53 - protein, 127 aa, synthetic construct
EVQLQESGPGLVKPSETLSLTCAVSGYSISSDYWGWIRQPPGKGLEWIGFITYSGSTSYNPSLKSRITISRDTSKNQFSLKLSSVTAADTAVYYCARMPSFITLASLSTWEGYFDFWGQGTMVTVSS

SEQ ID NO: 54 - protein, 127 aa, synthetic construct
EVQLQESGPGLVKPSETLSLTCAVSGSSISSDYWGWIRQPPGKGLEWMGFITYSGSTSYNPSLKSRITISRDTSKNQFSLKLSSVTAADTAVYYCARMPSFITLASLSTWEGYFDFWGQGTMVTVSS

SEQ ID NO: 55 - protein, 127 aa, synthetic construct
EVQLQESGPGLVKPSETLSLTCAVSGYSISSDYWGWIRQPPGKGLEWIGFITYSGSTSYNPSLKSRVTISRDTSKNQFSLKLHSVTAADTAVYYCARMPSFITLASLSTWEGYFDFWGQGTMVTVSS

SEQ ID NO: 56 - protein, 127 aa, synthetic construct
EVQLQESGPGLVKPSETLSLTCAVSGYSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 57 - protein, 127 aa, synthetic construct
EVQLQESGPGLVKPSETLSLTCAVSGSSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 58 - protein, 127 aa, synthetic construct
EVQLQESGPGLVKPSETLSLTCAVSGSSISSDYWGWIRQPPGKGLEWMGFITYSGSTSYNPSLKSRITISRDTSKNQFSLKLHSVTAADTAVYYCARMPSFITLASLSTWEGYFDFWGQGTMVTVSS

SEQ ID NO: 59 - protein, 106 aa, synthetic construct
DIQMTQSPSSLSASVGDRVTITCKASQSIHKNLAWYQQKPGKAPKLLIYYANSLQTGVPSRFSGSGSGTDFTLTISSLQPEDFATYYCQQYYSGWTFGGGTKVEIK

SEQ ID NO: 60 - protein, 106 aa, synthetic construct
DIQMTQSPSSLSASVGDRVTITCKASQSIHKNLAWYQQKPGKAPKFLIYYANSLQTGVPSRFSGSGSGTDFTLTISSLQPEDFATYYCQQYYSGWTFGGGTKVEIK

SEQ ID NO: 61 - protein, 106 aa, synthetic construct
DIQMTQSPSSLSASVGDRVTITCKASQSIHKNLAWYQQKPGKAPKFLIYYANSLQTGIPSRFSGSGSGTDFTLTISSLQPEDFATYYCQQYYSGWTFGGGTKVEIK

SEQ ID NO: 62 - protein, 106 aa, synthetic construct
DIQMTQSPSSLSASVGDRVTITCKASQSIHKNLAWYQQKPGKVPKLLIYYANSLQTGVPSRFSGSGSGTDFTLTISSLQPEDVATYYCQQYYSGWTFGGGTKVEIK

SEQ ID NO: 63 - protein, 106 aa, synthetic construct
DIQMTQSPSSLSASVGDRVTITCKASQSIHKNLAWYQQKPGKVPKFLIYYANSLQTGVPSRFSGSGSGTDFTLTISSLQPEDVATYYCQQYYSGWTFGGGTKVEIK

SEQ ID NO: 64 - protein, 106 aa, synthetic construct
DIQMTQSPSSLSASVGDRVTITCKASQSIHKNLAWYQQKPGKVPKFLIYYANSLQTGIPSRFSGSGSGTDFTLTISSLQPEDVATYYCQQYYSGWTFGGGTKVEIK

SEQ ID NO: 65 - protein, 129 aa, synthetic construct
EVQLQESGPGLVKPSETLSLTCTVSGSSIASDYWGWIRQPPGKGLEWIGFITYSGSTSYNPSLKSRVTISVDTSKNQFSLKLSSVTAADTAVYYCARMPSFITLASLSTWEGYFDFWGQGTMVTVSSAS

SEQ ID NO: 66 - protein, 127 aa, synthetic construct
EVQLQESGPGLVKPSETLSLTCTVSGSSIASDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 67 - protein, 127 aa, synthetic construct
EVQLQESGPGLVKPSETLSLTCTVSGSSIASDYWGWIRKPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 68 - protein, 127 aa, synthetic construct
EVQLQESGPGLVKPSETLSLTCTVSGSSIASDYWGWIRQPPGKKLEWIGFITYSGSTSYNPSLKSRVTISRDTSKNQFSLKLSSVTAADTAVYYCARMPSFITLASLSTWEGYFDFWGQGTMVTVSS

SEQ ID NO: 69 - protein, 127 aa, synthetic construct
EVQLQESGPGLVKPSETLSLTCTVSGSSIASDYWGWIRQPPGKGMEWIGFITYSGSTSYNPSLKSRVTISRDTSKNQFSLKLSSVTAADTAVYYCARMPSFITLASLSTWEGYFDFWGQGTMVTVSS

SEQ ID NO: 70 - protein, 127 aa, synthetic construct
EVQLQESGPGLVKPSETLSLTCTVSGSSIASDYWGWIRKPPGKKMEWIGFITYSGSTSYNPSLKSRVTISRDTSKNQFSLKLSSVTAADTAVYYCARMPSFITLASLSTWEGYFDFWGQGTMVTVSS

SEQ ID NO: 71 - protein, 127 aa, synthetic construct
EVQLQESGPGLVKPSETLSLTCTVSGSSIASDYWGWIRQFPGKGLEWIGFITYSGSTSYNPSLKSRVTISRDTSKNQFSLKLSSVTADDTAVYYCARMPSFITLASLSTWEGYFDFWGQGTMVTVSS

SEQ ID NO: 72 - protein, 127 aa, synthetic construct
EVQLQESGPGLVKPSETLSLTCTVSGSSIASDYWGWIRKPPGKKMEWIGFITYSGSTSYNPSLKSRVTISVDTSKNQFSLKLSSVTAADTAVYYCARMPSFITLASLSTWEGYFDFWGQGTMVTVSS

SEQ ID NO: 73 - protein, 127 aa, synthetic construct
EVQLQESGPGLVKPSETLSLTCSVTGSSIASDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 74 - protein, 127 aa, synthetic construct
EVQLQQSGAGLLKPSETLSLTCSVTGSSIASDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 75 - protein, 127 aa, synthetic construct
EVQLQESGPGLVKPPGTLSLTCSVTGSSIASDYWGWVRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 76 - protein, 106 aa, synthetic construct; identical to SEQ ID NO: 59

SEQ ID NO: 77 - protein, 106 aa, synthetic construct; identical to SEQ ID NO: 60

SEQ ID NO: 78 - protein, 106 aa, synthetic construct
DIQMTQSPSSLSASVGDRVTITCKASQSIHKNLAWYQQKPGKAPKLLIYYANSLQTGVPSRFSGSGSGTDFTLTISSLQPEDFATYFCQQYYSGWTFGGGTKVEIK

SEQ ID NO: 79 - protein, 106 aa, synthetic construct
DIQMTQSPSSLSASVGDRVTITCKASQSIHKNLAWYQQKPGKAPKFLIYYANSLQTGVPSRFSGSGSGTDFTLTISSLQPEDFATYFCQQYYSGWTFGGGTKVEIK

SEQ ID NO: 80 - protein, 106 aa, synthetic construct
DIQMTQSPSSLSASVGDRVTITCKASQSIHKNLAWYQQKPGKAPKLLIYYANSLQTGIPSRFSGSGSGTDFTLTISSLQPEDFATYYCQQYYSGWTFGGGTKVEIK

SEQ ID NO: 81 - protein, 11 aa, synthetic construct
GYSITSDYAWN

SEQ ID NO: 82 - protein, 16 aa, synthetic construct
YISNSGSASYNPSLKS

SEQ ID NO: 83 - protein, 12 aa, synthetic construct
LIYYDYGGAMNF

SEQ ID NO: 84 - protein, 11 aa, synthetic construct
KASQGVSTTVA

SEQ ID NO: 85 - protein, 7 aa, synthetic construct
SASYRYT

SEQ ID NO: 86 - protein, 9 aa, synthetic construct
QHYYSTPWT

SEQ ID NO: 87 - protein, 121 aa, synthetic construct
DVQLQESGPGLVKPSQSLSLTCTVTGYSITSDYAWNWIRQFPGNKLEWMGYISNSGSASYNPSLKSRISITRDTSKNQFFLQLNSVTTEDTATYYCATLIYYDYGGAMNFWGQGTSVTVSS

SEQ ID NO: 88 - protein, 107 aa, synthetic construct
DIVMTQSHKFMSTSVGDRVSITCKASQGVSTTVAWYQQKPGQSPKLLIYSASYRYTGVPDRFTGSGSGTDFTFTISSVQSEDLAVYYCQHYYSTPWTFGGGTKLEIK

SEQ ID NO: 89 - protein, 7 aa, synthetic construct
YASNLQT

SEQ ID NO: 90 - protein, 7 aa, synthetic construct
YASSLQT

SEQ ID NO: 91 - protein, 7 aa, synthetic construct
YASTLQT

SEQ ID NO: 92 - protein, 7 aa, synthetic construct
YATTLQT

SEQ ID NO: 93 - protein, 7 aa, synthetic construct
YASYLQT

SEQ ID NO: 94 - protein, 7 aa, synthetic construct
YANQLQT

SEQ ID NO: 95 - protein, 7 aa, synthetic construct
YAGSLQT

SEQ ID NO: 96 - protein, 7 aa, synthetic construct
YASQLQT

SEQ ID NO: 97 - protein, 7 aa, synthetic construct
YADSLQT

SEQ ID NO: 98 - protein, 8 aa, synthetic construct
QQYYSGFT

SEQ ID NO: 99 - protein, 8 aa, synthetic construct
QQYYSGYT

SEQ ID NO: 100 - protein, 8 aa, synthetic construct
QQYYSGIT

SEQ ID NO: 101 - protein, 8 aa, synthetic construct
QQYYSGVT

SEQ ID NO: 102 - protein, 8 aa, synthetic construct
QQYYSGLT

SEQ ID NO: 103 - protein, 19 aa, synthetic construct
MPSFITLASLSTFEGYFDF

SEQ ID NO: 104 - protein, 19 aa, synthetic construct
MPSFITLASLSTYEGYFDF

SEQ ID NO: 105 - protein, 19 aa, synthetic construct
MPSFITLASLSTIEGYFDF

SEQ ID NO: 106 - protein, 19 aa, synthetic construct
MPSFITLASLSTVEGYFDF

SEQ ID NO: 107 - protein, 19 aa, synthetic construct
MPSFITLASLSTLEGYFDF

SEQ ID NO: 108 - protein, 5 aa, synthetic construct
SYVMH

SEQ ID NO: 109 - protein, 17 aa, synthetic construct
YIDPYNDGAKYNEKFKG

SEQ ID NO: 110 - protein, 10 aa, synthetic construct
GGPYGWYFDV

SEQ ID NO: 111 - protein, 11 aa, synthetic construct
RASEHIYSYLS

SEQ ID NO: 112 - protein, 7 aa, synthetic construct
NAKTLAE

SEQ ID NO: 113 - protein, 9 aa, synthetic construct
QHHFGSPLT

SEQ ID NO: 114 - protein, 119 aa, synthetic construct
EVQLQQSGPELVKPGSSVKMSCKASGYTFSSYVMHWVKQKPGQGLEWIGYIDPYNDGAKYNEKFKGKATLTSDKSSSTAYMELSSLTSEDSAVYYCARGGPYGWYFDVWGAGTTVTVSS

SEQ ID NO: 115 - protein, 107 aa, synthetic construct
DIQMTQSPASLSASVGETVTITCRASEHIYSYLSWYQQKQGKSPQLLVYNAKTLAEGVPSRFSGSGSGTQFSLKINSLQPEDFGTYYCQHHFGSPLTFGAGTTLELK

SEQ ID NO: 116 - protein, 16 aa, synthetic construct
YIDPYNRGAKYNEKFG

SEQ ID NO: 117 - protein, 16 aa, synthetic construct
YIDPYNLGAKYNEKGF

SEQ ID NO: 118 - protein, 16 aa, synthetic construct
YIDPYNKGAKYNEKFG

SEQ ID NO: 119 - protein, 16 aa, synthetic construct
YIDPYNFGAKYNEKFG

SEQ ID NO: 120 - protein, 16 aa, synthetic construct
YIDPYNSGAKYNEKFG

SEQ ID NO: 121 - protein, 16 aa, synthetic construct
YIDPYNYGAKYNEKFG

SEQ ID NO: 122 - protein, 16 aa, synthetic construct
YIDPYNVGAKYNEKFG

SEQ ID NO: 123 - protein, 17 aa, synthetic construct
YIDPYNDRAKYNEKFKG

SEQ ID NO: 124 - protein, 17 aa, synthetic construct
YIDPYNDNAKYNEKFKG

SEQ ID NO: 125 - protein, 17 aa, synthetic construct
YIDPYNDQAKYNEKFKG

SEQ ID NO: 126 - protein, 17 aa, synthetic construct
YIDPYNDEAKYNEKFKG

SEQ ID NO: 127 - protein, 17 aa, synthetic construct
YIDPYNDLAKYNEKFKG

SEQ ID NO: 128 - protein, 17 aa, synthetic construct
YIDPYNDKAKYNEKFKG

SEQ ID NO: 129 - protein, 17 aa, synthetic construct
YIDPYNDSAKYNEKFKG

SEQ ID NO: 130 - protein, 17 aa, synthetic construct
YIDPYNDYAKYNEKFKG

SEQ ID NO: 131 - protein, 17 aa, synthetic construct
YIDPYNDVAKYNEKFKG

SEQ ID NO: 132 - protein, 7 aa, synthetic construct
AAKTLAE

SEQ ID NO: 133 - protein, 7 aa, synthetic construct
YAKTLAE

SEQ ID NO: 134 - protein, 7 aa, synthetic construct
WAKTLAE

SEQ ID NO: 135 - protein, 7 aa, synthetic construct
SAKTLAE

SEQ ID NO: 136 - protein, 7 aa, synthetic construct
TAKTLAE

SEQ ID NO: 137 - protein, 7 aa, synthetic construct
IAKTLAE

SEQ ID NO: 138 - protein, 7 aa, synthetic construct
VAKTLAE

SEQ ID NO: 139 - protein, 7 aa, synthetic construct
NNKTLAE

SEQ ID NO: 140 - protein, 7 aa, synthetic construct
NIKTLAE

SEQ ID NO: 141 - protein, 7 aa, synthetic construct
NLLTLAE

SEQ ID NO: 142 - protein, 7 aa, synthetic construct
NTKTLAE

SEQ ID NO: 143 - protein, 7 aa, synthetic construct
NVKTLAE

SEQ ID NO: 144 - protein, 119 aa, synthetic construct
EVQLVQSGAEVKKPGASVKVSCKASGYTFSSYVMHWVRQAPGQRLEWIGYIDPYNDGAKYSQKFQGRVTLTRDTSASTAYMELSSLRSEDTAVYYCARGGPYGWYFDVWGQGTTVTVSS

SEQ ID NO: 145 - protein, 119 aa, synthetic construct
EVQLVQSGAEVKKPGASVKVSCKASGYTFSSYVMHWVRQAPGQRLEWIGYIDPYNDGAKYSQKFQGRVTLTSDKSASTAYMELSSLRSEDTAVYYCARGGPYGWYFDVWGQGTTVTVSS

SEQ ID NO: 146 - protein, 119 aa, synthetic construct
EVQLVQSGAEVKKPGASVKVSCKASGYTFSSYVMHWVRQAPGQGLEWIGYIDPYNDGAKYAQKFQGRVTLTRDTSTSTVYMELSSLRSEDTAVYYCARGGPYGWYFDVWGQGTTVTVSS

SEQ ID NO: 147 - protein, 119 aa, synthetic construct
EVQLVQSGAEVKKPGASVKVSCKASGYTFSSYVMHWVRQAPGQGLEWIGYIDPYNDGAKYAQKFQGRVTLTSDKSTSTVYMELSSLRSEDTAVYYCARGGPYGWYFDVWGQGTTVTVSS

SEQ ID NO: 148 - protein, 119 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 149 - protein, 119 aa, synthetic construct
EVQLVQSGAEVKKPGASVKVSCKASGYTFSSYVMHWVRQAPGQGLEWIGYIDPYNDGAKYAQKFQGRVTLTSDKSISTAYMELSRLRSDDTVVYYCARGGPYGWYFDVWGQGTTVTVSS

SEQ ID NO: 150 - protein, 107 aa, synthetic construct
DIQMTQSPSSLSASVGDRVTITCRASEHIYSYLSWYQQKPGKAPKLLIYNAKTLAEGVPSRFSGSGSGTDFTLTISSLQPEDFATYYCQHHFGSPLTFGQGTRLEIK

SEQ ID NO: 151 - protein, 107 aa, synthetic construct
DIQMTQSPSSLSASVGDRVTITCRASEHIYSYLSWYQQKPGKAPKLLIYNAKTLAEGVPSRFSGSGSGTQFTLTISSLQPEDFATYYCQHHFGSPLTFGQGTRLEIK

SEQ ID NO: 152 - protein, 107 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 153 - protein, 107 aa, synthetic construct
DIQMTQSPSSLSASVGDRVTITCRASEHIYSYLSWYQQKPGKVPKLLIYNAKTLAEGVPSRFSGSGSGTQFTLTISSLQPEDVATYYCQHHFGSPLTFGQGTRLEIK

SEQ ID NO: 154 - protein, 17 aa, synthetic construct
YIDPYNDGAKYAQKFQG

SEQ ID NO: 155 - protein, 17 aa, synthetic construct
YIDPYNDGAKYSQKFQG

SEQ ID NO: 156 - protein, 116 aa, synthetic construct
QVQLMESGPGLVQPSQTLSLTCTVSGFPLTSYTVHWVRQPPGKGLEWIGAIWSSGSTDYNSALKSRLNINRDSSKSQVFLKMNSLQTEDTAIYFCTKSGWAFFDYWGQGVMVTVSS

SEQ ID NO: 157 - protein, 106 aa, synthetic construct
DIQMTQSPSLLSASVGDRVTLNCIASQNIYKSLAWYQLKLGEAPKLLIYNANSLQAGIPSRFSGSGSGTDFALTISGLQPEDVATYFCQQYSGGYTFGAGTKLELK

SEQ ID NO: 158 - protein, 119 aa, synthetic construct
EVQLVESGGDLVQPGRSLKISCVASGFTFSDYYMAWVRLAPQKGLEWVASISYEGSRTHYGDSVRGRFIISRDNPKNILYLQMNSLGSEDTATYFCARHTGTLDWLVYWGQGTLVIVSS

SEQ ID NO: 159 - protein, 106 aa, synthetic construct
NIVMAQSPKSMSISAGDRVTMNCKASQNVDNNIAWYQQKPGQSPKLLIFYASNRYSGVPDRFTGGGYGTDFTLTIKSVQAEDAAFYYCQRIYNFPTFGSGTKLEIK

SEQ ID NO: 160 - protein, 19 aa, synthetic construct
  VARIANT 13: note = tryptophan, phenylalanine, tyrosine, isoleucine, valine, or leucine
MPSFITLASLSTXEGYFDF

SEQ ID NO: 161 - protein, 7 aa, synthetic construct
  VARIANT 3: note = asparagine, serine, threonine, glycine, or aspartic acid
  VARIANT 4: note = serine, asparagine, serine, threonine, tyrosine, or glutamine
YAXXLQT

SEQ ID NO: 162 - protein, 8 aa, synthetic construct
  VARIANT 7: note = tryptophan, phenylalanine, tyrosine, isoleucine, valine, or leucine
QQYYSGXT

SEQ ID NO: 163 - protein, 127 aa, synthetic construct
  VARIANT 6: note = glutamic acid or glutamine
  VARIANT 9: note = proline or alanine
  VARIANT 12: note = valine or leucine
  VARIANT 15: note = serine or proline
  VARIANT 16: note = glutamine, glutamic acid, or glycine
  VARIANT 17: note = serine or threonine
  VARIANT 23: note = serine, threonine, or alanine
  VARIANT 25: note = threonine or serine
  VARIANT 27: note = serine, glycine, or tyrosine
  VARIANT 30: note = alanine or serine
  VARIANT 37: note = isoleucine or valine
  VARIANT 39: note = lysine or glutamine
  VARIANT 40: note = phenylalanine or proline
  VARIANT 43: note = asparagine or lysine
  VARIANT 44: note = lysine or glycine
  VARIANT 45: note = methionine or leucine
  VARIANT 48: note = methionine or isoleucine
  VARIANT 67: note = isoleucine or valine
  VARIANT 68: note = serine or threonine
  VARIANT 70: note = threonine or serine
  VARIANT 71: note = arginine or valine
  VARIANT 79: note = phenylalanine or serine
  VARIANT 81: note = glutamine or lysine
  VARIANT 83: note = histidine or serine
  VARIANT 87: note = threonine or alanine
  VARIANT 88: note = aspartic acid or alanine
  VARIANT 92: note = threonine or valine
  VARIANT 94: note = serine or tyrosine
  VARIANT 110: note = tryptophan, phenylalanine, tyrosine, isoleucine, valine, or leucine
  VARIANT 119: note = proline or glutamine
  VARIANT 122: note = methionine, valine, leucine, alanine, arginine, asparagine, proline, glutamine, glutamic acid, glycine, isoleucine, histidine, lysine, phenylalanine, serine, threonine, tryptophan, or tyrosine
  VARIANT 123: note = valine, threonine, or leucine
EVQLQXSGXGLXKPXXXLSLTCXVXGXSIXSDYWGWXRXXPGXXXEWXGFITYSGSTSYNPSLKSRXXIXXDTSKNQFXLXLXSVTXXDTAXYXCARMPSFITLASLSTXEGYFDFWGXGTXXTVSS

SEQ ID NO: 164 - protein, 127 aa, synthetic construct
  VARIANT 6: note = glutamic acid or glutamine
  VARIANT 9: note = proline or alanine
  VARIANT 12: note = valine or leucine
  VARIANT 15: note = serine or proline
  VARIANT 16: note = glutamic acid or glycine
  VARIANT 23: note = threonine, alanine, or serine
  VARIANT 25: note = serine or threonine
  VARIANT 27: note = glycine, serine, or tyrosine
  VARIANT 30: note = serine or alanine
  VARIANT 37: note = isoleucine or valine
  VARIANT 39: note = glutamine or lysine
  VARIANT 40: note = proline or phenylalanine
  VARIANT 44: note = glycine or lysine
  VARIANT 45: note = leucine or methionine
  VARIANT 48: note = isoleucine or methionine
  VARIANT 67: note = valine or isoleucine
  VARIANT 71: note = valine or arginine
  VARIANT 83: note = serine or histidine
  VARIANT 88: note = alanine or aspartic acid
  VARIANT 110: note = tryptophan, phenylalanine, tyrosine, isoleucine, valine, or leucine
  VARIANT 122: note = methionine, valine, leucine, alanine, arginine, asparagine, proline, glutamine, glutamic acid, glycine, isoleucine, histidine, lysine, phenylalanine, serine, threonine, tryptophan, or tyrosine
  VARIANT 123: note = valine, threonine, or leucine
EVQLQXSGXGLXKPXXTLSLTCXVXGXSIXSDYWGWXRXXPGKXXEWXGFITYSGSTSYNPSLKSRXTISXDTSKNQFSLKLXSVTAXDTAVYYCARMPSFITLASLSTXEGYFDFWGQGTXXTVSS

SEQ ID NO: 165 - protein, 106 aa, synthetic construct
  VARIANT 10: note = leucine or serine
  VARIANT 21: note = leucine or isoleucine
  VARIANT 22: note = asparagine or threonine
  VARIANT 40: note = leucine or proline
  VARIANT 42: note = glutamic acid or lysine
  VARIANT 43: note = alanine or valine
  VARIANT 46: note = phenylalanine or leucine
  VARIANT 52: note = asparagine, serine, threonine, glycine, or aspartic acid
  VARIANT 53: note = serine, asparagine, threonine, tyrosine, or glutamine
  VARIANT 58: note = isoleucine or valine
  VARIANT 77: note = glycine or serine
  VARIANT 83: note = valine or phenylalanine
  VARIANT 87: note = phenylalanine or tyrosine
  VARIANT 95: note = tryptophan, phenylalanine, tyrosine, isoleucine, valine, or leucine
  VARIANT 105: note = leucine or isoleucine
DIQMTQSPSXLSASVGDRVTXXCKASQSIHKNLAWYQQKXGXXPKXLIYYAXXLQTGXPSRFSGSGSGTDFTLTISXLQPEDXATYXCQQYYSGXTFGGGTKVEXK

SEQ ID NO: 166 - protein, 106 aa, synthetic construct
  VARIANT 43: note = alanine or valine
  VARIANT 46: note = leucine or phenylalanine
  VARIANT 52: note = asparagine, serine, threonine, glycine, or aspartic acid
  VARIANT 53: note = serine, asparagine, threonine, tyrosine, or glutamine
  VARIANT 58: note = valine or isoleucine
  VARIANT 83: note = phenylalanine or valine
  VARIANT 95: note = tryptophan, phenylalanine, tyrosine, isoleucine, valine, or leucine
DIQMTQSPSSLSASVGDRVTITCKASQSIHKNLAWYQQKPGKXPKXLIYYAXXLQTGXPSRFSGSGSGTDFTLTISSLQPEDXATYYCQQYYSGXTFGGGTKVEIK

SEQ ID NO: 167 - protein, 17 aa, synthetic construct
  VARIANT 7: note = aspartic acid, arginine, leucine, lysine, phenylalanine, serine, tyrosine, or valine
  VARIANT 8: note = glycine, arginine, asparagine, glutamine, glutamic acid, leucine, lysine, serine, tyrosine, or valine
  VARIANT 12: note = asparagine, alanine, or serine
  VARIANT 13: note = glutamic acid or glutamine
  VARIANT 16: note = lysine or glutamine
YIDPYNXXAKYXXKFXG

SEQ ID NO: 168 - protein, 7 aa, synthetic construct
  VARIANT 1: note = asparagine, alanine, valine, tryptophan, serine, threonine, arginine, histidine, glycine, isoleucine, or valine
  VARIANT 2: note = alanine, asparagine, isoleucine, leucine, threonine, or valine
XXKTLAE

SEQ ID NO: 169 - protein, 119 aa, synthetic construct
  VARIANT 5: note = glutamine or valine
  VARIANT 9: note = proline or alanine
  VARIANT 11: note = valine or leucine
  VARIANT 12: note = valine or lysine
  VARIANT 16: note = serine or alanine
  VARIANT 20: note = methionine or valine
  VARIANT 38: note = lysine or arginine
  VARIANT 40: note = lysine or alanine
  VARIANT 44: note = glycine or arginine
  VARIANT 56: note = aspartic acid, arginine, leucine, lysine, phenylalanine, serine, tyrosine, or valine
  VARIANT 57: note = glycine, arginine, asparagine, glutamine, glutamic acid, leucine, lysine, serine, tyrosine, or valine
  VARIANT 61: note = asparagine, alanine, or serine
  VARIANT 62: note = glutamic acid or glutamine
  VARIANT 65: note = lysine or glutamine
  VARIANT 67: note = arginine or lysine
  VARIANT 68: note = alanine or valine
  VARIANT 72: note = serine or arginine
  VARIANT 74: note = lysine or threonine
  VARIANT 76: note = serine, isoleucine , alanine, or threonine
  VARIANT 79: note = alanine or valine
  VARIANT 85: note = arginine or serine
  VARIANT 87: note = threonine or arginine
  VARIANT 89: note = aspartic acid or glutamic acid
  VARIANT 91: note = serine or threonine
  VARIANT 92: note = alanine or valine
  VARIANT 104: note = tryptophan, alanine, aspartic acid, glutamic acid, phenylalanine, glycine, isoleucine, lysine, asparagine, glutamine, arginine, serine, threonine, valine, or tyrosine
  VARIANT 111: note = alanine or glutamine
EVQLXQSGXEXXKPGXSVKXSCKASGYTFSSYVMHWVXQXPGQXLEWIGYIDPYNXXAKYXXKFXGXXTLTXDXSXSTXYMELSXLXSXDXXVYYCARGGPYGXYFDVWGXGTTVTVSS

SEQ ID NO: 170 - protein, 119 aa, synthetic construct
  VARIANT 16: note = alanine or serine
  VARIANT 44: note = arginine or glycine
  VARIANT 56: note = aspartic acid, arginine, leucine, lysine, phenylalanine, serine, tyrosine, or valine
  VARIANT 57: note = glycine, arginine, asparagine, glutamine, glutamic acid, leucine, lysine, serine, tyrosine, or valine
  VARIANT 61: note = asparagine, alanine, or serine
  VARIANT 62: note = glutamic acid or glutamine
  VARIANT 65: note = lysine or glutamine
  VARIANT 72: note = arginine or serine
  VARIANT 74: note = threonine or lysine
  VARIANT 76: note = alanine, threonine, or isoleucine
  VARIANT 79: note = alanine or valine
  VARIANT 85: note = serine or arginine
  VARIANT 89: note = glutamic acid or aspartic acid
  VARIANT 92: note = alanine or valine
  VARIANT 104: note = tryptophan, alanine, aspartic acid, glutamic acid, phenylalanine, glycine, isoleucine, lysine, asparagine, glutamine, arginine, serine, threonine, valine, or tyrosine
EVQLVQSGAEVKKPGXSVKVSCKASGYTFSSYVMHWVRQAPGQXLEWIGYIDPYNXXAKYXXKFXGRVTLTXDXSXSTXYMELSXLRSXDTXVYYCARGGPYGXYFDVWGQGTTVTVSS

SEQ ID NO: 171 - protein, 107 aa, synthetic construct
  VARIANT 9: note = alanine or serine
  VARIANT 17: note = glutamic acid or aspartic acid
  VARIANT 18: note = threonine or arginine
  VARIANT 40: note = glutamine or proline
  VARIANT 43: note = serine, alanine, or valine
  VARIANT 45: note = glutamine or lysine
  VARIANT 48: note = valine or isoleucine
  VARIANT 50: note = asparagine, alanine, tyrosine, tryptophan, serine, threonine, isoleucine, or valine
  VARIANT 51: note = alanine, asparagine, isoleucine, leucine, threonine, or valine
  VARIANT 70: note = glutamine or aspartic acid
  VARIANT 72: note = serine or threonine
  VARIANT 74: note = lysine or threonine
  VARIANT 76: note = asparagine or serine
  VARIANT 83: note = phenylalanine or valine
  VARIANT 84: note = glycine or alanine
  VARIANT 100: note = alanine or glutamine
  VARIANT 103: note = threonine or arginine
  VARIANT 106: note = leucine or isoleucine
DIQMTQSPXSLSASVGXXVTITCRASEHIYSYLSWYQQKXGKXPXLLXYXXKTLAEGVPSRFSGSGSGTXFXLXIXSLQPEDXXTYYCQHHFGSPLTFGXGTXLEXK

SEQ ID NO: 172 - protein, 107 aa, synthetic construct
  VARIANT 43: note = alanine or valine
  VARIANT 50: note = asparagine, alanine, tyrosine, tryptophan, serine, threonine, isoleucine, or valine
  VARIANT 51: note = alanine, asparagine, isoleucine, leucine, threonine, or valine
  VARIANT 70: note = aspartic acid or glutamine
  VARIANT 83: note = phenylalanine or valine
DIQMTQSPSSLSASVGDRVTITCRASEHIYSYLSWYQQKPGKXPKLLIYXXKTLAEGVPSRFSGSGSGTXFTLTISSLQPEDXATYYCQHHFGSPLTFGQGTRLEIK

SEQ ID NO: 173 - protein, 10 aa, synthetic construct
  VARIANT 6: note = tryptophan, alanine, aspartic acid, glutamic acid, phenylalanine, glycine, isoleucine, lysine, asparagine, glutamine, arginine, serine, threonine, valine, or tyrosine
GGPYGXYFDV

SEQ ID NO: 174 - protein, 10 aa, synthetic construct
GGPYGAYFDV

SEQ ID NO: 175 - protein, 10 aa, synthetic construct
GGPYGDYFDV

SEQ ID NO: 176 - protein, 10 aa, synthetic construct
GGPYGEYFDV

SEQ ID NO: 177 - protein, 10 aa, synthetic construct
GGPYGFYFDV

SEQ ID NO: 178 - protein, 10 aa, synthetic construct
GGPYGGYFDV

SEQ ID NO: 179 - protein, 10 aa, synthetic construct
GGPYGIYFDV

SEQ ID NO: 180 - protein, 10 aa, synthetic construct
GGPYGKYFDV

SEQ ID NO: 181 - protein, 10 aa, synthetic construct
GGPYGNYFDV

SEQ ID NO: 182 - protein, 10 aa, synthetic construct
GGPYGQYFDV

SEQ ID NO: 183 - protein, 10 aa, synthetic construct
GGPYGRYFDV

SEQ ID NO: 184 - protein, 10 aa, synthetic construct
GGPYGSYFDV

SEQ ID NO: 185 - protein, 10 aa, synthetic construct
GGPYGTYFDV

SEQ ID NO: 186 - protein, 10 aa, synthetic construct
GGPYGVYFDV

SEQ ID NO: 187 - protein, 10 aa, synthetic construct
GGPYGYYFDV

SEQ ID NO: 188 - protein, 5 aa, synthetic construct
GAWMD

SEQ ID NO: 189 - protein, 19 aa, synthetic construct
EIRTKVNNHATNYGESVKG

SEQ ID NO: 190 - protein, 10 aa, synthetic construct
ALYDGFYFDY

SEQ ID NO: 191 - protein, 12 aa, synthetic construct
SASSSVSSGYLY

SEQ ID NO: 192 - protein, 7 aa, synthetic construct
GTSTLAS

SEQ ID NO: 193 - protein, 9 aa, synthetic construct
HQWSSFPYT

SEQ ID NO: 194 - protein, 121 aa, synthetic construct
EVKLEESGGGLVQPGGSMKLSCVASGFTFSGAWMDWVRQSPEKGLEWVAEIRTKVNNHATNYGESVKGRFTISRDDSKSSVYLQMNNLRAEDSGIYYCRGALYDGFYFDYWGQGTTLTVSS

SEQ ID NO: 195 - protein, 108 aa, synthetic construct
QIVLTQSPAIMSASPGEKVNLTCSASSSVSSGYLYWYQQKPGSSPKLWIYGTSTLASGVPARFSGSGSGTSYSLTISNMEAEDAASYFCHQWSSFPYTFGGGTKLEMK

SEQ ID NO: 196 - protein, 121 aa, synthetic construct
  VARIANT 33: note = tryptophan, alanine, arginine, asparagine, aspartic acid, glutamine, glutamic acid, glycine, histidine, isoleucine, leucine, lysine, phenylalanine, proline, serine, threonine, tyrosine, or valine
  VARIANT 34: note = methionine, valine, leucine, isoleucine, glycine, alanine, serine, or threonine
  VARIANT 76: note = aspartic acid, alanine, arginine, asparagine, glutamine, glutamic acid, glycine, histidine, isoleucine, leucine, lysine, phenylalanine, serine, threonine, tyrosine, or valine
  VARIANT 79: note = serine or asparagine
  VARIANT 80: note = threonine or serine
  VARIANT 85: note = methionine or leucine
  VARIANT 86: note = asparagine or serine
  VARIANT 87: note = serine or asparagine
  VARIANT 93: note = threonine or serine
  VARIANT 104: note = aspartic acid, alanine, arginine, asparagine, glutamine, glutamic acid, glycine, histidine, isoleucine, leucine, lysine, phenylalanine, proline, serine, threonine, tryptophan, tyrosine, or valine
  VARIANT 105: note = glycine, alanine, arginine, asparagine, aspartic acid, glutamine, glutamic acid, histidine, isoleucine, leucine, lysine, phenylalanine, proline, serine, threonine, tryptophan, tyrosine, or valine
EVQLVESGGGLVQPGGSLKLSCAASGFTFSGAXXDWVRQAPGKGLEWVAEIRTKVNNHATNYGESVKGRFTISRDXSKXXVYLQXXXLRAEDXAVYYCRGALYXXFYFDYWGQGTLVTVSS

SEQ ID NO: 197 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDGFYFDYWGQGTLVTVSS

SEQ ID NO: 198 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDGFYFDYWGQGTLVTVSS

SEQ ID NO: 199 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDGFYFDYWGQGTLVTVSS

SEQ ID NO: 200 - protein, 108 aa, synthetic construct
  VARIANT 20: note = threonine or serine
  VARIANT 46: note = lysine or arginine
  VARIANT 48: note = tryptophan, alanine, arginine, asparagine, aspartic acid, glutamine, glutamic acid, glycine, histidine, isoleucine, leucine, lysine, phenylalanine, proline, serine, threonine, tyrosine, or valine
  VARIANT 59: note = valine or isoleucine
  VARIANT 79: note = leucine, valine, or isoleucine
  VARIANT 84: note = phenylalanine, valine, leucine, isoleucine, or threonine
  VARIANT 92: note = tryptophan, alanine, arginine, asparagine, aspartic acid, glutamine, glutamic acid, glycine, histidine, isoleucine, leucine, lysine, phenylalanine, proline, serine, threonine, tyrosine, or valine
  VARIANT 107: note = isoleucine or leucine
EIVLTQSPATLSLSPGERAXLSCSASSSVSSGYLYWYQQKPGQAPXLXIYGTSTLASGXPARFSGSGSGTDYTLTISSXEPEDXAVYYCHQXSSFPYTFGQGTKLEXK

SEQ ID NO: 201 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQWSSFPYTFGQGTKLEIK

SEQ ID NO: 202 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQWSSFPYTFGQGTKLEIK

SEQ ID NO: 203 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQWSSFPYTFGQGTKLEIK

SEQ ID NO: 204 - protein, 5 aa, synthetic construct
EFTMH

SEQ ID NO: 205 - protein, 17 aa, synthetic construct
GLKPDNGGISYNQKFKG

SEQ ID NO: 206 - protein, 10 aa, synthetic construct
GAYYRYDADY

SEQ ID NO: 207 - protein, 11 aa, synthetic construct
KASQDVKTAVA

SEQ ID NO: 208 - protein, 7 aa, synthetic construct
SASYRNT

SEQ ID NO: 209 - protein, 9 aa, synthetic construct
QQHYSTPFT

SEQ ID NO: 210 - protein, 119 aa, synthetic construct
EVQLQQSGPELVKPGASVKISCKTSGYTFTEFTMHWVKQSHGKSLEWIGGLKPDNGGISYNQKFKGRATLAVDKSSNTAYMELRSLTSEDSAVYYCARGAYYRYDADYWGQGTTLTVSS

SEQ ID NO: 211 - protein, 107 aa, synthetic construct
DIVLTQSHKFMSTSVGDRVSITCKASQDVKTAVAWYQQKSGQSPKLLIYSASYRNTGVPDRFTGSGSGTDFTFTIDSVQAEDLAVYFCQQHYSTPFTFGTGTKLELK

SEQ ID NO: 212 - protein, 119 aa, synthetic construct
  VARIANT 24: note = threonine or valine
  VARIANT 34: note = methionine, valine, leucine, isoleucine, glycine, alanine, serine, or threonine
  VARIANT 38: note = lysine or arginine
  VARIANT 55: note = asparagine, alanine, arginine, aspartic acid, glutamine, glutamic acid, glycine, histidine, isoleucine, leucine, lysine, phenylalanine, proline, serine, threonine, tryptophan, tyrosine, or valine
  VARIANT 56: note = glycine, alanine, arginine, asparagine, aspartic acid, glutamine, glutamic acid, histidine, isoleucine, leucine, lysine, phenylalanine, proline, serine, threonine, tryptophan, tyrosine, or valine
  VARIANT 74: note = lysine, threonine, aspartic acid, or serine
  VARIANT 77: note = asparagine or serine
  VARIANT 81: note = methionine, valine, leucine, isoleucine, glycine, alanine, serine, or threonine
  VARIANT 91: note = threonine or serine
  VARIANT 105: note = aspartic acid, alanine, arginine, asparagine, glutamine, glutamic acid, glycine, histidine, isoleucine, leucine, lysine, phenylalanine, proline, serine, threonine, tryptophan, tyrosine, or valine
  VARIANT 106: note = alanine, arginine, asparagine, aspartic acid, glutamine, glutamic acid, glycine, histidine, isoleucine, leucine, lysine, phenylalanine, proline, serine, threonine, tryptophan, tyrosine, valine, or methionine
EVQLVQSGAEVKKPGASVKISCKXSGYTFTEFTXHWVXQAPGKGLEWIGGLKPDXXGISYNQKFKGRATLTVDXSTXTAYXELSSLRSEDXAVYYCARGAYYRYXXDYWGQGTLVTVSS

SEQ ID NO: 213 - protein, 119 aa, synthetic construct
EVQLVQSGAEVKKPGASVKISCKTSGYTFTEFTMHWVKQAPGKGLEWIGGLKPDNGGISYNQKFKGRATLTVDKSTNTAYMELSSLRSEDTAVYYCARGAYYRYDADYWGQGTLVTVSS

SEQ ID NO: 214 - protein, 119 aa, synthetic construct
EVQLVQSGAEVKKPGASVKISCKTSGYTFTEFTMHWVRQAPGKGLEWIGGLKPDNGGISYNQKFKGRATLTVDKSTSTAYMELSSLRSEDTAVYYCARGAYYRYDADYWGQGTLVTVSS

SEQ ID NO: 215 - protein, 119 aa, synthetic construct
EVQLVQSGAEVKKPGASVKISCKVSGYTFTEFTMHWVRQAPGKGLEWIGGLKPDNGGISYNQKFKGRATLTVDTSTSTAYMELSSLRSEDTAVYYCARGAYYRYDADYWGQGTLVTVSS

SEQ ID NO: 216 - protein, 107 aa, synthetic construct
  VARIANT 55: note = asparagine, glutamine, aspartic acid, or glutamic acid
  VARIANT 56: note = threonine, serine, or alanine
  VARIANT 60: note = aspartic acid or serine
  VARIANT 73: note = phenylalanine or leucine
DIQLTQSPSSLSASVGDRVTITCKASQDVKTAVAWYQQKPGKAPKLLIYSASYRXXGVPXRFSGSGSGTDFTXTISSLQPEDFATYYCQQHYSTPFTFGQGTKLEIK

SEQ ID NO: 217 - protein, 107 aa, synthetic construct
DIQLTQSPSSLSASVGDRVTITCKASQDVKTAVAWYQQKPGKAPKLLIYSASYRNTGVPDRFSGSGSGTDFTFTISSLQPEDFATYYCQQHYSTPFTFGQGTKLEIK

SEQ ID NO: 218 - protein, 107 aa, synthetic construct
DIQLTQSPSSLSASVGDRVTITCKASQDVKTAVAWYQQKPGKAPKLLIYSASYRNTGVPSRFSGSGSGTDFTFTISSLQPEDFATYYCQQHYSTPFTFGQGTKLEIK

SEQ ID NO: 219 - protein, 107 aa, synthetic construct
DIQLTQSPSSLSASVGDRVTITCKASQDVKTAVAWYQQKPGKAPKLLIYSASYRNTGVPSRFSGSGSGTDFTLTISSLQPEDFATYYCQQHYSTPFTFGQGTKLEIK

SEQ ID NO: 220 - protein, 10 aa, synthetic construct
ALYEGFYFDY

SEQ ID NO: 221 - protein, 10 aa, synthetic construct
ALYDAFYFDY

SEQ ID NO: 222 - protein, 10 aa, synthetic construct
ALYDSFYFDY

SEQ ID NO: 223 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYEGFYFDYWGQGTLVTVSS

SEQ ID NO: 224 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDAFYFDYWGQGTLVTVSS

SEQ ID NO: 225 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDSFYFDYWGQGTLVTVSS

SEQ ID NO: 226 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDEFYFDYWGQGTLVTVSS

SEQ ID NO: 227 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDAFYFDYWGQGTLVTVSS

SEQ ID NO: 228 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDSFYFDYWGQGTLVTVSS

SEQ ID NO: 229 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYEGFYFDYWGQGTLVTVSS

SEQ ID NO: 230 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDAFYFDYWGQGTLVTVSS

SEQ ID NO: 231 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDSFYFDYWGQGTLVTVSS

SEQ ID NO: 232 - protein, 9 aa, synthetic construct
HQASSFPYT

SEQ ID NO: 233 - protein, 9 aa, synthetic construct
HQDSSFPYT

SEQ ID NO: 234 - protein, 9 aa, synthetic construct
HQESSFPYT

SEQ ID NO: 235 - protein, 9 aa, synthetic construct
HQFSSFPYT

SEQ ID NO: 236 - protein, 9 aa, synthetic construct
HQGSSFPYT

SEQ ID NO: 237 - protein, 9 aa, synthetic construct
HQHSSFPYT

SEQ ID NO: 238 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQASSFPYTFGQGTKLEIK

SEQ ID NO: 239 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQDSSFPYTFGQGTKLEIK

SEQ ID NO: 240 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQESSFPYTFGQGTKLEIK

SEQ ID NO: 241 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQFSSFPYTFGQGTKLEIK

SEQ ID NO: 242 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQGSSFPYTFGQGTKLEIK

SEQ ID NO: 243 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQHSSFPYTFGQGTKLEIK

SEQ ID NO: 244 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQASSFPYTFGQGTKLEIK

SEQ ID NO: 245 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQDSSFPYTFGQGTKLEIK

SEQ ID NO: 246 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQESSFPYTFGQGTKLEIK

SEQ ID NO: 247 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQFSSFPYTFGQGTKLEIK

SEQ ID NO: 248 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQGSSFPYTFGQGTKLEIK

SEQ ID NO: 249 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQHSSFPYTFGQGTKLEIK

SEQ ID NO: 250 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQASSFPYTFGQGTKLEIK

SEQ ID NO: 251 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQDSSFPYTFGQGTKLEIK

SEQ ID NO: 252 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQESSFPYTFGQGTKLEIK

SEQ ID NO: 253 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQFSSFPYTFGQGTKLEIK

SEQ ID NO: 254 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQGSSFPYTFGQGTKLEIK

SEQ ID NO: 255 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQHSSFPYTFGQGTKLEIK

SEQ ID NO: 256 - protein, 17 aa, synthetic construct
GLKPDQGGISYNQKFKG

SEQ ID NO: 257 - protein, 17 aa, synthetic construct
GLKPDDGGISYNQKFKG

SEQ ID NO: 258 - protein, 17 aa, synthetic construct
GLKPDNAGISYNQKFKG

SEQ ID NO: 259 - protein, 17 aa, synthetic construct
GLKPDTGGISYNQKFKG

SEQ ID NO: 260 - protein, 17 aa, synthetic construct
GLKPDSGGISYNQKFKG

SEQ ID NO: 261 - protein, 17 aa, synthetic construct
GLKPDGGGISYNQKFKG

SEQ ID NO: 262 - protein, 17 aa, synthetic construct
GLKPDNSGISYNQKFKG

SEQ ID NO: 263 - protein, 17 aa, synthetic construct
GLKPDNTGISYNQKFKG

SEQ ID NO: 264 - protein, 119 aa, synthetic construct
EVQLVQSGAEVKKPGASVKISCKTSGYTFTEFTMHWVKQAPGKGLEWIGGLKPDQGGISYNQKFKGRATLTVDKSTNTAYMELSSLRSEDTAVYYCARGAYYRYDADYWGQGTLVTVSS

SEQ ID NO: 265 - protein, 119 aa, synthetic construct
EVQLVQSGAEVKKPGASVKISCKTSGYTFTEFTMHWVKQAPGKGLEWIGGLKPDDGGISYNQKFKGRATLTVDKSTNTAYMELSSLRSEDTAVYYCARGAYYRYDADYWGQGTLVTVSS

SEQ ID NO: 266 - protein, 119 aa, synthetic construct
EVQLVQSGAEVKKPGASVKISCKTSGYTFTEFTMHWVKQAPGKGLEWIGGLKPDNAGISYNQKFKGRATLTVDKSTNTAYMELSSLRSEDTAVYYCARGAYYRYDADYWGQGTLVTVSS

SEQ ID NO: 267 - protein, 119 aa, synthetic construct
EVQLVQSGAEVKKPGASVKISCKTSGYTFTEFTMHWVKQAPGKGLEWIGGLKPDTGGISYNQKFKGRATLTVDKSTNTAYMELSSLRSEDTAVYYCARGAYYRYDADYWGQGTLVTVSS

SEQ ID NO: 268 - protein, 119 aa, synthetic construct
EVQLVQSGAEVKKPGASVKISCKTSGYTFTEFTMHWVKQAPGKGLEWIGGLKPDSGGISYNQKFKGRATLTVDKSTNTAYMELSSLRSEDTAVYYCARGAYYRYDADYWGQGTLVTVSS

SEQ ID NO: 269 - protein, 119 aa, synthetic construct
EVQLVQSGAEVKKPGASVKISCKTSGYTFTEFTMHWVKQAPGKGLEWIGGLKPDGGGISYNQKFKGRATLTVDKSTNTAYMELSSLRSEDTAVYYCARGAYYRYDADYWGQGTLVTVSS

SEQ ID NO: 270 - protein, 119 aa, synthetic construct
EVQLVQSGAEVKKPGASVKISCKTSGYTFTEFTMHWVKQAPGKGLEWIGGLKPDNSGISYNQKFKGRATLTVDKSTNTAYMELSSLRSEDTAVYYCARGAYYRYDADYWGQGTLVTVSS

SEQ ID NO: 271 - protein, 119 aa, synthetic construct
EVQLVQSGAEVKKPGASVKISCKTSGYTFTEFTMHWVKQAPGKGLEWIGGLKPDNTGISYNQKFKGRATLTVDKSTNTAYMELSSLRSEDTAVYYCARGAYYRYDADYWGQGTLVTVSS

SEQ ID NO: 272 - protein, 119 aa, synthetic construct
EVQLVQSGAEVKKPGASVKISCKTSGYTFTEFTMHWVRQAPGKGLEWIGGLKPDQGGISYNQKFKGRATLTVDKSTSTAYMELSSLRSEDTAVYYCARGAYYRYDADYWGQGTLVTVSS

SEQ ID NO: 273 - protein, 119 aa, synthetic construct
EVQLVQSGAEVKKPGASVKISCKTSGYTFTEFTMHWVRQAPGKGLEWIGGLKPDDGGISYNQKFKGRATLTVDKSTSTAYMELSSLRSEDTAVYYCARGAYYRYDADYWGQGTLVTVSS

SEQ ID NO: 274 - protein, 119 aa, synthetic construct
EVQLVQSGAEVKKPGASVKISCKTSGYTFTEFTMHWVRQAPGKGLEWIGGLKPDNAGISYNQKFKGRATLTVDKSTSTAYMELSSLRSEDTAVYYCARGAYYRYDADYWGQGTLVTVSS

SEQ ID NO: 275 - protein, 119 aa, synthetic construct
EVQLVQSGAEVKKPGASVKISCKTSGYTFTEFTMHWVRQAPGKGLEWIGGLKPDTGGISYNQKFKGRATLTVDKSTSTAYMELSSLRSEDTAVYYCARGAYYRYDADYWGQGTLVTVSS

SEQ ID NO: 276 - protein, 119 aa, synthetic construct
EVQLVQSGAEVKKPGASVKISCKTSGYTFTEFTMHWVRQAPGKGLEWIGGLKPDSGGISYNQKFKGRATLTVDKSTSTAYMELSSLRSEDTAVYYCARGAYYRYDADYWGQGTLVTVSS

SEQ ID NO: 277 - protein, 119 aa, synthetic construct
EVQLVQSGAEVKKPGASVKISCKTSGYTFTEFTMHWVRQAPGKGLEWIGGLKPDGGGISYNQKFKGRATLTVDKSTSTAYMELSSLRSEDTAVYYCARGAYYRYDADYWGQGTLVTVSS

SEQ ID NO: 278 - protein, 119 aa, synthetic construct
EVQLVQSGAEVKKPGASVKISCKTSGYTFTEFTMHWVRQAPGKGLEWIGGLKPDNSGISYNQKFKGRATLTVDKSTSTAYMELSSLRSEDTAVYYCARGAYYRYDADYWGQGTLVTVSS

SEQ ID NO: 279 - protein, 119 aa, synthetic construct
EVQLVQSGAEVKKPGASVKISCKTSGYTFTEFTMHWVRQAPGKGLEWIGGLKPDNTGISYNQKFKGRATLTVDKSTSTAYMELSSLRSEDTAVYYCARGAYYRYDADYWGQGTLVTVSS

SEQ ID NO: 280 - protein, 119 aa, synthetic construct
EVQLVQSGAEVKKPGASVKISCKVSGYTFTEFTMHWVRQAPGKGLEWIGGLKPDQGGISYNQKFKGRATLTVDTSTSTAYMELSSLRSEDTAVYYCARGAYYRYDADYWGQGTLVTVSS

SEQ ID NO: 281 - protein, 119 aa, synthetic construct
EVQLVQSGAEVKKPGASVKISCKVSGYTFTEFTMHWVRQAPGKGLEWIGGLKPDDGGISYNQKFKGRATLTVDTSTSTAYMELSSLRSEDTAVYYCARGAYYRYDADYWGQGTLVTVSS

SEQ ID NO: 282 - protein, 119 aa, synthetic construct
EVQLVQSGAEVKKPGASVKISCKVSGYTFTEFTMHWVRQAPGKGLEWIGGLKPDNAGISYNQKFKGRATLTVDTSTSTAYMELSSLRSEDTAVYYCARGAYYRYDADYWGQGTLVTVSS

SEQ ID NO: 283 - protein, 119 aa, synthetic construct
EVQLVQSGAEVKKPGASVKISCKVSGYTFTEFTMHWVRQAPGKGLEWIGGLKPDTGGISYNQKFKGRATLTVDTSTSTAYMELSSLRSEDTAVYYCARGAYYRYDADYWGQGTLVTVSS

SEQ ID NO: 284 - protein, 119 aa, synthetic construct
EVQLVQSGAEVKKPGASVKISCKVSGYTFTEFTMHWVRQAPGKGLEWIGGLKPDSGGISYNQKFKGRATLTVDTSTSTAYMELSSLRSEDTAVYYCARGAYYRYDADYWGQGTLVTVSS

SEQ ID NO: 285 - protein, 119 aa, synthetic construct
EVQLVQSGAEVKKPGASVKISCKVSGYTFTEFTMHWVRQAPGKGLEWIGGLKPDGGGISYNQKFKGRATLTVDTSTSTAYMELSSLRSEDTAVYYCARGAYYRYDADYWGQGTLVTVSS

SEQ ID NO: 286 - protein, 119 aa, synthetic construct
EVQLVQSGAEVKKPGASVKISCKVSGYTFTEFTMHWVRQAPGKGLEWIGGLKPDNSGISYNQKFKGRATLTVDTSTSTAYMELSSLRSEDTAVYYCARGAYYRYDADYWGQGTLVTVSS

SEQ ID NO: 287 - protein, 119 aa, synthetic construct
EVQLVQSGAEVKKPGASVKISCKVSGYTFTEFTMHWVRQAPGKGLEWIGGLKPDNTGISYNQKFKGRATLTVDTSTSTAYMELSSLRSEDTAVYYCARGAYYRYDADYWGQGTLVTVSS

SEQ ID NO: 288 - protein, 15 aa, Homo sapiens
SSTTAQVNWEQQDQL

SEQ ID NO: 289 - protein, 9 aa, Homo sapiens
IYHTYPDGT

SEQ ID NO: 290 - protein, 5 aa, Homo sapiens
GRIFL

SEQ ID NO: 291 - protein, 330 aa, synthetic construct [sequence omitted]

SEQ ID NO: 292 - protein, 325 aa, synthetic construct
TKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 293 - protein, 105 aa, synthetic construct [sequence omitted]

SEQ ID NO: 294 - protein, 214 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 449 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 296 - protein, 438 aa, synthetic construct
QVQLQQPGAELVRPGSSVKLSCKASGYTFNHYWIHWVKQGPLQGLEWIGSIDPSDSEVNYNHKFRDKATLTVDKSSSTAYMHLSSLTSEDSAVYYCAKGLRDSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 297 - protein, 213 aa, synthetic construct
QIVLTQSPAIMSASLGEEVTLTCSATSSVNFMHWYQQKSGTSPKLLIYSTSILASGVPSRFSGSGSGTFYSLTISSVEAEDAADYYCHQWTSYPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 298 - protein, 444 aa, synthetic construct
QVQLQQPGAELVRPGSSVKLSCKASGYTFNHYWIHWVKQGPLQGLEWIGSIDPSDSEVNYNHKFRDKATLTVDKSSSTAYMHLSSLTSEDSAVYYCAKGLRDSWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 299 - protein, 213 aa, synthetic construct; identical to SEQ ID NO: 297

SEQ ID NO: 300 - protein, 443 aa, synthetic construct
EVQLVESGGGLVQPGGSLKLSCAASGFTFSNSGLAWVRQAPEKGLEWVATITYNGTSTYYRDSVKGRFTISRDNAKNTLYLQMSSLRSEDTATYYCARWVPGSGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 301 - protein, 218 aa, synthetic construct
DIVLTQSPASLAVSLGQRATISCRASQSVTISRYTLMHWYQQKPGQPPKLLIYRASNLASGIPARFSGSGSGTDFTLNIHPVEEDDAATYYCQQSRESPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 302 - protein, 446 aa, synthetic construct
QVQLMESGPGLVQPSQTLSLTCTVSGFPLTSYTVHWVRQPPGKGLEWIGAIWSSGSTDYNSALKSRLNINRDSSKSQVFLKMNSLQTEDTAIYFCTKSGWAFFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 303 - protein, 213 aa, synthetic construct
DIQMTQSPSLLSASVGDRVTLNCIASQNIYKSLAWYQLKLGEAPKLLIYNANSLQAGIPSRFSGSGSGTDFALTISGLQPEDVATYFCQQYSGGYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

(19) United States
(12) Patent Application Publication
(10) Pub. No.: US 2025/0064794 A1
(43) Pub. Date: Feb. 27, 2025

(54) A THERAPEUTIC COMBINATION COMPRISING A TIGIT ANTAGONIST, A PD-1 ANTAGONIST, AND LENVATINIB

(71) Applicants: EISAI R&D MANAGEMENT CO., LTD., TOKYO (JP); Merck Sharp & Dohme LLC, Rahway, NJ (US)

(72) Inventors: Jane Anne Healy, Rahway, NJ (US); Robert John Orlowski, Rahway, NJ (US); Elaine M. Pinheiro, Boston, MA (US); Alexandra Snyder, New York, NY (US); Douglas E. Linn, Boston, MA (US); Sujata Shrawankumar Jha, Rahway, NJ (US)

(21) Appl. No.: 18/682,546

(22) PCT Filed: Aug. 9, 2022
(86) PCT No.: PCT/US22/39765

§ 371 Date: Feb. 9, 2024

Related U.S. Application Data
(60) Provisional application No. 63/326,335, filed on Apr. 1, 2022.
(60) Provisional application No. 63/231,532, filed on Aug. 10, 2021.

(51) Int. Cl. A61K 31/47; A61K 39/395; A61P 35/00

(52) U.S. Cl. CPC A61K 31/47; A61K 39/3955; A61P 35/00

(57) ABSTRACT

Provided herein are methods of treating cancer, an infectious disease, or an infection, which comprise administering to a human patient in need thereof: (a) a TIGIT antagonist; (b) a PD-1 antagonist; and (c) lenvatinib represented by Formula (I), or a pharmaceutically acceptable salt thereof. Also provided are kits containing such agents and uses of therapeutic combinations of such agents for the treatment of cancer, an infectious disease, or an infection.

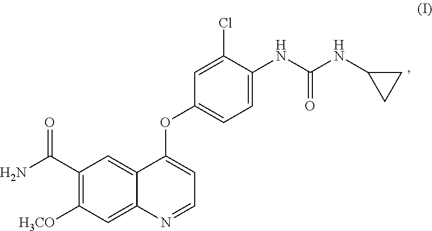 [SMILES: COc1cc2nccc(Oc3ccc(NC(=O)NC4CC4)c(Cl)c3)c2cc1C(N)=O]

### FIELD

Provided herein are methods for treating cancer, an infectious disease, or an infection using a combination of (a) a T cell immunoreceptor with Ig and ITIM domains (TIGIT) antagonist, (b) a programmed death 1 protein (PD-1) antagonist, and (c) 4-[3-chloro-4-(cyclopropylaminocarbonyl)aminophenoxy]-7-methoxy-6-quinolinecarboxamide (lenvatinib) represented by Formula (I),

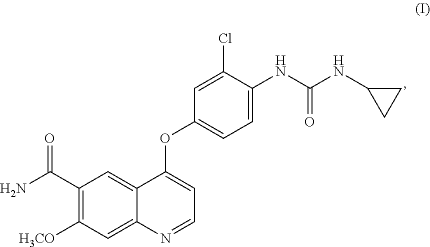 [SMILES: COc1cc2nccc(Oc3ccc(NC(=O)NC4CC4)c(Cl)c3)c2cc1C(N)=O]

or a pharmaceutically acceptable salt thereof.

### CROSS-REFERENCE TO RELATED APPLICATIONS

This application claims the benefit of U.S. Provisional Patent Application No. 63/231,532, filed Aug. 10, 2021 and U.S. Provisional Patent Application No. 63/326,335, filed Apr. 1, 2022, each of which is incorporated by reference herein in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Jul. 25, 2022, is named 25304-WO-PCT_SL.XML and is 352 KB in size.

### BACKGROUND OF THE INVENTION

TIGIT is an immunomodulatory receptor expressed primarily on activated T cells and NK cells. TIGIT is also known as VSIG9, VSTM3, and WUCAM. Its structure shows one extracellular immunoglobulin domain, a type 1 transmembrane region and two ITIM motifs. TIGIT forms part of a co-stimulatory network that consists of positive (CD226) and negative (TIGIT) immunomodulatory receptors on T cells, and ligands expressed on APCs (CD155 and CD112).

An important feature in the structure of TIGIT is the presence of an immunoreceptor tyrosine-based inhibition motif (ITIM) in its cytoplasmic tail domain. As with PD-1, the ITIM domain in the cytoplasmic region of TIGIT is predicted to recruit tyrosine phosphatases, such as SHP-1 and SHP-2, and subsequent de-phosphorylation of tyrosine residues within the immunoreceptor tyrosine-base activation motifs (ITAM) on T cell receptor (TCR) subunits. Hence, ligation of TIGIT by receptor-ligands CD155 and CD112 expressed by tumor cells or TAMS may contribute to the suppression of TCR-signaling and T cell activation, which is essential for mounting effective anti-tumor immunity. Thus, an antagonist antibody specific for TIGIT could inhibit the CD155 and CD112 induced suppression of T cell responses and enhance anti-tumor immunity.

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144 (2013); Robert et al., Lancet, 384:1109-1117 (2014); Robert et al., N Engl J Med, 372: 2521-2532 (2015); Robert et al., N Engl J Med, 372:320-330 (2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, NJ; OPDIVO® (nivolumab), Bristol-Myers Squibb Company, Princeton, NJ) and those that bind to the PD-L1 ligand (e.g., TECENTRIQ® (atezolizumab), Genentech, San Francisco, CA).

Tyrosine kinases are involved in the modulation of growth factor signaling and thus are an important target for cancer therapies. Lenvatinib is a multiple receptor tyrosine kinase (RTK) (multi-RTK) inhibitor that selectively inhibits the kinase activities of vascular endothelial growth factor (VEGF) receptors (VEGFR1 (FLT1), VEGFR2 (KDR) and VEGFR3 (FLT4)), and fibroblast growth factor (FGF) receptors FGFR1, 2, 3 and 4 in addition to other proangiogenic and oncogenic pathway-related RTKs (including the platelet-derived growth factor (PDGF) receptor PDGFRα; KIT; and the RET proto-oncogene (RET)) involved in tumor proliferation. In particular, lenvatinib possesses a new binding mode (Type V) to VEGFR2, as confirmed through X-ray crystal structural analysis, and exhibits rapid and potent inhibition of kinase activity, according to kinetic analysis.

### SUMMARY

The present disclosure provides methods, pharmaceutical compositions, uses and kits of treating a cancer, an infectious disease, or an infection using a combination of therapeutic agents, e.g., a combination of antibodies or antigen binding fragments thereof.

The present disclosure provides methods of treating a cancer, an infectious disease, or an infection using a combination of a TIGIT antagonist (e.g., antibody (e.g., monoclonal antibody) or antigen binding fragment thereof), a PD-1 antagonist (e.g., antibody (e.g., monoclonal antibody) or antigen binding fragment thereof), and a multiple RTK inhibitor (e.g., antibody (e.g., monoclonal antibody) or antigen binding fragment thereof). In specific embodiments the multiple RTK inhibitor antagonizes the VEGFR1, VEGFR2 and VEGFR3 kinases).

Among other things, the present disclosure encompasses insights that certain combinations of immune checkpoint inhibitors (e.g., a TIGIT antagonist and a PD-1 antagonist) in combination with cancerogenic signaling pathway inhibitors (e.g., lenvatinib) as provided herein may enhance efficacy without significant added toxicity as compared with existing treatments. While it has been proposed that the efficacy of anti-TIGIT antagonistic antibodies and anti-PD-1 antagonistic antibodies might be enhanced if administered in combination with other approved or experimental cancer therapies, there are no clear guidelines as to which agent combined with the anti-TIGIT antagonistic antibodies and anti-PD-1 antagonistic antibodies may be effective or in which patients the combination may enhance the efficacy of treatment.

The present disclosure further recognizes that a combination of a TIGIT antagonist, a PD-1 antagonist and lenvatinib provided herein may be beneficial because they have divergent metabolic pathways. For example, there may be no drug interactions from a combination of an TIGIT antagonist, a PD-1 antagonist and lenvatinib. Without wishing to be bound by any particular theory, a TIGIT antagonist and a PD-1 antagonist may be primarily catabolized to small peptides and single amino acids via general protein degradation routes (e.g., without metabolism for clearance), while lenvatinib is metabolized by enzymatic and non-enzymatic processes.

The present disclosure provides methods of treating cancer (e.g., endometrial cancer, hepatocellular cancer, etc.) using a combination of a TIGIT antagonist, a PD-1 antagonist, and 4-[3-chloro-4-(cyclopropylaminocarbonyl)aminophenoxy]-7-methoxy-6-quinolinecarboxamide (lenvatinib) represented by Formula (I),

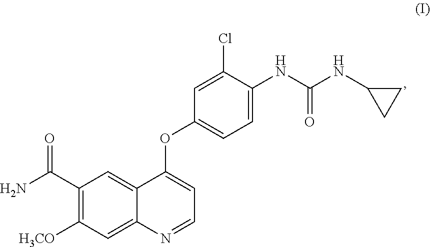 [SMILES: COc1cc2nccc(Oc3ccc(NC(=O)NC4CC4)c(Cl)c3)c2cc1C(N)=O]

or a pharmaceutically acceptable salt thereof.

The present disclosure further provides kits including a TIGIT antagonist, a PD-1 antagonist, and 4-[3-chloro-4-(cyclopropylaminocarbonyl)aminophenoxy]-7-methoxy-6-quinolinecarboxamide (lenvatinib), or a pharmaceutically acceptable salt thereof.

Also provided herein are uses of a therapeutic combination for treating cancer (e.g., endometrial cancer, hepatocellular carcinoma (HCC)), wherein the therapeutic combination includes a TIGIT antagonist, a PD-1 antagonist, and 4-[3-chloro-4-(cyclopropylaminocarbonyl)aminophenoxy]-7-methoxy-6-quinolinecarboxamide (lenvatinib), or a pharmaceutically acceptable salt thereof.

In one aspect, provided herein is a method of treating cancer, comprising administering to a human patient in need thereof:
(a) a TIGIT antagonist;
(b) a PD-1 antagonist; and
(c) 4-[3-chloro-4-(cyclopropylaminocarbonyl)aminophenoxy]-7-methoxy-6-quinolinecarboxamide (lenvatinib), or a pharmaceutically acceptable salt thereof.

In one aspect, provided herein are therapeutic combinations for use in treating cancer, comprising administering to a human patient in need thereof:
(a) a TIGIT antagonist;
(b) a PD-1 antagonist; and
(c) 4-[3-chloro-4-(cyclopropylaminocarbonyl)aminophenoxy]-7-methoxy-6-quinolinecarboxamide (lenvatinib), or a pharmaceutically acceptable salt thereof.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelofibrosis, soft tissue sarcoma, thyroid cancer, endometrial cancer, and carcinoid cancer. In some embodiments, the cancer is selected from the group consisting of endometrial cancer, hepatocellular carcinoma (HCC), cervical cancer, head and neck cancer (HNSCC), biliary cancer, esophageal cancer, and triple negative breast cancer (TNBC). In certain embodiments, the cancer is endometrial cancer or hepatocellular carcinoma. In some embodiments, the cancer is locally recurrent unresectable HCC. In some embodiments, the cancer is metastatic HCC. In some embodiments, the cancer is mismatch repair proficient endometrial cancer.

In certain embodiments, the cancer is metastatic. In some embodiments, the cancer is relapsed. In other embodiments, the cancer is refractory. In yet other embodiments, the cancer is relapsed and refractory. In certain embodiments, the cancer is resectable.

In another aspect, provided herein is a kit comprising:
(a) a TIGIT antagonist;
(b) a PD-1 antagonist; and
(c) 4-[3-chloro-4-(cyclopropylaminocarbonyl)aminophenoxy]-7-methoxy-6-quinolinecarboxamide (lenvatinib) or a pharmaceutically acceptable salt thereof.

In certain embodiments, the kit further comprises instructions for administering to a human patient the TIGIT antagonist, the PD-1 antagonist, and 4-[3-chloro-4-(cyclopropylaminocarbonyl)aminophenoxy]-7-methoxy-6-quinolinecarboxamide (lenvatinib), or a pharmaceutically acceptable salt thereof.

In still another aspect, provided herein is use of a therapeutic combination for treating cancer in a human patient, wherein the therapeutic combination comprises:
(a) a TIGIT antagonist;
(b) a PD-1 antagonist; and
(c) 4-[3-chloro-4-(cyclopropylaminocarbonyl)aminophenoxy]-7-methoxy-6-quinolinecarboxamide (lenvatinib), or a pharmaceutically acceptable salt thereof.

In some embodiments, the cancer is selected from the group consisting of endometrial cancer, hepatocellular carcinoma (HCC), cervical cancer, head and neck cancer (HNSCC), biliary cancer, esophageal cancer, and triple negative breast cancer (TNBC). In some embodiments, the cancer is endometrial cancer. In some embodiments, the cancer is HCC. In some embodiments, the cancer is locally recurrent unresectable HCC. In some embodiments, the cancer is metastatic HCC. In some embodiments, the cancer is mismatch repair proficient endometrial cancer.

In certain embodiments of the methods, pharmaceutical compositions, kits, uses, or the combinations for use provided herein, the subject is a human patient.

In certain embodiments the methods, pharmaceutical compositions, kits, uses, or the combinations for use provided herein are for treating cancer.

In certain embodiments of the methods, pharmaceutical compositions, kits, or uses provided herein, the PD-1 antagonist is an anti-human PD-1 monoclonal antibody or antigen binding fragment thereof.

In other embodiments of the methods, pharmaceutical compositions, kits, or uses provided herein, the PD-1 antagonist is an anti-human PD-L1 monoclonal antibody or antigen binding fragment thereof.

In some embodiments of the methods, pharmaceutical compositions, kits, or uses provided herein, the anti-human PD-1 monoclonal antibody is a humanized antibody.

In other embodiments of the methods, pharmaceutical compositions, kits, or uses provided herein, the anti-human PD-1 monoclonal antibody is a human antibody.

In some embodiments of the methods, pharmaceutical compositions, kits, or uses provided herein, the anti-human PD-L1 monoclonal antibody is a humanized antibody.

In other embodiments of the methods, pharmaceutical compositions, kits, or uses provided herein, the anti-human PD-L1 monoclonal antibody is a human antibody.

In certain embodiments of the methods, pharmaceutical compositions, kits, or uses provided herein, the TIGIT antagonist is an anti-human TIGIT monoclonal antibody or antigen binding fragment thereof.

In some embodiments of the methods, pharmaceutical compositions, kits, or uses provided herein, the anti-human TIGIT monoclonal antibody is a humanized antibody.

In other embodiments of the methods, pharmaceutical compositions, kits, or uses provided herein, the anti-human TIGIT monoclonal antibody is a human antibody.

In still other embodiments of the methods, pharmaceutical compositions, kits, uses provided herein, the anti-PD-1 antibody is independently selected from pembrolizumab, nivolumab, cemiplimab, sintilimab, tislelizumab, camrelizumab and toripalimab.

In one embodiment of the methods, pharmaceutical compositions, kits, or uses provided herein, the anti-human PD-1 monoclonal antibody is pembrolizumab.

In another embodiment of the methods, pharmaceutical compositions, kits, or uses provided herein, the anti-human PD-1 monoclonal antibody is nivolumab.

In another embodiment of the methods, pharmaceutical compositions, kits, or uses provided herein, the anti-human PD-1 monoclonal antibody is cemiplimab.

In yet another embodiment of the methods, pharmaceutical compositions, kits, or uses provided herein, the anti-human PD-1 monoclonal antibody or antigen binding fragment thereof is pidilizumab (U.S. Pat. No. 7,332,582).

In yet another embodiment of the methods, pharmaceutical compositions, kits, or uses provided herein, the anti-human PD-1 monoclonal antibody or antigen binding fragment thereof is AMP-514 (MedImmune LLC, Gaithersburg, MD).

In yet another embodiment of the methods, pharmaceutical compositions, kits, or uses provided herein, the anti-human PD-1 monoclonal antibody or antigen binding fragment thereof is PDR001 (U.S. Pat. No. 9,683,048).

In yet another embodiment of the methods, pharmaceutical compositions, kits, or uses provided herein, the anti-human PD-1 monoclonal antibody or antigen binding fragment thereof is BGB-A317 (U.S. Pat. No. 8,735,553).

In yet another embodiment of the methods, pharmaceutical compositions, kits, or uses provided herein, the anti-human PD-1 monoclonal antibody or antigen binding fragment thereof is MGA012 (MacroGenics, Rockville, MD).

In certain embodiments of the methods, kits, or uses provided herein, the anti-human TIGIT monoclonal antibody comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of the methods, kits, or uses provided herein the anti-human TIGIT monoclonal antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In other embodiments of the methods, kits, or uses provided herein, the anti-human TIGIT monoclonal antibody or antigen binding fragment thereof comprises a light chain comprising or consisting of the amino acid sequence as set forth in SEQ ID NO: 294 and a heavy chain comprising or consisting of the amino acid sequence as set forth in SEQ ID NO: 295.

In yet still another embodiment of the methods, kits, or uses provided herein, the lenvatinib or a pharmaceutically acceptable salt thereof is lenvatinib mesylate. Capsules for oral administration contain 4 mg, 8 mg, 10 mg, 12 mg, 14 mg or 20 mg of lenvatinib.

In one specific embodiment of the methods, kits, or uses provided herein, the PD-1 antagonist is pembrolizumab; and the TIGIT antagonist is a monoclonal antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In one specific embodiment of the methods, kits, or uses provided herein, the PD-1 antagonist is nivolumab; and the TIGIT antagonist is a monoclonal antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In one specific embodiment of the methods, kits, or uses provided herein, the PD-1 antagonist is cemiplimab; and the TIGIT antagonist is a monoclonal antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In another aspect, provided herein is a method of enhancing T cell activity, comprising contacting the T cells with:
(a) an anti-human TIGIT antibody (e.g., monoclonal antibody) or antigen binding fragment thereof,
(b) an anti-human PD-1 antibody (e.g., monoclonal antibody) or antigen binding fragment thereof, and
(c) 4-[3-chloro-4-(cyclopropylaminocarbonyl)aminophenoxy]-7-methoxy-6-quinolinecarboxamide (lenvatinib), or a pharmaceutically acceptable salt thereof.

In some embodiments, the enhancement of T cell activity occurs in vitro. In other embodiments, the enhancement of T cell activity occurs in vivo. For example, the enhancement is in a subject including but not limited to a human subject or human patient.

In certain embodiments, the enhancement of T cell activity is measured by increased cytokine production. In other embodiments, the enhancement of T cell activity is measured by increased cell proliferation.

In some embodiments, provided herein is a method of increasing cytokine production of T cells, comprising contacting the T cells with:
(a) an anti-human TIGIT antibody (e.g., monoclonal antibody) or antigen binding fragment thereof;
(b) an anti-human PD-1 antibody (e.g., monoclonal antibody) or antigen binding fragment thereof; and
(c) 4-[3-chloro-4-(cyclopropylaminocarbonyl)aminophenoxy]-7-methoxy-6-quinolinecarboxamide (lenvatinib), or a pharmaceutically acceptable salt thereof.

In some embodiments, the increased cytokine production of T cells occurs in vitro. In other embodiments, the increased cytokine production of T cells occurs in vivo.

In some embodiments of the methods, kits, or uses described herein, the human patient is administered about 200 mg, about 240 mg, or about 2 mg/kg pembrolizumab, and pembrolizumab is administered once every three weeks. In one embodiment, the human patient is administered about 200 mg pembrolizumab once every three weeks. In one embodiment, the human patient is administered about 240 mg pembrolizumab once every three weeks. In one embodiment, the human patient is administered about 2 mg/kg pembrolizumab once every three weeks.

In certain embodiments of the methods, kits, or uses described herein, the human patient is administered about 400 mg pembrolizumab, and pembrolizumab is administered once every six weeks.

In other embodiments of the methods, kits, or uses described herein, the human patient is administered about 240 mg or about 3 mg/kg nivolumab once every two weeks, or about 480 mg nivolumab once every four weeks. In one specific embodiment, the human patient is administered about 240 mg nivolumab once every two weeks. In one specific embodiment, the human patient is administered about 3 mg/kg nivolumab once every two weeks. In one specific embodiment, the human patient is administered about 480 mg nivolumab once every four weeks.

In yet other embodiments of the methods, kits, or uses described herein, the human patient is administered about 350 mg cemiplimab, and cemiplimab is administered once every three weeks.

In some embodiments of the methods, kits, or uses described herein, the human patient is administered about 200 mg, about 240 mg, or about 2 mg/kg of the anti-human TIGIT monoclonal antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295, and the anti-human TIGIT monoclonal antibody or antigen binding fragment thereof is administered once every three weeks. In one embodiment, the human patient is administered about 200 mg of the anti-human TIGIT monoclonal antibody or antigen binding fragment thereof once every three weeks. In one embodiment, the human patient is administered about 240 mg of the anti-human TIGIT monoclonal antibody or antigen binding fragment thereof once every three weeks. In one embodiment, the human patient is administered about 2 mg/kg of the anti-human TIGIT monoclonal antibody or antigen binding fragment thereof once every three weeks.

In certain embodiments of the methods described herein, the human patient is administered about 400 mg of the anti-human TIGIT monoclonal antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295, and e anti-human TIGIT monoclonal antibody or antigen binding fragment thereof is administered once every six weeks.

In yet still other embodiments of the methods described herein, the human patient is administered about 4, about 8, about 10, about 12, about 14, about 18, about 20, or about 24 mg lenvatinib once daily.

Thus, in some embodiments, the human patient is administered:
(a) about 200 mg, about 240 mg, or about 2 mg/kg pembrolizumab once every three or six weeks;
(b) about 200 mg, about 240 mg, or about 2 mg/kg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 4, about 8, about 10, about 12, about 14, about 18, about 20, or about 24 mg lenvatinib once daily.

In certain embodiments, the human patient is administered:
(a) about 200 mg, about 240 mg, or about 2 mg/kg pembrolizumab once every three weeks;
(b) about 200 mg, about 240 mg, or about 2 mg/kg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks;
(c) about 4, about 8, about 10, about 12, about 14, about 18, about 20, or about 24 mg lenvatinib once daily.

In certain embodiments, the human patient is administered:
(a) about 200 mg pembrolizumab once every three weeks;
(b) about 200 mg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 4, about 8, about 10, about 12, about 14, about 18, about 20, or about 24 mg lenvatinib once daily.

In certain embodiments, the human patient is administered:
(a) about 240 mg pembrolizumab once every three weeks;
(b) about 240 mg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 4, about 8, about 10, about 12, about 14, about 18, about 20, or about 24 mg lenvatinib once daily.

In certain embodiments, the human patient is administered:
(a) about 2 mg/kg pembrolizumab once every three weeks;
(b) about 2 mg/kg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 4, about 8, about 10, about 12, about 14, about 18, about 20, or about 24 mg lenvatinib once daily.

In certain embodiments, the human patient is administered:
(a) about 400 mg pembrolizumab once every six weeks;
(b) about 400 mg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every six weeks; and
(c) about 4, about 8, about 10, about 12, about 14, about 18, about 20, or about 24 mg lenvatinib once daily.

In a specific embodiment, provided herein is a method of treating endometrial cancer (e.g., mismatch repair proficient), comprising administering to a human patient in need thereof:
(a) about 200 mg pembrolizumab once every three weeks;
(b) about 200 mg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 12, about 14, or about 20 mg lenvatinib once daily.

In a specific embodiment, provided herein is a method of treating HCC (e.g., locally recurrent unresectable or metastatic), comprising administering to a human patient in need thereof:
(a) about 200 mg pembrolizumab once every three weeks;
(b) about 200 mg of an anti-human TIGIT antibody or antigen binding fragment thereof comprising three light chains CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 4, about 8 or about 12 mg lenvatinib once daily.

In certain embodiments of the methods, kits and uses provided herein, the anti-human TIGIT monoclonal antibody and the anti-human PD-1 monoclonal antibody are administered on the same day. In some embodiments, the anti-human TIGIT monoclonal antibody and the anti-human PD-1 monoclonal antibody are administered sequentially. In some embodiments, the anti-human TIGIT monoclonal antibody and the anti-human PD-1 monoclonal antibody are administered concurrently. In some embodiments, the anti-human TIGIT monoclonal antibody and the anti-human PD-1 monoclonal antibody are co-formulated.

In some embodiments of the methods, kits, or uses described herein, a pharmaceutically acceptable salt of lenvatinib (e.g., lenvatinib mesylate) can be used. When a pharmaceutically acceptable salt of lenvatinib mesylate is used, the dosage of the pharmaceutically acceptable salt of lenvatinib mesylate is appropriately adjusted to provide the same molar equivalents of lenvatinib as about 4, about 8, about 10, about 12, about 14, about 18, about 20, or about 24 mg lenvatinib provides.

### BRIEF DESCRIPTION OF THE FIGURES

FIG. 1 shows average tumor volumes in each treatment group for one or more of cancer treatments, including the combination treatment of a PD-1 antagonist, a TIGIT antagonist, and lenvatinib.

FIG. 2 shows individual mouse tumor growth curves for one or more of cancer treatments, including the combination treatment of a PD-1 antagonist, a TIGIT antagonist, and lenvatinib.

FIG. 3 shows changes in tumor in mouse body weights during course of specified treatments.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-TIGIT antibody or antigen binding fragment thereof, an anti-PD-1 antibody or antigen binding fragment thereof, or lenvatinib, or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-TIGIT antibody, an anti-PD-1 antibody, and lenvatinib as described herein) into a patient, such as by oral, mucosal, intradermal, intravenous, subcutaneous, intramuscular delivery, and/or any other methods of physical delivery described herein or known in the art.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, fusion proteins comprising an antigen binding fragment thereof that competes with the intact antibody for specific.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.”

Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. As referred to herein the light chain CDRs are CDRL1, CDRL2 and CDRL3, respectively, and the heavy chain CDRs are CDRH1, CDRH2 and CDRH3, respectively. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework.

Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions and the ability to bind specifically to the antigen. An antibody that “specifically binds to” TIGIT or PD-1 is an antibody that exhibits preferential binding to TIGIT or PD-1 (as appropriate) as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., TIGIT or PD-1). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorph (e.g., rabbits), rodent (e.g., rats or mice), Procyon lotor (e.g., raccoons). In particular embodiments, the subject is human.

The term “subject in need thereof” as used herein refers to a subject diagnosed with or suspected of having cancer or an infectious disease as defined herein.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-TIGIT antibody or an anti-PD-1 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-TIGIT antibody variant or an anti-PD-1 antibody variant) at least retains the antibody functional activity. In specific embodiments, an anti-TIGIT antibody variant binds to TIGIT and/or is antagonistic to TIGIT activity. In some embodiments, an anti-PD-1 antibody variant binds to PD-1 and/or is antagonistic to PD-1 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

As used herein, the term “treat” or “treating” means to administer a therapeutic combination of an anti-human PD-1 monoclonal antibody or antigen binding fragment thereof, an anti-human TIGIT monoclonal antibody or antigen binding fragment thereof, and lenvatinib or a pharmaceutically acceptable salt thereof, such as, e.g., an anti-human PD-1 monoclonal antibody or antigen binding fragment thereof, an anti-human TIGIT monoclonal antibody or antigen binding fragment thereof, and lenvatinib or a pharmaceutically acceptable salt thereof, to a subject or patient having one or more disease symptoms as provided herein. Typically, the agents of the therapeutic combination are administered in an amount effective to alleviate one or more disease symptoms in the treated subject or population, whether by inducing the regression of or inhibiting the progression of such symptom(s) by any clinically measurable degree. The amount of the agents of the therapeutic combination that is effective to alleviate any particular disease symptom may vary according to factors such as the disease state, age, and weight of the patient, and the ability of the therapeutic combination to elicit a desired response in the subject. Whether a disease symptom has been alleviated can be assessed by any clinical measurement typically used by physicians or other skilled healthcare providers to assess the severity or progression status of that symptom.

“Treat” or “treating” cancer as used herein means to administer a therapeutic combination of an anti-human PD-1 monoclonal antibody or antigen binding fragment thereof, an anti-human TIGIT monoclonal antibody or antigen binding fragment thereof, and lenvatinib or a pharmaceutically acceptable salt thereof, such as, e.g., an anti-human PD-1 monoclonal antibody or antigen binding fragment thereof, an anti-human TIGIT monoclonal antibody or antigen binding fragment thereof, and an lenvatinib or a pharmaceutically acceptable salt thereof, to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth, comprising administration by oral, mucosal, intradermal, intravenous, subcutaneous, intramuscular delivery, and/or any other methods of physical delivery described herein or known in the art. “Treatment” may include one or more of the following: inducing/increasing an antitumor immune response, decreasing the number of one or more tumor markers, halting or delaying the growth of a tumor or blood cancer or progression of disease such as cancer, stabilization of disease, inhibiting the growth or survival of tumor cells, eliminating or reducing the size of one or more cancerous lesions or tumors, decreasing the level of one or more tumor markers, ameliorating or abrogating the clinical manifestations of disease, reducing the severity or duration of the clinical symptoms, prolonging the survival or patient relative to the expected survival in a similar untreated patient, and inducing complete or partial remission of a cancerous condition, wherein the disease is cancer, and in certain embodiments wherein the cancer is selected from the group consisting of endometrial cancer cervical cancer, head and neck cancer (HNSCC), biliary cancer, esophageal cancer, bladder cancer, breast cancer, triple negative breast cancer (TNBC), non-small cell lung cancer (NSCLC), colorectal cancer (CRC), renal cell carcinoma (RCC), hepatocellular carcinoma (HCC), and melanoma. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

Positive therapeutic effects in cancer can be measured in a number of ways (See, W. A. Weber, J. Nucl. Med. 50:1 S-10 S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treat” or “treating” an infectious disease or an infection as used herein means to administer a therapeutic combination of an anti-human PD-1 monoclonal antibody or antigen binding fragment thereof, an anti-human TIGIT monoclonal antibody or antigen binding fragment thereof, and lenvatinib or a pharmaceutically acceptable salt thereof, such as, e.g., an anti-human PD-1 monoclonal antibody or antigen binding fragment thereof, an anti-human TIGIT monoclonal antibody or antigen binding fragment thereof, and lenvatinib or a pharmaceutically acceptable salt thereof, to a subject having an infectious disease or an infection (e.g., caused by many pathogens, including bacteria, viruses, fungi) to achieve at least one positive therapeutic effect.

The term “co-formulation” refers to a formulation comprising two or more of therapeutic agents. In some embodiments, co-formulation comprises a TIGIT antagonist and a PD-1 antagonist.

The term “pharmaceutically acceptable carrier” refers to any inactive substance that is suitable for use in a formulation for the delivery of a therapeutic agent. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

As used herein, the terms “combination,” “combination therapy,” and “therapeutic combination” refer to treatments in which at least one anti-human TIGIT monoclonal antibody or antigen-binding fragment thereof, at least one anti-human PD-1 monoclonal antibody or antigen-binding fragment thereof, and lenvatinib or a pharmaceutically acceptable salt thereof, such as, e.g., an anti-human PD-1 monoclonal antibody or antigen binding fragment thereof, an anti-human TIGIT monoclonal antibody or antigen binding fragment thereof, and lenvatinib or a pharmaceutically acceptable salt thereof, and optionally additional therapeutic agents, each are administered to a patient in a coordinated manner, over an overlapping period of time. The period of treatment with the at least one anti-human TIGIT monoclonal antibody (or antigen-binding fragment thereof) (the “anti-TIGIT treatment”) is the period of time that a patient undergoes treatment with the anti-human TIGIT monoclonal antibody (or antigen-binding fragment thereof); that is, the period of time from the initial dosing with the anti-human TIGIT monoclonal antibody (or antigen-binding fragment thereof) through the final day of a treatment cycle. Similarly, the period of treatment with the at least one anti-human PD-1 monoclonal antibody (or antigen-binding fragment thereof) (the “anti-PD-1 treatment”) is the period of time that a patient undergoes treatment with the anti-human PD-1 monoclonal antibody (or antigen-binding fragment thereof); that is, the period of time from the initial dosing with the anti-human PD-1 monoclonal antibody (or antigen-binding fragment thereof) through the final day of a treatment cycle. The period of treatment with lenvatinib or a pharmaceutically acceptable salt thereof (the “lenvatinib treatment”) is the period of time that a patient undergoes treatment with lenvatinib; that is, the period of time from the initial dosing with lenvatinib through the final day of a treatment cycle. In the methods, uses, and therapeutic combinations described herein, the anti-TIGIT treatment overlaps by at least one day with the anti-PD-1 treatment and overlaps by at least one day with the lenvatinib treatment. In certain embodiments, the anti-TIGIT treatment, the anti-PD-1 treatment, and the lenvatinib treatment are the same period of time. In some embodiments, the anti-TIGIT treatment begins prior to the anti-PD-1 and/or the lenvatinib treatment. In other embodiments, the anti-TIGIT treatment begins after the anti-PD-1 and/or the lenvatinib treatment. In yet other embodiments, the anti-PD-1 treatment begins prior to the anti-TIGIT and/or the lenvatinib treatment. In still other embodiments, the anti-PD-1 treatment begins after the anti-TIGIT and/or the lenvatinib treatment. In some embodiments, the lenvatinib treatment begins prior to the anti-PD-1 and/or the anti-TIGIT treatment. In other embodiments, the lenvatinib treatment begins after the anti-PD-1 and/or the anti-TIGIT treatment. In certain embodiments, the anti-TIGIT treatment is terminated prior to termination of the anti-PD-1 and/or the lenvatinib treatment. In other embodiments, the anti-TIGIT treatment is terminated after termination of the anti-PD-1 and/or the lenvatinib treatment. In yet other embodiments, the anti-PD-1 treatment is terminated prior to termination of the anti-TIGIT and/or the lenvatinib treatment. In still other embodiments, the anti-PD-1 treatment is terminated after termination of the anti-TIGIT and/or the lenvatinib treatment. In certain embodiments, the lenvatinib treatment is terminated prior to termination of the anti-PD-1 and/or the anti-TIGIT treatment. In other embodiments, the lenvatinib treatment is terminated after termination of the anti-PD-1 and/or the anti-TIGIT treatment.

The terms “treatment regimen,” “dosing protocol,” and “dosing regimen” are used interchangeably to refer to the dose and timing of administration of each therapeutic agent in a combination therapy of the disclosure.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include, but are not limited to, squamous cell carcinoma, myeloma, small-cell lung cancer, non-small cell lung cancer, glioma, Hodgkin lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, hepatocellular carcinoma, biliary cancer, esophageal cancer, breast cancer, triple negative breast cancer, colon carcinoma, and head and neck cancer.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AML)), lymphoma (such as DLBCL), multiple myeloma (MM), etc.).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

As used herein, the term “effective amount” refer to an amount of an anti-TIGIT antibody or antigen binding fragment, an anti-PD-1 antibody or antigen binding fragment of the invention, and lenvatinib or a pharmaceutically acceptable salt thereof, such as, e.g., an anti-human PD-1 monoclonal antibody or antigen binding fragment thereof, an anti-human TIGIT monoclonal antibody or antigen binding fragment thereof, and lenvatinib or a pharmaceutically acceptable salt thereof, that, when administered alone or in combination with an additional therapeutic agent to a cell, tissue, or subject, is effective to cause a measurable improvement in one or more symptoms of an infection or a disease, for example cancer or the progression of cancer. An effective amount further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic may result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. Toxicity and therapeutic efficacy of the antibodies or antigen binding fragments of the invention, administered alone or in combination with another therapeutic agent, can be determined by any number of systems or means. For example, the toxicity and therapeutic efficacy of the antibodies or antigen binding fragments or compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

It is understood that wherever embodiments are described herein with the language “comprising,” otherwise analogous embodiments described in terms of “consisting of” and/or “consisting essentially of” are also provided.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

### Abbreviations with Expanded Term

TABLE 3
Abbreviations

| Abbreviation | Expanded Term |
|---|---|
| 1L | First line |
| ADA | antidrug antibody |
| AE | adverse event |
| AJCC | American Joint Committee on Cancer |
| ALT | Alanine aminotransferase |
| APaT | All-Participants-as-Treated |
| aPTT | Activated partial thromboplastin time |
| BCG | Bacillus Calmette-Guerin |
| BICR | blinded independent central review |
| BP | blood pressure |
| BRAF | v-raf murine sarcoma viral oncogene homolog B1 |
| C1 | Cycle 1 |
| C2 | Cycle 2 |
| C3 | Cycle 3 |
| C4 | Cycle 4 |
| C5 | Cycle 5 |
| C6 | Cycle 6 |
| C10 | Cycle 10 |
| C14 | Cycle 14 |
| C1D1 | Cycle 1 Day 1 |
| CD | cluster of differentiation |
| CDR | Complementary determining region |
| CD28 | cluster of differentiation 28 |
| CHO | Chinese hamster ovary |
| CIV | central imaging vendor |
| CNS | central nervous system |
| CR | complete response |
| CSR | Clinical Study Report |
| CT | computed tomography |
| CTCAE | Common Terminology Criteria for Adverse Events |
| CTCAE 5.0 | Common Terminology Criteria for |
| | Adverse Events, version 5.0 |
| D1 | Day 1 |
| D8 | Day 8 |
| D15 | Day 15 |
| D/C | discontinue/discontinuing |
| DLT | dose-limiting toxicity |
| DNA | deoxyribonucleic acid |
| DOR | Duration of Response |
| ECG | Electrocardiogram |
| ECHO | Echocardiogram |
| ECI | event of clinical interest |
| ECOG | Eastern Cooperative Oncology Group |
| EFS | event-free survival |
| EOT | End of treatment |
| EU | European Union |
| FR | framework region |
| HBsAg | Hepatitis B surface antigen |
| HBV | Hepatitis B virus |
| HCV | Hepatitis C virus |
| HIV | human immunodeficiency virus |
| IA | interim analysis |
| IB | Investigator's Brochure |
| IC50 | mean inhibitory concentration |
| ICF | Informed Consent Form |
| iCPD | iRECIST confirmed progressive disease |
| iCR | iRECIST complete response |
| IgG | Immunoglobulin G |
| IgG1 | Immunoglobulin G, subclass 1 |
| IMP | Investigational medicinal product |
| IND | Investigational New Drug |
| IO | immuno-oncology |
| IP | Intraperitoneal |
| iPR | iRECIST partial response |
| irAE | immune-related adverse event |
| iRECIST | Response Evaluation Criteria in Solid Tumors |
| | 1.1 for immune-based therapeutics |
| iSD | iRECIST stable disease |
| IT | Intratumoral |
| ITT | immunoglobulin tail tyrosine |
| ITIM | immunoreceptor tyrosine-based inhibitory motif |
| iUPD | iRECIST unconfirmed progressive disease |
| IV | Intravenous |
| IVD | in vitro diagnostic |
| kg | Kilogram |
| LVEF | left ventricular ejection fraction |
| mAb | monoclonal antibody |
| MedDRA | Medical Dictionary for Regulatory Activities |
| Mg | Milligram |
| mL | Milliliter |
| Mm | Millimeter |
| mm³ | cubic millimeter, 0.001 mL |
| MPK | milligram per kilogram |
| MRI | magnetic resonance imaging |
| MSD | Merck Sharp and Dohme Corp., |
| | a subsidiary of Merck & Co., Inc. |
| MSI | microsatellite instability |
| mTPI | modified Toxicity Probability Interval |
| MUGA | multiple-gated acquisition |
| N | number of subjects in a treatment group |
| NCI | National Cancer Institute |
| NE | not estimable |
| NK | Natural killer (cells) |
| NK T | Natural killer T (cells) |
| NSCLC | non-small cell lung cancer |
| OR | overall response |
| ORR | objective response rate |
| OS | overall survival |
| PBPK | physiologically-based PK |
| PD-1 | programmed cell-death 1 |
| PDGFR | platelet-derived growth factor receptor |
| PD-L1 | programmed cell-death ligand 1 |
| PD-L2 | programmed cell-death ligand 2 |
| PE | Physical examination |
| PET | positron emission tomography |
| PFS | Progression-free survival |
| PK | Pharmacokinetic |
| PO | by mouth (orally) |
| PP | Per Protocol |
| pPR | pathological partial response |
| PR | partial response |
| PRO | patient-reported outcome |
| pRR | pathological response rate |
| PT | prothrombin time |
| PTT | partial thromboplastin time |
| Q2W | every 2 weeks |
| Q3W | every 3 weeks |
| Q6W | every 6 weeks |
| Q9W | every 9 weeks |
| Q12W | every 12 weeks |
| Q24W | every 24 weeks |
| QD | once daily / one dose per day |
| Q5D | dose every 5 days |
| Q7D | dose every 7 days |
| Q10D | dose every 10 days |
| QoL | quality of life |
| QTc | The QTc interval is the length of time |
| | it take the electrical system in the |
| | heart to repolarize, adjusted for heart rate. |
| RCC | renal cell carcinoma |
| RECIST | Response Evaluation Criteria in Solid Tumors |
| RECIST 1.1 | Response Evaluation Criteria in Solid Tumors 1.1 |
| RENCA | Renal cell carcinoma |
| RNA | ribonucleic acid |
| RP2D | recommended Phase 2 dose |
| RR | Respiratory rate |
| SAE | serious adverse event |
| SD | stable disease |
| SEM | standard error of the mean |
| SoA | Schedule of Activities |
| T3 | Triiodothyronine |
| T/C | median tumor volume of the treated |
| | animal/Median tumor volume of the |
| | control animal |
| TIGIT | T cell immunoreceptor with Ig and ITIM domains |
| TSH | thyroid-stimulating hormone |
| μg, ug, UG | Microgram |
| US | United States |
| VEGF | vascular endothelial growth factor |
| VEGFR | vascular endothelial growth factor receptors |
| V_{H} | immunoglobulin heavy chain variable region |
| V_{K} | immunoglobulin kappa light chain variable region |
| V_{L} | immunoglobulin light chain variable region |
| WOCBP | woman/women of childbearing potential |

### PD-1 Antagonists

Provided herein are PD-1 antagonists or anti-human PD-1 monoclonal antibodies that can be used in any of the methods, compositions, kits, and uses disclosed herein, including any chemical compound or biological molecule that blocks binding of PD-L1 to PD-1 and preferably also blocks binding of PD-L2 to PD-1.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, pidilizumab (U.S. Pat. No. 7,332,582), AMP-514 (MedImmune LLC, Gaithersburg, MD), PDR001 (U.S. Pat. No. 9,683,048), BGB-A317 (U.S. Pat. No. 8,735,553), and MGA012 (MacroGenics, Rockville, MD). In one embodiment, the anti-human PD-1 monoclonal antibody is pembrolizumab. In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, an anti-human PD-1 antibody or antigen binding fragment thereof for use in the methods, kits, uses and co-formulations of the invention comprises three light chain CDRs of CDRL1, CDRL2 and CDRL3 and/or three heavy chain CDRs of CDRH1, CDRH2 and CDRH3.

In one embodiment of the invention, CDRL1 has the amino acid sequence as set forth in SEQ ID NO:1 or a variant of the amino acid sequence as set forth in SEQ ID NO:1, CDRL2 has the amino acid sequence as set forth in SEQ ID NO:2 or a variant of the amino acid sequence as set forth in SEQ ID NO:2, and CDRL3 has the amino acid sequence as set forth in SEQ ID NO:3 or a variant of the amino acid sequence as set forth in SEQ ID NO:3.

In one embodiment, CDRH1 has the amino acid sequence as set forth in SEQ ID NO:6 or a variant of the amino acid sequence as set forth in SEQ ID NO:6, CDRH2 has the amino acid sequence as set forth in SEQ ID NO: 7 or a variant of the amino acid sequence as set forth in SEQ ID NO:7, and CDRH3 has the amino acid sequence as set forth in SEQ ID NO:8 or a variant of the amino acid sequence as set forth in SEQ ID NO:8.

In one embodiment, the three light chain CDRs have the amino acid sequences as set forth in SEQ ID NO:1, SEQ ID NO:2, and SEQ ID NO:3 and the three heavy chain CDRs have the amino acid sequences as set forth in SEQ ID NO:6, SEQ ID NO:7 and SEQ ID NO:8.

In an alternative embodiment of the invention, CDRL1 has the amino acid sequence as set forth in SEQ ID NO:11 or a variant of the amino acid sequence as set forth in SEQ ID NO:11, CDRL2 has the amino acid sequence as set forth in SEQ ID NO:12 or a variant of the amino acid sequence as set forth in SEQ ID NO: 12, and CDRL3 has the amino acid sequence as set forth in SEQ ID NO:13 or a variant of the amino acid sequence as set forth in SEQ ID NO:13.

In one embodiment, CDRH1 has the amino acid sequence as set forth in SEQ ID NO:16 or a variant of the amino acid sequence as set forth in SEQ ID NO:16, CDRH2 has the amino acid sequence as set forth in SEQ ID NO:17 or a variant of the amino acid sequence as set forth in SEQ ID NO:17, and CDRH3 has the amino acid sequence as set forth in SEQ ID NO:18 or a variant of the amino acid sequence as set forth in SEQ ID NO:18.

In one embodiment, the three light chain CDRs have the amino acid sequences as set forth in SEQ ID NO:1, SEQ ID NO:2, and SEQ ID NO:3 and the three heavy chain CDRs have the amino acid sequences as set forth in SEQ ID NO:6, SEQ ID NO:7 and SEQ ID NO:8.

In an alternative embodiment, the three light chain CDRs have the amino acid sequences as set forth in SEQ ID NO:11, SEQ ID NO:12, and SEQ ID NO:13 and the three heavy chain CDRs have the amino acid sequences as set forth in SEQ ID NO:16, SEQ ID NO:17 and SEQ ID NO:18.

In a further embodiment of the invention, CDRL1 has the amino acid sequence as set forth in SEQ ID NO:21 or a variant of the amino acid sequence as set forth in SEQ ID NO:21, CDRL2 has the amino acid sequence as set forth in SEQ ID NO:22 or a variant of the amino acid sequence as set forth in SEQ ID NO:22, and CDRL3 has the amino acid sequence as set forth in SEQ ID NO:23 or a variant of the amino acid sequence as set forth in SEQ ID NO:23.

In yet another embodiment, CDRH1 has the amino acid sequence as set forth in SEQ ID NO:24 or a variant of the amino acid sequence as set forth in SEQ ID NO:24, CDRH2 has the amino acid sequence as set forth in SEQ ID NO: 25 or a variant of the amino acid sequence as set forth in SEQ ID NO:25, and CDRH3 has the amino acid sequence as set forth in SEQ ID NO:26 or a variant of the amino acid sequence as set forth in SEQ ID NO:26.

In another embodiment, the three light chain CDRs have the amino acid sequences as set forth in SEQ ID NO:21, SEQ ID NO:22, and SEQ ID NO:23 and the three heavy chain CDRs have the amino acid sequences as set forth in SEQ ID NO:24, SEQ ID NO:25 and SEQ ID NO:26.

Some anti-human PD-1 antibody and antigen binding fragments of the invention comprise a light chain variable region and a heavy chain variable region. In some embodiments, the light chain variable region comprises the amino acid sequence as set forth in SEQ ID NO:4 or a variant of the amino acid sequence as set forth in SEQ ID NO:4, and the heavy chain variable region comprises the amino acid sequence as set forth in SEQ ID NO:9 or a variant of the amino acid sequence as set forth in SEQ ID NO:9. In further embodiments, the light chain variable region comprises the amino acid sequence as set forth in SEQ ID NO:14 or a variant of the amino acid sequence as set forth in SEQ ID NO:14, and the heavy chain variable region comprises the amino acid sequence as set forth in SEQ ID NO:19 or a variant of the amino acid sequence as set forth in SEQ ID NO:19. In further embodiments, the heavy chain variable region comprises the amino acid sequence as set forth in SEQ ID NO:27 or a variant of the amino acid sequence as set forth in SEQ ID NO:27 and the light chain variable region comprises the amino acid sequence as set forth in SEQ ID NO:28 or a variant of the amino acid sequence as set forth in SEQ ID NO:28, the amino acid sequence as set forth in SEQ ID NO:29 or a variant of the amino acid sequence as set forth in SEQ ID NO:29, or the amino acid sequence as set forth in SEQ ID NO:30 or a variant of the amino acid sequence as set forth in SEQ ID NO:30. In such embodiments, a light chain variable region or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the co-formulations, methods, kits or uses of the invention, the anti-human PD-1 antibody or antigen binding fragment comprises a light chain variable region comprising or consisting of the amino acid sequence as set forth in SEQ ID NO:4 and a heavy chain variable region comprising or consisting of the amino acid sequence as set forth in SEQ ID NO:9. In a further embodiment, the anti-human PD-1 antibody or antigen binding fragment comprises a light chain variable region comprising or consisting of the amino acid sequence as set forth in SEQ ID NO:14 and a heavy chain variable region comprising or consisting of the amino acid sequence as set forth in SEQ ID NO:19. In one embodiment of the formulations of the invention, the anti-human PD-1 antibody or antigen binding fragment comprises a light chain variable region comprising or consisting of the amino acid sequence as set forth in SEQ ID NO:28 and a heavy chain variable region comprising or consisting of the amino acid sequence as set forth in SEQ ID NO:27. In a further embodiment, the anti-human PD-1 antibody or antigen binding fragment comprises a light chain variable region comprising or consisting of the amino acid sequence as set forth in SEQ ID NO:29 and a heavy chain variable region comprising or consisting of the amino acid sequence as set forth in SEQ ID NO:27. In another embodiment, the antibody or antigen binding fragment comprises a light chain variable region comprising or consisting of the amino acid sequence as set forth in SEQ ID NO:30 and a heavy chain variable region comprising or consisting of the amino acid sequence as set forth in SEQ ID NO:27.

In another embodiment, the co-formulations, methods, kits or uses of the invention comprise an anti-human PD-1 antibody or antigen binding protein that has a V_{L} domain and/or a V_{H} domain with at least 95%, 90%, 85%, 80%, 75% or 50% sequence homology to one of the V_{L} domains or V_{H} domains described above, and exhibits specific binding to PD-1. In another embodiment, the anti-human PD-1 antibody or antigen binding protein of the co-formulations of the invention comprises V_{L} and V_{H} domains having up to 1, 2, 3, 4, or 5 or more amino acid substitutions, and exhibits specific binding to PD-1.

In any of the embodiments above, the PD-1 antagonist may be a full-length anti-PD-1 antibody or an antigen binding fragment thereof that specifically binds human PD-1. In certain embodiments, the PD-1 antagonist is a full-length anti-PD-1 antibody selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the PD-1 antagonist is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO: 10. In alternative embodiments, the PD-1 antagonist is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the PD-1 antagonist is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the PD-1 antagonist is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the PD-1 antagonist is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some co-formulations of the invention, the PD-1 antagonist is pembrolizumab or a pembrolizumab biosimilar. In some co-formulations of the invention, the PD-1 antagonist is nivolumab or a nivolumab biosimilar.

Ordinarily, amino acid sequence variants of the anti-PD-1 antibodies and antigen binding fragments of the invention and the anti-TIGIT antibodies and antigen binding fragments will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the anti-PD-1 residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 4
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWYQQKPGQ | 4 |
| Region | APRLLIYLASYLESGVPARFSGSGSGTDFTLTISSLEPEDFAVYYC | |
| | QHSRDLPLTFGGGTKVEIK | |
| Light | EIVLTQSPATLSLSPGERATLSCRASKGVSTSGYSYLHWYQQKPGQ | 5 |
| Chain | APRLLIYLASYLESGVPARFSGSGSGTDFTLTISSLEPEDFAVYYC | |
| | QHSRDLPLTFGGGTKVEIKRTVAAPSVFIFPPSDEQLKSGTASVVC | |
| | LLNNFYPREAKVQWKVDNALQSGNSQESVTEQDSKDSTYSLSSTLT | |
| | LSKADYEKHKVYACEVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWVRQAPGQGL | 9 |
| Region | EWMGGINPSNGGTNFNEKFKNRVTLTTDSSTTTAYMELKSLQFDD | |
| | TAVYYCARRDYRFDMGFDYWGQGTTVTVSS | |
| Heavy | QVQLVQSGVEVKKPGASVKVSCKASGYTFTNYYMYWVRQAPGQGLE | 10 |
| Chain | WMGGINPSNGGTNFNEKFKNRVTLTTDSSTTTAYMELKSLQFDDTA | |
| | VYYCARRDYRFDMGFDYWGQGTTVTVSSASTKGPSVFPLAPCSRST | |
| | SESTAALGCLVKDYFPEPVTVSWNSGALTSGVHTFPAVLQSSGLYS | |
| | LSSVVTVPSSSLGTKTYTCNVDHKPSNTKVDKRVESKYGPPCPPCP | |
| | APEFLGGPSVFLFPPKPKDTLMISRTPEVTCVVVDVSQEDPEVQFN | |
| | WYVDGVEVHNAKTKPREEQFNSTYRVVSVLTVLHQDWLNGKEYKCK | |
| | VSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDK | |
| | SRWQEGNVFSCSVMHEALHNHYTQKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSYLAWYQQKPGQAPRL | 14 |
| Region | LIYDASNRATGIPARFSGSGSGTDFTLTISSLEPEDFAVYYCQQSS | |
| | NWPRTFGQGTKVEIK | |
| Light | EIVLTQSPATLSLSPGERATLSCRASQSVSSYLAWYQQKPGQAPRL | 15 |
| Chain | LIYDASNRATGIPARFSGSGSGTDFTLTISSLEPEDFAVYYCQQSS | |
| | NWPRTFGQGTKVEIKRTVAAPSVFIFPPSDEQLKSGTASVVCLLNN | |
| | FYPREAKVQWKVDNALQSGNSQESVTEQDSKDSTYSLSSTLTLSKA | |
| | DYEKHKVYACEVTHQGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNSGMHWVRQAPGKGLE | 19 |
| Region | WVAVIWYDGSKRYYADSVKGRFTISRDNSKNTLFLQMNSLRAEDTA | |
| | VYYCATNDDYWGQGTLVTVSS | |
| Heavy | QVQLVESGGGVVQPGRSLRLDCKASGITFSNSGMHWVRQAPGKGLE | 20 |
| Chain | WVAVIWYDGSKRYYADSVKGRFTISRDNSKNTLFLQMNSLRAEDTA | |
| | VYYCATNDDYWGQGTLVTVSSASTKGPSVFPLAPCSRSTSESTAAL | |
| | GCLVKDYFPEPVTVSWNSGALTSGVHTFPAVLQSSGLYSLSSVVTV | |
| | PSSSLGTKTYTCNVDHKPSNTKVDKRVESKYGPPCPPCPAPEFLGG | |
| | PSVFLFPPKPKDTLMISRTPEVTCVVVDVSQEDPEVQFNWYVDGVE | |
| | VHNAKTKPREEQFNSTYRVVSVLTVLHQDWLNGKEYKCKVSNKGLP | |
| | SSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGN | |
| | VFSCSVMHEALHNHYTQKSLSLSLGK | |

TABLE 5
Additional PD-1 Antibodies and Antigen Binding
Fragments Useful in the Co-Formulations, Compositions,
Methods, Kits and Uses of the Invention.

| A. Antibodies and antigen binding fragments comprising light | |
|---|---|
| and heavy chain CDRs of hPD-1.08A in WO2008/156712 | |
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |
| C. Antibodies and antigen binding fragments comprising | |
| the mature h109A heavy chain variable region and | |
| one of the mature K09A light chain variable regions | |
| in WO 2008/156712 | |
| Heavy chain VR | SEQ ID NO: 27 |
| Light chain VR | SEQ ID NO: 28 or SEQ ID NO: 29 |
| | or SEQ ID NO: 30 |
| D. Antibodies and antigen binding fragments | |
| comprising the mature 409 heavy chain and one of | |
| the mature K09A light chains in WO 2008/156712 | |
| Heavy chain | SEQ ID NO: 31 |
| Light chain | SEQ ID NO: 32 or SEQ ID NO: 33 |
| | or SEQ ID NO: 34 |

### TIGIT Antagonists

Provided herein are anti-human TIGIT monoclonal antibodies or antigen binding fragments thereof that can be used in the methods, pharmaceutical compositions, kits, and uses disclosed herein. Any monoclonal antibodies that bind to a TIGIT polypeptide, a TIGIT polypeptide fragment, a TIGIT peptide, or a TIGIT epitope and block the interaction between TIGIT and its ligand CD155 and/or CD112 can be used. In some embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155. In other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD112. In yet other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155 and the interaction between TIGIT and CD112. In some embodiments the human constant region is selected from the group consisting of IgG1, IgG2, IgG3 and IgG4 constant regions, and in preferred embodiments, the human constant region is an IgG1 or IgG4 constant region.

Exemplary anti-TIGIT antibody sequences are set forth below in Tables 6 and 7.

TABLE 6
Exemplary anti-TIGIT antibodies

| | SEQ | |
|---|---|---|
| | ID | |
| Description | NO: | SEQUENCE |
| 14A6 H-CDR1 | 35 | SDYWG |
| 14A6 H-CDR2 | 36 | FITYSGSTSYNPSLKS |
| 14A6 H-CDR3 | 37 | MPSFITLASLSTWEGYFDF |
| 14A6 L-CDR1 | 38 | KASQSIHKNLA |
| 14A6 L-CDR2 | 39 | YANSLQT |
| 14A6 L-CDR3 | 40 | QQYYSGWT |
| 14A6 PARENTAL VH | 41 | EVQLQESGPGLVKPSQSLSLTCSVTGSSIASDYWGWIR |
| | | KFPGNKMEWMGFITYSGSTSYNPSLKSRISITRDTSKN |
| | | QFFLQLHSVTTDDTATYSCARMPSFITLASLSTWEGYF |
| | | DFWGPGTMVTVSS |
| 14A6 PARENTAL VL | 42 | DIQMTQSPSLLSASVGDRVTLNCKASQSIHKNLAWYQQ |
| | | KLGEAPKFLIYYANSLQTGIPSRFSGSGSGTDFTLTIS |
| | | GLQPEDVATYFCQQYYSGWTFGGGTKVELK |
| Hu14A6VH.1 | 43 | EVQLQESGPGLVKPSETLSLTCTVSGGSISSDYWGWIR |
| | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| Hu14A6VH.1a | 44 | EVQLQESGPGLVKPSETLSLTCTVSGGSISSDYWGWIR |
| | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| Hu14A6VH.1b | 45 | EVQLQESGPGLVKPSETLSLTCTVSGGSISSDYWGWIR |
| | | QPPGKGLEWIGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.1c | 46 | EVQLQESGPGLVKPSETLSLTCTVSGSSISSDYWGWIR |
| | | QPPGKGLEWMGFITYSGSTSYNPSLKSRITISRDTSKN |
| | | QFSLKLSSVTAADTAVYYCARMPSFITLASLSTWEGY |
| | | FDFWGQGTMVTVSS |
| Hu14A6VH.1d | 47 | EVQLQESGPGLVKPSETLSLTCTVSGGSISSDYWGWIR |
| | | QPPGKGLEWIGFITYSGSTSYNPSLKSRVTISRDTSKN |
| | | QFSLKLHSVTAADTAVYYCARMPSFITLASLSTWEGYF |
| | | DFWGQGTMVTVSS |
| Hu14A6VH.1e | 48 | EVQLQESGPGLVKPSETLSLTCTVSGGSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.1f | 49 | EVQLQESGPGLVKPSETLSLTCTVSGSSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.1g | 50 | EVQLQESGPGLVKPSETLSLTCTVSGSSISSDYWGWIR |
| | | QPPGKGLEWMGFITYSGSTSYNPSLKSRITISVDTSKN |
| | | QFSLKLHSVTAADTAVYYCARMPSFITLASLSTWEGY |
| | | FDFWGQGTMVTVSS |
| Hu14A6VH.2 | 51 | EVQLQESGPGLVKPSETLSLTCAVSGYSISSDYWGWIR |
| | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| Hu14A6VH.2a | 52 | EVQLQESGPGLVKPSETLSLTCAVSGYSISSDYWGWIR |
| | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| Hu14A6VH.2b | 53 | EVQLQESGPGLVKPSETLSLTCAVSGYSISSDYWGWIR |
| | | QPPGKGLEWIGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.2c | 54 | EVQLQESGPGLVKPSETLSLTCAVSGSSISSDYWGWIR |
| | | QPPGKGLEWMGFITYSGSTSYNPSLKSRITISRDTSKN |
| | | QFSLKLSSVTAADTAVYYCARMPSFITLASLSTWEGY |
| | | FDFWGQGTMVTVSS |
| Hu14A6VH.2d | 55 | EVQLQESGPGLVKPSETLSLTCAVSGYSISSDYWGWIR |
| | | QPPGKGLEWIGFITYSGSTSYNPSLKSRVTISRDTSKN |
| | | QFSLKLHSVTAADTAVYYCARMPSFITLASLSTWEGYF |
| | | DFWGQGTMVTVSS |
| Hu14A6VH.2e | 56 | EVQLQESGPGLVKPSETLSLTCAVSGYSISSDYWGWIR |
| | | QPPGKGLEWIGFITYSGSTSYNPSLKSRITISRDTSKN |
| | | FSLKLHSVTAADTAVYYCARMPSFITLASLSTWEGYF |
| | | QDFWGQGTMVTVSS |
| Hu14A6VH.2f | 57 | EVQLQESGPGLVKPSETLSLTCAVSGSSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.2e | 58 | EVQLQESGPGLVKPSETLSLTCAVSGSSISSDYWGWIR |
| | | QPPGKGLEWMGFITYSGSTSYNPSLKSRITISRDTSKN |
| | | QFSLKLHSVTAADTAVYYCARMPSFITLASLSTWEGY |
| | | FDFWGQGTMVTVSS |
| Hu14A6Vk.1 | 59 | DIQMTQSPSSLSASVGDRVTITCKASQSIHKNLAWYQ |
| | | QKPGKAPKLLIYYANSLQTGVPSRFSGSGSGTDFTLT |
| | | ISSLQPEDFATYYCQQYYSGWTFGGGTKVEIK |
| Hu14A6Vk.la | 60 | DIQMTQSPSSLSASVGDRVTITCKASQSIHKNLAWYQ |
| | | QKPGKAPKFLIYYANSLQTGVPSRFSGSGSGTDFTLT |
| | | ISSLQPEDFATYYCQQYYSGWTFGGGTKVEIK |
| Hu14A6Vk.1b | 61 | DIQMTQSPSSLSASVGDRVTITCKASQSIHKNLAWYQ |
| | | QKPGKAPKFLIYYANSLQTGIPSRFSGSGSGTDFTLT |
| | | ISSLQPEDFATYYCQQYYSGWTFGGGTKVEIK |
| Hu14A6Vk.2 | 62 | DIQMTQSPSSLSASVGDRVTITCKASQSIHKNLAWYQ |
| | | QKPGKVPKLLIYYANSLQTGVPSRFSGSGSGTDFTLT |
| | | ISSLQPEDVATYYCQQYYSGWTFGGGTKVEIK |
| Hu14A6Vk.2a | 63 | DIQMTQSPSSLSASVGDRVTITCKASQSIHKNLAWYQ |
| | | QKPGKVPKFLIYYANSLQTGVPSRFSGSGSGTDFTLT |
| | | ISSLQPEDVATYYCQQYYSGWTFGGGTKVEIK |
| Hu14A6Vk.2b | 64 | DIQMTQSPSSLSASVGDRVTITCKASQSIHKNLAWYQ |
| | | QKPGKVPKFLIYYANSLQTGIPSRFSGSGSGTDFTLT |
| | | ISSLQPEDVATYYCQQYYSGWTFGGGTKVEIK |
| 16AHA_tigit_14a6_humanized_VH1 | 65 | EVQLQESGPGLVKPSETLSLTCTVSGSSIASDYWGWIR |
| LB155.14A6.G2.A8 VH1 | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSSAS |
| 18AHA_tigit_14a6_humanized_VH2 | 66 | EVQLQESGPGLVKPSETLSLTCTVSGSSIASDYWGWIR |
| LB155.14A6.G2.A8 VH2 | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 20AHA_tigit_14a6_humanized VH3 | 67 | EVQLQESGPGLVKPSETLSLTCTVSGSSIASDYWGWIR |
| LB155.14A6.G2.A8 VH3 | | KPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 21AHA_tigit_14a6_humanized VH4 | 68 | EVQLQESGPGLVKPSETLSLTCTVSGSSIASDYWGWIR |
| LB155.14A6.G2.A8 VH4 | | QPPGKKLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 19AHA tigit_14a6_humanized VH5 | 69 | EVQLQESGPGLVKPSETLSLTCTVSGSSIASDYWGWIR |
| LB155.14A6.G2.A8 VH5 | | QPPGKGMEWIGFITYSGSTSYNPSLKSRVTISRDTSKN |
| | | QFSLKLSSVTAADTAVYYCARMPSFITLASLSTWEGY |
| | | FDFWGQGTMVTVSS |
| 22AHA_tigit_14a6_humanized VH6 | 70 | EVQLQESGPGLVKPSETLSLTCTVSGSSIASDYWGWIR |
| LB155.14A6.G2.A8 VH6 | | KPPGKKMEWIGFITYSGSTSYNPSLKSRVTISRDTSKN |
| | | QFSLKLSSVTAADTAVYYCARMPSFITLASLSTWEGY |
| | | FDFWGQGTMVTVSS |
| 23AHA tigit 14a6_humanized_VH7 | 71 | EVQLQESGPGLVKPSETLSLTCTVSGSSIASDYWGWIR |
| LB155.14A6.G2.A8 VH7 | | QFPGKGLEWIGFITYSGSTSYNPSLKSRVTISRDTSKN |
| | | QFSLKLSSVTADDTAVYYCARMPSFITLASLSTWEGYF |
| | | DFWGQGTMVTVSS |
| 24AHA_tigit_14a6_humanized VH8 | 72 | EVQLQESGPGLVKPSETLSLTCTVSGSSIASDYWGWIR |
| LB155.14A6.G2.A8 VH8 | | KPPGKKMEWIGFITYSGSTSYNPSLKSRVTISVDTSKN |
| | | QFSLKLSSVTAADTAVYYCARMPSFITLASLSTWEGY |
| | | FDFWGQGTMVTVSS |
| 25AHA tigit 14a6_humanized_VH9 | 73 | EVQLQESGPGLVKPSETLSLTCSVTGSSIASDYWGWIR |
| LB155.14A6.G2.A8 VH9 | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| 26AHA_tigit_14a6_humanized VH10 | 74 | EVQLQQSGAGLLKPSETLSLTCSVTGSSIASDYWGWIR |
| LB155.14A6.G2.A8 VH10 | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| 27AHA_tigit_14a6_humanized_VH11 | 75 | EVQLQESGPGLVKPPGTLSLTCSVTGSSIASDYWGWVR |
| LB155.14A6.G2.A8 VH11 | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| 09AHA_tigit_14a6_humanized VL1 | 76 | DIQMTQSPSSLSASVGDRVTITCKASQSIHKNLAWYQ |
| LB155.14A6.G2.A8 VL1 | | QKPGKAPKLLIYYANSLQTGVPSRFSGSGSGTDFTLT |
| | | ISSLQPEDFATYYCQQYYSGWTFGGGTKVEIK |
| 11AHA_tigit_14a6_humanized VL2 | 77 | DIQMTQSPSSLSASVGDRVTITCKASQSIHKNLAWYQ |
| LB155.14A6.G2.A8 VL2 | | QKPGKAPKFLIYYANSLQTGVPSRFSGSGSGTDFTLT |
| | | ISSLQPEDFATYYCQQYYSGWTFGGGTKVEIK |
| 12AHA_tigit_14a6_humanized VL3 | 78 | DIQMTQSPSSLSASVGDRVTITCKASQSIHKNLAWYQ |
| LB155.14A6.G2.A8 VL3 | | QKPGKAPKLLIYYANSLQTGVPSRFSGSGSGTDFTLT |
| | | ISSLQPEDFATYFCQQYYSGWTFGGGTKVEIK |
| 13AHA tigit_14a6_humanized VL4 | 79 | DIQMTQSPSSLSASVGDRVTITCKASQSIHKNLAWYQ |
| LB155.14A6.G2.A8 VL4 | | QKPGKAPKFLIYYANSLQTGVPSRFSGSGSGTDFTLT |
| | | ISSLQPEDFATYFCQQYYSGWTFGGGTKVEIK |
| 15AHA tigit 14a6_humanized VL5 | 80 | DIQMTQSPSSLSASVGDRVTITCKASQSIHKNLAWYQQ |
| LB155.14A6.G2.A8 VL5 | | KPGKAPKLLIYYANSLQTGIPSRFSGSGSGTDFTLTIS |
| | | SLQPEDFATYYCQQYYSGWTFGGGTKVEIK |
| 28H5 H-CDR1 | 81 | GYSITSDYAWN |
| 28H5 H-CDR2 | 82 | YISNSGSASYNPSLKS |
| 28H5 H-CDR3 | 83 | LIYYDYGGAMNF |
| 28H5 L-CDR1 | 84 | KASQGVSTTVA |
| 28H5 L-CDR2 | 85 | SASYRYT |
| 28H5 L-CDR3 | 86 | QHYYSTPWT |
| 28H5 PARENTAL VH | 87 | DVQLQESGPGLVKPSQSLSLTCTVTGYSITSDYAWNWI |
| | | RQFPGNKLEWMGYISNSGSASYNPSLKSRISITRDTSK |
| | | NQFFLQLNSVTTEDTATYYCATLIYYDYGGAMNFWG |
| | | QGTSVTVSS |
| 28H5 PARENTAL VL | 88 | DIVMTQSHKFMSTSVGDRVSITCKASQGVSTTVAWYQ |
| | | QKPGQSPKLLIYSASYRYTGVPDRFTGSGSGTDFTFT |
| | | ISSVQSEDLAVYYCQHYYSTPWTFGGGTKLEIK |
| 14H6 L-CDR2 variant | 89 | YASNLQT |
| 14H6 L-CDR2 variant | 90 | YASSLQT |
| 14H6 L-CDR2 variant | 91 | YASTLQT |
| 14H6 L-CDR2 variant | 92 | YATTLQT |
| 14H6 L-CDR2 variant | 93 | YASYLQT |
| 14H6 L-CDR2 variant | 94 | YANQLQT |
| 14H6 L-CDR2 variant | 95 | YAGSLQT |
| 14H6 L-CDR2 variant | 96 | YASQLQT |
| 14H6 L-CDR2 variant | 97 | YADSLQT |
| 14H6 L-CDR3 variant | 98 | QQYYSGFT |
| 14H6 L-CDR3 variant | 99 | QQYYSGYT |
| 14H6 L-CDR3 variant | 100 | QQYYSGIT |
| 14H6 L-CDR3 variant | 101 | QQYYSGVT |
| 14H6 L-CDR3 variant | 102 | QQYYSGLT |
| 14H6 H-CDR3 variant | 103 | MPSFITLASLSTFEGYFDF |
| 14H6 H-CDR3 variant | 104 | MPSFITLASLSTYEGYFDF |
| 14H6 H-CDR3 variant | 105 | MPSFITLASLSTIEGYFDF |
| 14H6 H-CDR3 variant | 106 | MPSFITLASLSTVEGYFDF |
| 14H6 H-CDR3 variant | 107 | MPSFITLASLSTLEGYFDF |
| 31C6 H-CDR1 | 108 | SYVMH |
| 31C6 H-CDR2 | 109 | YIDPYNDGAKYNEKFKG |
| 31C6 H-CDR3 | 110 | GGPYGWYFDV |
| 31C6 L-CDR1 | 111 | RASEHIYSYLS |
| 31C6 L-CDR2 | 112 | NAKTLAE |
| 31C6 L-CDR3 | 113 | QHHFGSPLT |
| 31C6 PARENTAL VH | 114 | EVQLQQSGPELVKPGSSVKMSCKASGYTFSSYVMHW |
| | | VKQKPGQGLEWIGYIDPYNDGAKYNEKFKGKATLTS |
| | | DKSSSTAYMELSSLTSEDSAVYYCARGGPYGWYFDV |
| | | WGAGTTVTVSS |
| 31C6 PARENTAL VL | 115 | DIQMTQSPASLSASVGETVTITCRASEHIYSYLSWY |
| | | QQKQGKSPQLLVYNAKTLAEGVPSRFSGSGSGTQFS |
| | | LKINSLQPEDFGTYYCQHHFGSPLTFGAGTTLELK |
| 31C6 H-CDR2 VARIANT (D56R) | 116 | YIDPYNrGAKYNEKFG |
| 31C6 H-CDR2 VARIANT (D56L) | 117 | YIDPYNlGAKYNEKGF |
| 31C6 H-CDR2 VARIANT (D56K) | 118 | YIDPYNkGAKYNEKFG |
| 31C6 H-CDR2 VARIANT (D56F) | 119 | YIDPYNfGAKYNEKFG |
| 31C6 H-CDR2 VARIANT (D56S) | 120 | YIDPYNsGAKYNEKFG |
| 31C6 H-CDR2 VARIANT (D56Y) | 121 | YIDPYNyGAKYNEKFG |
| 31C6 H-CDR2 VARIANT (D56V) | 122 | YIDPYNvGAKYNEKFG |
| 31C6 H-CDR2 VARIANT (G57R) | 123 | YIDPYNDrAKYNEKFKG |
| 31C6 H-CDR2 VARIANT (G57N) | 124 | YIDPYNDnAKYNEKFKG |
| 31C6 H-CDR2 VARIANT (G57Q) | 125 | YIDPYNDqAKYNEKFKG |
| 31C6 H-CDR2 VARIANT (G57E) | 126 | YIDPYNDeAKYNEKFKG |
| 31C6 H-CDR2 VARIANT (G57L) | 127 | YIDPYNDlAKYNEKFKG |
| 31C6 H-CDR2 VARIANT (G57K) | 128 | YIDPYNDkAKYNEKFKG |
| 31C6 H-CDR2 VARIANT (G57S) | 129 | YIDPYNDsAKYNEKFKG |
| 31C6 H-CDR2 VARIANT (G57Y) | 130 | YIDPYNDyAKYNEKFKG |
| 31C6 H-CDR2 VARIANT (G57V) | 131 | YIDPYNDvAKYNEKFKG |
| 31C6 L-CDR2 variant (N50A) | 132 | AAKTLAE |
| 31C6 L-CDR2 variant (N50Y) | 133 | YAKTLAE |
| 31C6 L-CDR2 variant (N50W) | 134 | WAKTLAE |
| 31C6 L-CDR2 variant (N50S) | 135 | SAKTLAE |
| 31C6 L-CDR2 variant (N50T) | 136 | TAKTLAE |
| 31C6 L-CDR2 variant (N50I) | 137 | IAKTLAE |
| 31C6 L-CDR2 variant (N50V) | 138 | VAKTLAE |
| 31C6 L-CDR2 variant (A51N) | 139 | NNKTLAE |
| 31C6 L-CDR2 variant (A51I) | 140 | NIKTLAE |
| 31C6 L-CDR2 variant (A51L) | 141 | NLLTLAE |
| 31C6 L-CDR2 variant (A51T) | 142 | NTKTLAE |
| 31C6 L-CDR2 variant (A51V) | 143 | NVKTLAE |
| 31C6 HUMZ_VH1 | 144 | EVQLVQSGAEVKKPGASVKVSCKASGYTFSSYVMH |
| | | WVRQAPGQRLEWIGYIDPYNDGAKYSQKFQGRVTLT |
| | | RDTSASTAYMELSSLRSEDTAVYYCARGGPYGWYFD |
| | | VWGQGTTVTVSS |
| 31C6 HUMZ_VH2 | 145 | EVQLVQSGAEVKKPGASVKVSCKASGYTFSSYVMH |
| | | WVRQAPGQRLEWIGYIDPYNDGAKYSQKFQGRVTLT |
| | | SDKSASTAYMELSSLRSEDTAVYYCARGGPYGWYFD |
| | | VWGQGTTVTVSS |
| 31C6 HUMZ_VH3 | 146 | EVQLVQSGAEVKKPGASVKVSCKASGYTFSSYVMH |
| | | WVRQAPGQGLEWIGYIDPYNDGAKYAQKFQGRVTLT |
| | | RDTSTSTVYMELSSLRSEDTAVYYCARGGPYGWYFD |
| | | VWGQGTTVTVSS |
| 31C6 HUMZ_VH4 | 147 | EVQLVQSGAEVKKPGASVKVSCKASGYTFSSYVMH |
| | | WVRQAPGQGLEWIGYIDPYNDGAKYAQKFQGRVTLT |
| | | SDKSTSTVYMELSSLRSEDTAVYYCARGGPYGWYFD |
| | | VWGQGTTVTVSS |
| 31C6 HUMZ_VH5 | 148 | EVQLVQSGAEVKKPGSSVKVSCKASGYTFSSYVMHW |
| | | VRQAPGQGLEWIGYIDPYNDGAKYAQKFQGRVTLTS |
| | | DKSTSTAYMELSSLRSEDTAVYYCARGGPYGWYFDV |
| | | WGQGTTVTVSS |
| 31C6_HUMZ_VH6 | 149 | EVQLVQSGAEVKKPGASVKVSCKASGYTFSSYVMH |
| | | WVRQAPGQGLEWIGYIDPYNDGAKYAQKFQGRVTLT |
| | | SDKSISTAYMELSRLRSDDTVVYYCARGGPYGWYFD |
| | | VWGQGTTVTVSS |
| 31C6_Humz_L1 | 150 | DIQMTQSPSSLSASVGDRVTITCRASEHIYSYLSWY |
| | | QQKPGKAPKLLIYNAKTLAEGVPSRFSGSGSGTDFT |
| | | LTISSLQPEDFATYYCQHHFGSPLTFGQGTRLEIK |
| 31C6_Humz_L2 | 151 | DIQMTQSPSSLSASVGDRVTITCRASEHIYSYLSWY |
| | | QQKPGKAPKLLIYNAKTLAEGVPSRFSGSGSGTQFT |
| | | LTISSLQPEDFATYYCQHHFGSPLTFGQGTRLEIK |
| 31C6 Humz_L3 | 152 | DIQMTQSPSSLSASVGDRVTITCRASEHIYSYLSWY |
| | | QQKPGKVPKLLIYNAKTLAEGVPSRFSGSGSGTDFT |
| | | LTISSLQPEDVATYYCQHHFGSPLTFGQGTRLEIK |
| 31C6_Humz_L4 | 153 | DIQMTQSPSSLSASVGDRVTITCRASEHIYSYLSWY |
| | | QQKPGKVPKLLIYNAKTLAEGVPSRFSGSGSGTQFT |
| | | LTISSLQPEDVATYYCQHHFGSPLTFGQGTRLEIK |
| 31C6 H-CDR2 variant | 154 | YIDPYNDGAKYAQKFQG |
| 31C6 H-CDR2 variant | 155 | YIDPYNDGAKYSQKFQG |
| 18G10-VH sequence | 156 | QVQLMESGPGLVQPSQTLSLTCTVSGFPLTSYTVHWV |
| | | RQPPGKGLEWIGAIWSSGSTDYNSALKSRLNINRDSS |
| | | KSQVFLKMNSLQTEDTAIYFCTKSGWAFFDYWGQGVM |
| | | VTVSS |
| 18G10-VL sequence | 157 | DIQMTQSPSLLSASVGDRVTLNCIASQNIYKSLAWYQ |
| | | LKLGEAPKLLIYNANSLQAGIPSRFSGSGSGTDFALT |
| | | ISGLQPEDVATYFCQQYSGGYTFGAGTKLELK |
| 11A11-VH sequence | 158 | EVQLVESGGDLVQPGRSLKISCVASGFTFSDYYMAWV |
| | | RLAPQKGLEWVASISYEGSRTHYGDSVRGRFIISRDN |
| | | PKNILYLQMNSLGSEDTATYFCARHTGTLDWLVYWG |
| | | QGTLVIVSS |
| 11A11-VL sequence | 159 | NIVMAQSPKSMSISAGDRVTMNCKASQNVDNNIAWY |
| | | QQKPGQSPKLLIFYASNRYSGVPDRFTGGGYGTDFT |
| | | LTIKSVQAEDAAFYYCQRIYNFPTFGSGTKLEIK |
| 14A6 H-CDR3 | 160 | MPSFITLASLSTXEGYFDF |
| CONSENSUS | | X = W, F, Y, I, V, L |
| 14A6 L-CDR2 | 161 | YAX₁X₂LQT |
| CONSENSUS | | X₁ = N, S, T, G, D |
| | | X₂ = S, N, S, T, Y, Q |
| 14A6 L-CDR3 | 162 | QQYYSGXT |
| CONSENSUS | | X = W, F, Y, I, V, L |
| 14A6 VH | 163 | EVQLQX₁SGX₂GLX₃KPX₄X₅X₆LSLTCX₇VX₈GX₃₀SIX₃₁S |
| PARENTAL | | DYWGWX₉RX₁₀X₁₁PGX₁₂X₁₃X₁₄EWX₁₅GFITYSGSTSYN |
| CONSENSUS | | PSLKSRX₁₆X₁₇1X₁₈X₁₉DTSKNQFX₂₀LX₂₁LX₂₂SVTX₂₃ |
| | | X₂₄DTAX₂₅YX₂₆CARMPSFITLASLSTX₂₇EGYFDFWGX₃₂ |
| | | GTX₂₈X₂₉TVSS |
| | | X₁ = E or Q |
| | | X₂ = P or A |
| | | X₃ = V or L |
| | | X₄ = S or P |
| | | X₅ = Q or E or G |
| | | X₆ = S or T |
| | | X₇ = S or T or A |
| | | X₈ = T or S |
| | | X₉ = I or V |
| | | X₁₀ = K or Q |
| | | X₁₁ = F or P |
| | | X₁₂ = N or K |
| | | X₁₃ = K or G |
| | | X₁₄ = M or L |
| | | X₁₅ = M or I |
| | | X₁₆ = I or V |
| | | X₁₇ = S or T |
| | | X₁₈ = T or S |
| | | X₁₉ = R or V |
| | | X₂₀ = F or S |
| | | X₂₁ = Q or K |
| | | X₂₂ = H or S |
| | | X₂₃ = T or A |
| | | X₂₄ = D or A |
| | | X₂₅ = T or V |
| | | X₂₆ = S or Y, |
| | | X₂₇ = W, F, Y, I, V or L |
| | | X₂₈ = M, V, L, A, R, N, P, Q, |
| | | E, G, I, H, K, F, S, T, W or Y |
| | | X₂₉ = V, T or L |
| | | X₃₀ = S or G or Y |
| | | X₃₁ = A or S |
| | | X₃₂ = P or Q |
| 14A6 VH HUMANIZED | 164 | EVQLQX₁SGX₂GLX₃KPX₄X₅TLSLTCX₆VX₇GX₈SIX₉SD |
| CONSENSUS | | YWGWX₁₀RX₁₁X₁₂PGKX₁₃X₁₄EWX₁₅GFITYSGSTSYNP |
| | | SLKSRX₁₆TISX₁₇DTSKNQFSLKLX₁₈SVTAX₁₉DTAVYY |
| | | CARMPSFITLASLSTX₂₀EGYFDFWGQGTX₂₁X₂₂TVSS |
| | | X₁ = E or Q |
| | | X₂ = P or A |
| | | X₃ = V or L |
| | | X₄ = S or P |
| | | X₅ = E or G |
| | | X₆ = T or A or S |
| | | X₇ = S or T X₈ = G or S or Y X₉ = S or A |
| | | X₁₀ = I or V |
| | | X₁₁ = Q or K |
| | | X₁₂ = P or F X₁₃ = G or K X₁₄ = L or M |
| | | X₁₅ = I or M X₁₆ = V or I X₁₇ = V or R |
| | | X₁₈ = S or H |
| | | X₁₉ = A or D |
| | | X₂₀ = W, F, Y, I, V, L X₂₁ = M, V, L, A, R, |
| | | N, P, Q, E, G, I, H, K, F, S, T, W or Y |
| | | X₂₂ = V, T or L |
| 14A6 VL | 165 | DIQMTQSPSX₁LSASVGDRVTX₂X₃CKASQSIHKNLAW |
| PARENTAL | | YQQKX₄GX₅X₁₅PKX₆LIYYAX₇X₈LQTGX₉PSRFSGS |
| CONSENSUS | | GSGTDFTLTISX₁₀LQPEDX₁₁ATYX₁₂CQQYYSGX₁₃ |
| | | TFGGGTKVEX₁₄K |
| | | X₁ = L or S |
| | | X₂ = L or I |
| | | X₃ = N or T |
| | | X₄ = L or P |
| | | X₅ = E or K |
| | | X₆ = F or L |
| | | X₇ = N, S, T, G or D |
| | | X₈ = S, N, T, Y or Q |
| | | X₉ = I or V |
| | | X₁₀ = G or S |
| | | X₁₁ = V or F |
| | | X₁₂ = F or Y |
| | | X₁₃ = W, F, Y, I, V or L |
| | | X₁₄ = L or I |
| | | X₁₅ = A or V |
| 14A6 VL HUMANIZED | 166 | DIQMTQSPSSLSASVGDRVTITCKASQSIHKNLAWYQ |
| CONSENSUS | | QKPGKX₆PKX₁LIYYAX₂X₃LQTGX₄PSRFSGSGSGTDF |
| | | TLTISSLQPEDX₇ATYYCQQYYSGX₅TFGGGTKVEIK |
| | | X₁ = L or F |
| | | X₂ = N, S, T, G or D |
| | | X₃ = S, N, T, Y or Q |
| | | X₄ = V or I |
| | | X₅ = W, F, Y, I, V or L |
| | | X₆ = A or V |
| | | X₇ = F or V |
| 31C6 H-CDR2 | 167 | YIDPYNX₁X₂AKYX₃X₄KFX₅G |
| CONSENSUS | | X₁ = D, R, L, K, F, S, Y or V |
| | | X₂ = G, R, N, Q, E, L K, S, Y or V |
| | | X₃ = N, A or S |
| | | X₄ = E or Q |
| | | X₅ = K or Q |
| 31C6 L-CDR2 | 168 | X₁X₂KTLAE |
| CONSENSUS | | X₁ = N, A, V, W, S, T, R, H G, I or V |
| | | X₂ = A, N, I, L, T or V |
| 31C6 VH | 169 | EVQLX₁QSGX₂EX₃X₄KPGX₅SVKX₆SCKASGYTFSSY |
| PARENTAL | | VMHWVX₇QX₈PGQX₉LEWIGYIDPYNX₁₀X₁₁AKYX₁₂X₁₃ |
| CONSENSUS | | KFX₁₄GX₁₅X₁₆TLTX₁₇DX₁₈SX₁₉STX₂₀YMELSX₂₁LX₂₂ |
| | | SX₂₃DX₂₄X₂₅VYYCARGGPYGX₂₆YFDVWGX₂₇GTTVTV |
| | | SS |
| | | X₁ = Q or V |
| | | X₂ = P or A |
| | | X₃ = V or L |
| | | X₄ = V or K |
| | | X₅ = S or A |
| | | X₆ = M or V |
| | | X₇ = K or R |
| | | X₈ = K or A |
| | | X₉ = G or R |
| | | X₁₀ = D, R, L, K, F, S, Y or V |
| | | X₁₁ = G, R, N, Q, E, L K, S, Y or V |
| | | X₁₂ = N, A or S |
| | | X₁₃ = E or Q X₁₄ = K or Q X₁₅ = R or K |
| | | X₁₆ = A or V X₁₇ = S or R |
| | | X₁₈ = K or T |
| | | X₁₉ = S, I, A or T |
| | | X₂₀ = A or V |
| | | X₂₁ = R or S |
| | | X₂₂ = T or R |
| | | X₂₃ = D or E |
| | | X₂₄ = S or T |
| | | X₂₅ = A or V |
| | | X₂₆ = W, A, D, E, F, G, I, K, N, |
| | | Q, R, S, T, V or Y |
| | | X₂₇ = A or Q |
| 31C6 VH | 170 | EVQLVQSGAEVKKPGX₁SVKVSCKASGYTFSSYVM |
| HUMANIZED | | HWVRQAPGQX₂LEWIGYIDPYNX₃X₄AKYX₅X₅KFX₇ |
| CONSENSUS | | GRVTLTX₈DX₉SX₁₀STX₁₁YMELSX₁₂LRSX₁₃DT |
| | | X₁₄VYYCARGGPYGX₁₅YFDVWGQGTTVTVSS |
| | | X₁ = A or S |
| | | X₂ = R or G |
| | | X₃ = D, R, L, K, F, S, Y or V |
| | | X₄ = G, R, N, Q, E, L K, S, Y or V |
| | | X₅ = N, A or S |
| | | X₆ = E or Q |
| | | X₇ = K or Q |
| | | X₈ = R or S |
| | | X₉ = T or K |
| | | X₁₀ = A, T or I |
| | | X₁₁ = A or V |
| | | X₁₂ = S or R |
| | | X₁₃ = E or D |
| | | X₁₄ = A or V |
| | | X₁₅ = W, A, D, E, F, G, I, K, N, |
| | | Q, R, S, T, V or Y |
| 31C6 VL | 171 | DIQMTQSPX₁SLSASVGX₂X₃VTITCRASEHIYSYLSW |
| PARENTAL | | YQQKX₄GKX₅PX₆LLX₇YX₈X₉KTLAEGVPSRFSGSGSG |
| CONSENSUS | | TX₁₀FX₁₁LX₁₂1X₁₃SLQPEDX₁₄X₁₅TYYCQHHFGSPLT |
| | | FGX₁₆GTX₁₇LEX₁₈K |
| | | X₁ = A or S |
| | | X₂ = E or D |
| | | X₃ = T or R |
| | | X₄ = Q or P |
| | | X₅ = S, A or V |
| | | X₆ = Q or K |
| | | X₇ = V or I |
| | | X₈ = N, A, Y, W, S, T, I or V |
| | | X₉ = A, N, I, L, T or V |
| | | X₁₀ = Q or D |
| | | X₁₁ = S or T |
| | | X₁₂ = K or T |
| | | X₁₃ = N or S |
| | | X₁₄ = F or V |
| | | X₁₅ = G or A |
| | | X₁₆ = A or Q |
| | | X₁₇ = T or R |
| | | X₁₈ = L or I |
| 31C6 L-VL | 172 | DIQMTQSPSSLSASVGDRVTITCRASEHIYSYLSWYQ |
| HUMANIZED | | QKPGKX₁PKLLIYX₂X₃KTLAEGVPSRFSGSGSGTX₄F |
| CONSENSUS | | TLTISSLQPEDX₅ATYYCQHHFGSPLTFGQGTRLEIK |
| | | X₁ = A or V |
| | | X₂ = N, A, Y, W, S, T, I or V |
| | | X₃ = A, N, I, L, T or V |
| | | X₄ = D or Q |
| | | X₅ = F or V |
| 31C6 H-CDR3 | 173 | GGPYGXYFDV |
| CONSENSUS | | X₁₅ = W, A, D, E, F, G, I, K, |
| | | N, Q, R, S, T, V or Y |
| 31C6 H-CDR3 | 174 | GGPYGAYFDV |
| VARIANT | | |
| 31C6 H-CDR3 | 175 | GGPYGDYFDV |
| VARIANT | | |
| 31C6 H-CDR3 | 176 | GGPYGEYFDV |
| VARIANT | | |
| 31C6 H-CDR3 | 177 | GGPYGFYFDV |
| VARIANT | | |
| 31C6 H-CDR3 | 178 | GGPYGGYFDV |
| VARIANT | | |
| 31C6 H-CDR3 | 179 | GGPYGIYFDV |
| VARIANT | | |
| 31C6 H-CDR3 | 180 | GGPYGKYFDV |
| VARIANT | | |
| 31C6 H-CDR3 | 181 | GGPYGNYFDV |
| VARIANT | | |
| 31C6 H-CDR3 | 182 | GGPYGQYFDV |
| VARIANT | 183 | GGPYGRYFDV |
| 31C6 H-CDR3 | 184 | GGPYGSYFDV |
| VARIANT | 185 | GGPYGTYFDV |
| 31C6 H-CDR3 | 186 | GGPYGVYFDV |
| VARIANT | 187 | GGPYGYYFDV |
| Vibostolimab, | 294 | DIQMTQSPSSLSASVGDRVTITCRASEHIYSYLSWYQQ |
| light chain | | KPGKVPKLLIYNAKTLAEGVPSRFSGSGSGTDFTLTIS |
| | | SLQPEDVATYYCQHHFGSPLTFGQGTRLEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| Vibostolimab, | 295 | EVQLVQSGAEVKKPGSSVKVSCKASGYTFSSYVMHW |
| heavy chain | | VRQAPGQGLEWIGYIDPYNDGAKYAQKFQGRVTLTS |
| | | DKSTSTAYMELSSLRSEDTAVYYCARGGPYGWYFDV |
| | | WGQGTTVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMIS |
| | | RTPEVTCVVVDVSHEDPEVKFNWYVDGVEVHNAKTK |
| | | PREEQYNSTYRVVSVLTVLHQDWLNGKEYKCKVSNK |
| | | ALPAPIEKTISKAKGQPREPQVYTLPPSRDELTKNQ |
| | | VSLTCLVKGFYPSDIAVEWESNGQPENNYKTTPPVL |
| | | DSDGSFFLYSKLTVDKSRWQQGNVFSCSVMHEALHN |
| | | HYTQKSLSLSPGK |

In some embodiments, an anti-TIGIT antibody or antigen binding fragment thereof comprises three light chain CDRs of CDRL1, CDRL2, and CDRL3 and/or three heavy chain CDRs of CDRH1, CDRH2, and CDRH3.

In one embodiment, the anti-TIGIT antibody or antigen binding fragment thereof comprises a CDRH1 having the amino acid sequence as set forth in SEQ ID NO:35, a CDRH2 having the amino acid sequence as set forth in SEQ ID NO:36, a CDRH3 having any of the amino acid sequences as set forth in SEQ ID NOs:37, 103, 104, 105, 106, 107, or 160, a CDRL1 having the amino acid sequence as set forth in SEQ ID NO:38, a CDRL2 having any of the amino acid sequences as set forth in SEQ ID NOs:39, 89, 90, 91, 92, 93, 94, 95, 96, 97, or 161, and a CDRL3 having any of the amino acid sequences as set forth in SEQ ID NOs:40, 98, 99, 100, 101, 102, or 162.

In another embodiment, the anti-TIGIT antibody or antigen binding fragment thereof comprises a CDRH1 having the amino acid sequence as set forth in SEQ ID NO:81, a CDRH2 having the amino acid sequence as set forth in SEQ ID NO:82, a CDRH3 having the amino acid sequence as set forth in SEQ ID NO:83, a CDRL1 having the amino acid sequence as set forth in SEQ ID NO:84, a CDRL2 having the amino acid sequence as set forth in SEQ ID NO:85, and a CDRL3 having the amino acid sequence as set forth in SEQ ID NO:86.

In another embodiment, the anti-TIGIT antibody or antigen binding fragment thereof comprises a CDRH1 having the amino acid sequence as set forth in SEQ ID NO:108, a CDRH2 having any of the amino acid sequences as set forth in SEQ ID NOs:109, 116, 117, 118, 119, 120, 121, 122, 123, 124, 125, 126, 127, 128, 129, 130, 131, 154, 155 or 167, a CDRH3 having any of the amino acid sequences as set forth in SEQ ID NOs:110, 173, 174, 175, 176, 177, 178, 179, 180, 181, 182, 183, 184, 185, 186 or 187, a CDRL1 having the amino acid sequence as set forth in SEQ ID NO:111, a CDRL2 having any of the amino acid sequences as set forth in SEQ ID NOs:112, 132, 133, 134, 135, 136, 137, 138, 139, 140, 141, 142 or 168, and a CDRL3 having the amino acid sequence as set forth in SEQ ID NO:113.

In one embodiment, the anti-TIGIT antibody or antigen binding fragment thereof comprises a CDRH1 having the amino acid sequence as set forth in SEQ ID NO:35, a CDRH2 having the amino acid sequence as set forth in SEQ ID NO:36, a CDRH3 having the amino acid sequence as set forth in SEQ ID NO:37, a CDRL1 having the amino acid sequence as set forth in SEQ ID NO:38, a CDRL2 having the amino acid sequence as set forth in SEQ ID NO:39, and a CDRL3 having the amino acid sequence as set forth in SEQ ID NO:40.

In one embodiment, the anti-TIGIT antibody or antigen binding fragment thereof comprises CDRH1 having the amino acid sequence as set forth in SEQ ID NO:108, a CDRH2 having any one of the amino acid sequences as set forth in SEQ ID NO:109, 154 or 145, a CDRH3 having the amino acid sequence as set forth in SEQ ID NO:110, a CDRL1 having the amino acid sequence as set forth in SEQ ID NO:111, a CDRL2 having the amino acid sequence as set forth in SEQ ID NO:112, and a CDRL3 having the amino acid sequence as set forth in SEQ ID NO:113.

In another embodiment, the anti-TIGIT antibody or antigen binding fragment thereof comprises a CDRH1 having the amino acid sequence as set forth in SEQ ID NO:108, a CDRH2 having the amino acid sequence as set forth in SEQ ID NO: 154, a CDRH3 having the amino acid sequence as set forth in SEQ ID NO:110, a CDRL1 having the amino acid sequence as set forth in SEQ ID NO:111, a CDRL2 having the amino acid sequence as set forth in SEQ ID NO:112, and a CDRL3 having the amino acid sequence as set forth in SEQ ID NO:113.

In some embodiments, the anti-TIGIT antibody or antigen binding fragment thereof comprises a light chain comprising or consisting of the amino acid sequence as set forth in SEQ ID NO:294 and a heavy chain comprising or consisting of the amino acid sequence as set forth in SEQ ID NO:295.

In one embodiment, the anti-TIGIT antibody or antigen binding fragment thereof comprises a variable heavy chain region and a variable light chain variable region. In one embodiment, the anti-TIGIT antibody or antigen binding fragment thereof comprises a variable heavy chain region comprising the amino acid sequence as set forth in SEQ ID NO:41 and a variable light chain region comprising the amino acid sequence as set forth in SEQ ID NO:42.

In one embodiment, the anti-TIGIT antibody or antigen binding fragment thereof comprises a variable heavy chain region comprising the amino acid sequence as set forth in SEQ ID NO:87 and a variable light chain region comprising the amino acid sequence as set forth in SEQ ID NO:88.

In one embodiment, the anti-TIGIT antibody or antigen binding fragment thereof comprises a variable heavy chain region comprising the amino acid sequence as set forth in SEQ ID NO: 114 and a variable light chain region comprising the amino acid sequence as set forth in SEQ ID NO:115.

In one embodiment, the anti-TIGIT antibody or antigen binding fragment thereof comprises a variable heavy chain region comprising any of the amino acid sequences as set forth in SEQ ID NOs: 43-58, 65-75 and 87 and a variable light chain region comprising any one of the amino acid sequences as set forth in SEQ ID NOs: 59-64, 76-80 and 88.

In one embodiment, the anti-TIGIT antibody or antigen binding fragment thereof comprises a variable heavy chain region comprising any of the amino acid sequences as set forth in SEQ ID NOs: 144-149 and a variable light chain region comprising any of the amino acid sequences as set forth in SEQ ID NOs: 150-153.

In one embodiment, the anti-TIGIT antibody or antigen binding fragment thereof comprises a variable heavy chain region comprising a variable heavy chain region comprising the amino acid sequence as set forth in SEQ ID NO:148 and a variable light chain region comprising the amino acid sequence as set forth in SEQ ID NO: 152.

In one embodiment, the anti-TIGIT antibody or antigen binding fragment thereof comprises a variable heavy chain region comprising the amino acid sequence as set forth in SEQ ID NO:147 and a variable light chain region comprising the amino acid sequence as set forth in SEQ ID NO:150.

In one embodiment, the anti-TIGIT antibody or antigen binding fragment thereof comprises a variable heavy chain region comprising the amino acid sequence as set forth in SEQ ID NO:148 and a variable light chain region comprising the amino acid sequence as set forth in SEQ ID NO:153.

In one embodiment, the anti-TIGIT antibody or antigen binding fragment thereof comprises a variable heavy chain region comprising the amino acid sequence as set forth in SEQ ID NO:163 and a variable light chain region comprising the amino acid sequence as set forth in SEQ ID NO:165.

In one embodiment, the anti-TIGIT antibody or antigen binding fragment thereof comprises a variable heavy chain region comprising the amino acid sequence as set forth in SEQ ID NO:169 and a variable light chain region comprising the amino acid sequence as set forth in SEQ ID NO:171.

In one embodiment, the anti-TIGIT antibody or antigen binding fragment thereof comprises a variable heavy chain region comprising the amino acid sequence as set forth in SEQ ID NO:164 and a variable light chain region comprising the amino acid sequence as set forth in SEQ ID NO:166.

In one embodiment, the anti-TIGIT antibody or antigen binding fragment thereof comprises a variable heavy chain region comprising the amino acid sequence as set forth in SEQ ID NO:170 and a variable light chain region comprising the amino acid sequence as set forth in SEQ ID NO:172.

TABLE 7
Exemplary sequences of anti-TIGIT antibodies

| | SEQ ID | |
|---|---|---|
| Description | NO | SEQUENCE |
| 14D7 H-CDR1 | 188 | GAWMD |
| 14D7 H-CDR2 | 189 | EIRTKVNNHATNYGESVKG |
| 14D7 H-CDR3 | 190 | ALYDGFYFDY |
| 14D7 L-CDR1 | 191 | SASSSVSSGYLY |
| 14D7 L-CDR2 | 192 | GTSTLAS |
| 14D7 L-CDR3 | 193 | HQWSSFPYT |
| 14D7 VH | 194 | EVKLEESGGGLVQPGGSMKLSCVASGFTFSGAWMDWVRQSPEKGLEWVA |
| PARENTAL | | EIRTKVNNHATNYGESVKGRFTISRDDSKSSVYLQMNNLRAEDSGIYYC |
| | | RGALYDGFYFDYWGQGTTLTVSS |
| 14D7 VL | 195 | QIVLTQSPAIMSASPGEKVNLTCSASSSVSSGYLYWYQQKPGSSPKLWI |
| PARENTAL | | YGTSTLASGVPARFSGSGSGTSYSLTISNMEAEDAASYFCHQWSSFPYT |
| | | FGGGTKLEMK |
| Hu14D7 VH | 196 | EVQLVESGGGLVQPGGSLKLSCAASGFTFSGAX₁X₂DWVRQAPGKGLEW |
| humanized | | VAEIRTKVNNHATNYGESVKGRFTISRDX₃SKX₄X₅VYLQX₆X₇X₈LRAE |
| consensus | | DX₉AVYYCRGALYX₁₀X₁₁FYFDYWGQGTLVTVSS |
| sequence | | X₁ = W, A, R, N, D, Q, E, G, H, I, L, K, F, P, S, T, Y, V |
| | | X₂ = M, V, L, I, G, A, S, T |
| | | X₃ = D, A, R, N, Q, E, G, H, I, L, K, F, S, T, Y, V |
| | | X₄ = S, N |
| | | X₅ = T, S |
| | | X₆ = M, L |
| | | X₇ = N, S |
| | | X₈ = S, N |
| | | X₉ = T, S |
| | | X₁₀ = D, A, R, N, Q, E, G, H, I, L, K, F, P, S, T, W, Y, V |
| | | X₁₁ = G, A, R, N, D, Q, E, H, I, L, K, F, P, S, T, W, Y, V |
| Hu14D7 VH1 | 197 | EVQLVESGGGLVQPGGSLKLSCAASGFTFSGAWMDWVRQAPGKGLEWVA |
| (Humanized VH | | EIRTKVNNHATNYGESVKGRFTISRDDSKSTVYLQMNSLRAEDTAVYYC |
| chain) | | RGALYDGFYFDYWGQGTLVTVSS |
| Hu14D7 VH2 | 198 | EVQLVESGGGLVQPGGSLKLSCAASGFTFSGAWMDWVRQAPGKGLEWVA |
| (Humanized VH | | EIRTKVNNHATNYGESVKGRFTISRDDSKSSVYLQMNSLRAEDTAVYYC |
| chain) | | RGALYDGFYFDYWGQGTLVTVSS |
| Hu14D7 VH3 | 199 | EVQLVESGGGLVQPGGSLKLSCAASGFTFSGAWMDWVRQAPGKGLEWVA |
| (Humanized VH | | EIRTKVNNHATNYGESVKGRFTISRDDSKNTVYLQMNSLRAEDTAVYYC |
| chain) | | RGALYDGFYFDYWGQGTLVTVSS |
| Hu14D7 VL | 200 | EIVLTQSPATLSLSPGERAX₁ILSCSASSSVSSGYLYWYQQKPGQAPX₇LX₂ |
| humanized | | IYGTSTLASGX₈PARFSGSGSGTDYTLTISSX₃EPEDX₄AVYYCHQX₅SSFP |
| consensus | | YTFGQGTKLEX₆K |
| Sequence | | X₁ = T, S |
| | | X₂ = W, A, R, N, D, Q, E, G, H, I, L, K, F, P, S, T, Y, V |
| | | X₃ = L, V, I |
| | | X₄ = F, V, L, I, T |
| | | X₅ = W, A, R, N, D, Q, E, G, H, I, L, K, F, P, S, T, Y, V |
| | | X₆ = I, L |
| | | X₇ = K, R |
| | | X₈ = V, I |
| Hu14D7V L1 | 201 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQWSSFPYTFGQGTKL |
| chain) | | EIK |
| Hu14D7V L2 | 202 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQWSSFPYTFGQGTKL |
| chain) | | EIK |
| Hu14D7V L3 | 203 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQWSSFPYTFGQGTKLE |
| chain) | | IK |
| 26B10 H-CDR1 | 204 | EFTMH |
| 26B10 H-CDR2 | 205 | GLKPDNGGISYNQKFKG |
| 26B10 H-CDR3 | 206 | GAYYRYDADY |
| 26B10 L-CDR1 | 207 | KASQDVKTAVA |
| 26B10 L-CDR2 | 208 | SASYRNT |
| 26B10 L-CDR3 | 209 | QQHYSTPFT |
| 26B10 VH | 210 | EVQLQQSGPELVKPGASVKISCKTSGYTFTEFTMHWVKQSHGKSLEWIGGL |
| PARENTAL | | KPDNGGISYNQKFKGRATLAVDKSSNTAYMELRSLTSEDSAVYYCARGAY |
| | | YRYDADYWGQGTTLTVSS |
| 26B10 VL | 211 | DIVLTQSHKFMSTSVGDRVSITCKASQDVKTAVAWYQQKSGQSPKLLIYSA |
| PARENTAL | | SYRNTGVPDRFTGSGSGTDFTFTIDSVQAEDLAVYFCQQHYSTPFTFGTGTK |
| | | LELK |
| 26B10 VH | 212 | EVQLVQSGAEVKKPGASVKISCKX₁SGYTFTEFTX₂HWVX₃QAPGKGLEWIG |
| HUMANIZED | | GLKPDX₄X₅GISYNQKFKGRATLTVDX₆STX₇TAYX₈ELSSLRSEDX₉AVYYCA |
| CONSESUS | | RGAYYRYX₁₀X₁₁DYWGQGTLVTVSS |
| Sequence | | X₁ = T, V |
| | | X₂ = M, V, L, I, G, A, S, T |
| | | X₃ = K, R |
| | | X₄ = N, A, R, D, Q, E, G, H, I, L, K, F, P, S, T, W, Y, V |
| | | X₅ = G, A, R, N, D, Q, E, H, I, L, K, F, P, S, T, W, Y, V |
| | | X₆ = k, t, d, s |
| | | X₇ = N, S |
| | | X₈ = M, V, L, I, G, A, S, T |
| | | X₉ = T, S |
| | | X₁₀ = D, A, R, N, Q, E, G, H, I, L, K, F, P, S, T, W, Y, V |
| | | X₁₁ = A, R, N, D, Q, E, G, H, I, L, K, F, P, S, T, W, Y, V, M |
| Hu26B10 VH1 | 213 | EVQLVQSGAEVKKPGASVKISCKTSGYTFTEFTMHWVKQAPGKGLEWIGG |
| (Humanized VH | | LKPDNGGISYNQKFKGRATLTVDKSTNTAYMELSSLRSEDTAVYYCARGAY |
| chain) | | YRYDADYWGQGTLVTVSS |
| Hu26B10 VH2 | 214 | EVQLVQSGAEVKKPGASVKISCKTSGYTFTEFTMHWVRQAPGKGLEWIGGL |
| (Humanized VH | | KPDNGGISYNQKFKGRATLTVDKSTSTAYMELSSLRSEDTAVYYCARGAYY |
| chain) | | RYDADYWGQGTLVTVSS |
| Hu26B10 VH3 | 215 | EVQLVQSGAEVKKPGASVKISCKVSGYTFTEFTMHWVRQAPGKGLEWIGG |
| (Humanized VH | | LKPDNGGISYNQKFKGRATLTVDTSTSTAYMELSSLRSEDTAVYYCARGAY |
| chain) | | YRYDADYWGQGTLVTVSS |
| 26B10 VL | 216 | DIQLTQSPSSLSASVGDRVTITCKASQDVKTAVAWYQQKPGKAPKLLIYSAS |
| HUMANIZED | | YRX₁X₂GVPX₃RFSGSGSGTDFTX₄TISSLQPEDFATYYCQQHYSTPFTFGQG |
| CONSESUS | | TKLEIK |
| sequence | | X₁ = N, Q, D, E |
| | | X₂ = T, S, A |
| | | X₃ = D, S |
| | | X₄ = F, L |
| Hu26B10 VL1 | 217 | DIQLTQSPSSLSASVGDRVTITCKASQDVKTAVAWYQQKPGKAPKLLIYSAS |
| (Humanized VL | | YRNTGVPDRFSGSGSGTDFTFTISSLQPEDFATYYCQQHYSTPFTFGQGTKL |
| chain) | | EIK |
| Hu26B10 VL2 | 218 | DIQLTQSPSSLSASVGDRVTITCKASQDVKTAVAWYQQKPGKAPKLLIYSAS |
| (Humanized VL | | YRNTGVPSRFSGSGSGTDFTFTISSLQPEDFATYYCQQHYSTPFTFGQGTKL |
| chain) | | EIK |
| Hu26B10 VL3 | 219 | DIQLTQSPSSLSASVGDRVTITCKASQDVKTAVAWYQQKPGKAPKLLIYSAS |
| (Humanized VL | | YRNTGVPSRFSGSGSGTDFTLTISSLQPEDFATYYCQQHYSTPFTFGQGTKL |
| chain) | | EIK |
| 14D7 H-CDR3 | 220 | ALYEGFYFDY |
| (D104E) | | |
| 14D7 H-CDR3 | 221 | ALYDAFYFDY |
| (G105A) | | |
| 14D7 H-CDR3 | 222 | ALYDSFYFDY |
| (G105S) | | |
| Hu14D7 VH1 | 223 | EVQLVESGGGLVQPGGSLKLSCAASGFTFSGAWMDWVRQAPGKGLEWVAE |
| (Humanized VH | | IRTKVNNHATNYGESVKGRFTISRDDSKSTVYLQMNSLRAEDTAVYYCRG |
| chain) (D104E) | | ALYEGFYFDYWGQGTLVTVSS |
| Hu14D7 VH1 | 224 | EVQLVESGGGLVQPGGSLKLSCAASGFTFSGAWMDWVRQAPGKGLEWVAE |
| (Humanized VH | | IRTKVNNHATNYGESVKGRFTISRDDSKSTVYLQMNSLRAEDTAVYYCRG |
| chain) (G105A) | | ALYDAFYFDYWGQGTLVTVSS |
| Hu14D7 VH1 | 225 | EVQLVESGGGLVQPGGSLKLSCAASGFTFSGAWMDWVRQAPGKGLEWVAE |
| (Humanized VH | | IRTKVNNHATNYGESVKGRFTISRDDSKSTVYLQMNSLRAEDTAVYYCRG |
| chain) (G105S) | | ALYDSFYFDYWGQGTLVTVSS |
| Hu14D7 VH2 | 226 | EVQLVESGGGLVQPGGSLKLSCAASGFTFSGAWMDWVRQAPGKGLEWVAE |
| (Humanized VH | | IRTKVNNHATNYGESVKGRFTISRDDSKSSVYLQMNSLRAEDTAVYYCRG |
| chain) (D104E) | | ALYDEFYFDYWGQGTLVTVSS |
| Hu14D7 VH2 | 227 | EVQLVESGGGLVQPGGSLKLSCAASGFTFSGAWMDWVRQAPGKGLEWVAE |
| (Humanized VH | | IRTKVNNHATNYGESVKGRFTISRDDSKSSVYLQMNSLRAEDTAVYYCRG |
| chain) (G105A) | | ALYDAFYFDYWGQGTLVTVSS |
| Hu14D7 VH2 | 228 | EVQLVESGGGLVQPGGSLKLSCAASGFTFSGAWMDWVRQAPGKGLEWVAE |
| (Humanized VH | | IRTKVNNHATNYGESVKGRFTISRDDSKSSVYLQMNSLRAEDTAVYYCRG |
| chain) (G105S) | | ALYDSFYFDYWGQGTLVTVSS |
| Hu14D7 VH3 | 229 | EVQLVESGGGLVQPGGSLKLSCAASGFTFSGAWMDWVRQAPGKGLEWVAE |
| (Humanized VH | | IRTKVNNHATNYGESVKGRFTISRDDSKNTVYLQMNSLRAEDTAVYYCRG |
| chain) (D104E) | | ALYEGFYFDYWGQGTLVTVSS |
| Hu14D7 VH3 | 230 | EVQLVESGGGLVQPGGSLKLSCAASGFTFSGAWMDWVRQAPGKGLEWVAE |
| (Humanized VH | | IRTKVNNHATNYGESVKGRFTISRDDSKNTVYLQMNSLRAEDTAVYYCRG |
| chain) (G105A) | | ALYDAFYFDYWGQGTLVTVSS |
| Hu14D7 VH3 | 231 | EVQLVESGGGLVQPGGSLKLSCAASGFTFSGAWMDWVRQAPGKGLEWVAE |
| (Humanized VH | | IRTKVNNHATNYGESVKGRFTISRDDSKNTVYLQMNSLRAEDTAVYYCRG |
| chain) (G105S) | | ALYDSFYFDYWGQGTLVTVSS |
| 14D7 L-CDR3 | 232 | HQASSFPYT |
| (W92A) | | |
| 14D7 L- | 233 | HQDSSFPYT |
| CDR3(W92D) | | |
| 14D7 L- | 234 | HQESSFPYT |
| CDR3(W92E) | | |
| 14D7 L- | 235 | HQFSSFPYT |
| CDR3(W92F) | | |
| 14D7 L- | 236 | HQGSSFPYT |
| CDR3(W92G) | | |
| 14D7 L-CDR3 | 237 | HQHSSFPYT |
| (W92H) | | |
| Hu14D7VL1 | 238 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQASSFPYTFGQGTKLE |
| chain) (W92A) | | IK |
| Hu14D7VL1 | 239 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQDSSFPYTFGQGTKLE |
| chain) (W92D) | | IK |
| Hu14D7VL1 | 240 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQESSFPYTFGQGTKLE |
| chain) (W92E) | | IK |
| Hu14D7VL1 | 241 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQFSSFPYTFGQGTKLE |
| chain) (W92F) | | IK |
| Hu14D7VL1 | 242 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQGSSFPYTFGQGTKLE |
| chain) (W92G) | | IK |
| Hu14D7VL1 | 243 | EIVLTQSPATLSLSPGERATLSCSASSSVSSGYLYWYQQKPGQAPKLWIYGTS |
| (Humanized VL | | TLASGVPARFSGSGSGTDYTLTISSLEPEDFAVYYCHQHSSFPYTFGQGTKLE |
| chain) (W92H) | | IK |
| Hu14D7VL2 | 244 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQASSFPYTFGQGTKLE |
| chain) (W92A) | | IK |
| Hu14D7VL2 | 245 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQDSSFPYTFGQGTKLE |
| chain) (W92D) | | IK |
| Hu14D7VL2 | 246 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQESSFPYTFGQGTKLE |
| chain) (W92E) | | IK |
| Hu14D7VL2 | 247 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQFSSFPYTFGQGTKLE |
| chain) (W92F) | | IK |
| Hu14D7VL2 | 248 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQGSSFPYTFGQGTKLE |
| chain) (W92G) | | IK |
| Hu14D7VL2 | 249 | EIVLTQSPATLSLSPGERATLSCSASSSVSSGYLYWYQQKPGQAPRLWIYGTS |
| (Humanized VL | | TLASGVPARFSGSGSGTDYTLTISSLEPEDFAVYYCHQHSSFPYTFGQGTKLE |
| chain) (W92H) | | IK |
| Hu14D7VL3 | 250 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQASSFPYTFGQGTKLE |
| chain) (W92A) | | IK |
| Hu14D7VL3 | 251 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQDSSFPYTFGQGTKLE |
| chain) (W92D) | | IK |
| Hu14D7VL3 | 252 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQESSFPYTFGQGTKLE |
| chain) (W92E) | | IK |
| Hu14D7V L3 | 253 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQFSSFPYTFGQGTKLE |
| chain) (W92H) | | IK |
| Hu14D7V L3 | 254 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQGSSFPYTFGQGTKLE |
| chain) (W92G) | | IK |
| Hu14D7VL3 | 255 | EIVLTQSPATLSLSPGERATLSCSASSSVSSGYLYWYQQKPGQAPRLWIYGTS |
| (Humanized VL | | TLASGIPARFSGSGSGTDYTLTISSLEPEDFAVYYCHQHSSFPYTFGQGTKLE |
| chain) (W92H) | | IK |
| 26B10 H-CDR2 | 256 | GLKPDQGGISYNQKFKG |
| (N55Q) | | |
| 26B10 H- | 257 | GLKPDDGGISYNQKFKG |
| CDR2(N55D) | | |
| 26B10 H- | 258 | GLKPDNAGISYNQKFKG |
| CDR2(N56A) | | |
| 26B10 H- | 259 | GLKPDTGGISYNQKFKG |
| CDR2(N55T) | | |
| 26B10 H- | 260 | GLKPDSGGISYNQKFKG |
| CDR2(N55S) | | |
| 26B10 H- | 261 | GLKPDGGGISYNQKFKG |
| CDR2(N55G) | | |
| 26B10 H-CDR2 | 262 | GLKPDNSGISYNQKFKG |
| (G56S) | | |
| 26B10 H- | 263 | GLKPDNTGISYNQKFKG |
| CDR2(G56T) | | |
| Hu26B10 VH1 | 264 | EVQLVQSGAEVKKPGASVKISCKTSGYTFTEFTMHWVKQAPGKGLEWIGG |
| (Humanized VH | | LKPDQGGISYNQKFKGRATLTVDKSTNTAYMELSSLRSEDTAVYYCARGA |
| chain)(N55Q) | | YYRYDADYWGQGTLVTVSS |
| Hu26B10 VH1 | 265 | EVQLVQSGAEVKKPGASVKISCKTSGYTFTEFTMHWVKQAPGKGLEWIGG |
| (Humanized VH | | LKPDDGGISYNQKFKGRATLTVDKSTNTAYMELSSLRSEDTAVYYCARGAY |
| chain)(N55D) | | YRYDADYWGQGTLVTVSS |
| Hu26B10 VH1 | 266 | EVQLVQSGAEVKKPGASVKISCKTSGYTFTEFTMHWVKQAPGKGLEWIGG |
| (Humanized VH | | LKPDNAGISYNQKFKGRATLTVDKSTNTAYMELSSLRSEDTAVYYCARGAY |
| chain)(G56A) | | YRYDADYWGQGTLVTVSS |
| Hu26B10 VH1 | 267 | EVQLVQSGAEVKKPGASVKISCKTSGYTFTEFTMHWVKQAPGKGLEWIGG |
| (Humanized VH | | LKPDTGGISYNQKFKGRATLTVDKSTNTAYMELSSLRSEDTAVYYCARGAY |
| chain)(N55T) | | YRYDADYWGQGTLVTVSS |
| Hu26B10 VH1 | 268 | EVQLVQSGAEVKKPGASVKISCKTSGYTFTEFTMHWVKQAPGKGLEWIGG |
| (Humanized VH | | LKPDSGGISYNQKFKGRATLTVDKSTNTAYMELSSLRSEDTAVYYCARGAY |
| chain)(N55S) | | YRYDADYWGQGTLVTVSS |
| Hu26B10 VH1 | 269 | EVQLVQSGAEVKKPGASVKISCKTSGYTFTEFTMHWVKQAPGKGLEWIGG |
| (Humanized VH | | LKPDGGGISYNQKFKGRATLTVDKSTNTAYMELSSLRSEDTAVYYCARGA |
| chain)(N55G) | | YYRYDADYWGQGTLVTVSS |
| Hu26B10 VH1 | 270 | EVQLVQSGAEVKKPGASVKISCKTSGYTFTEFTMHWVKQAPGKGLEWIGG |
| (Humanized VH | | LKPDNSGISYNQKFKGRATLTVDKSTNTAYMELSSLRSEDTAVYYCARGAY |
| chain)(G56S) | | YRYDADYWGQGTLVTVSS |
| Hu26B10 VH1 | 271 | EVQLVQSGAEVKKPGASVKISCKTSGYTFTEFTMHWVKQAPGKGLEWIGG |
| (Humanized VH | | LKPDNTGISYNQKFKGRATLTVDKSTNTAYMELSSLRSEDTAVYYCARGAY |
| chain)(G56T) | | YRYDADYWGQGTLVTVSS |
| Hu26B10 VH2 | 272 | EVQLVQSGAEVKKPGASVKISCKTSGYTFTEFTMHWVRQAPGKGLEWIGGL |
| (Humanized VH | | KPDQGGISYNQKFKGRATLTVDKSTSTAYMELSSLRSEDTAVYYCARGAYY |
| chain)(N55Q) | | RYDADYWGQGTLVTVSS |
| Hu26B10 VH2 | 273 | EVQLVQSGAEVKKPGASVKISCKTSGYTFTEFTMHWVRQAPGKGLEWIGGL |
| (Humanized VH | | KPDDGGISYNQKFKGRATLTVDKSTSTAYMELSSLRSEDTAVYYCARGAYY |
| chain)(N55D) | | RYDADYWGQGTLVTVSS |
| Hu26B10 VH1 | 274 | EVQLVQSGAEVKKPGASVKISCKTSGYTFTEFTMHWVRQAPGKGLEWIGGL |
| (Humanized VH | | KPDNAGISYNQKFKGRATLTVDKSTSTAYMELSSLRSEDTAVYYCARGAYY |
| chain)(G56A) | | RYDADYWGQGTLVTVSS |
| Hu26B10 VH2 | 275 | EVQLVQSGAEVKKPGASVKISCKTSGYTFTEFTMHWVRQAPGKGLEWIGGL |
| (Humanized VH | | KPDTGGISYNQKFKGRATLTVDKSTSTAYMELSSLRSEDTAVYYCARGAYY |
| chain)(N55T) | | RYDADYWGQGTLVTVSS |
| Hu26B10 VH2 | 276 | EVQLVQSGAEVKKPGASVKISCKTSGYTFTEFTMHWVRQAPGKGLEWIGGL |
| (Humanized VH | | KPDSGGISYNQKFKGRATLTVDKSTSTAYMELSSLRSEDTAVYYCARGAYY |
| chain)(N55S) | | RYDADYWGQGTLVTVSS |
| Hu26B10 VH2 | 277 | EVQLVQSGAEVKKPGASVKISCKTSGYTFTEFTMHWVRQAPGKGLEWIGGL |
| (Humanized VH | | KPDGGGISYNQKFKGRATLTVDKSTSTAYMELSSLRSEDTAVYYCARGAYY |
| chain)(N55G) | | RYDADYWGQGTLVTVSS |
| Hu26B10 VH2 | 278 | EVQLVQSGAEVKKPGASVKISCKTSGYTFTEFTMHWVRQAPGKGLEWIGGL |
| (Humanized VH | | KPDNSGISYNQKFKGRATLTVDKSTSTAYMELSSLRSEDTAVYYCARGAYY |
| chain)(G56S) | | RYDADYWGQGTLVTVSS |
| Hu26B10 VH2 | 279 | EVQLVQSGAEVKKPGASVKISCKTSGYTFTEFTMHWVRQAPGKGLEWIGGL |
| (Humanized VH | | KPDNTGISYNQKFKGRATLTVDKSTSTAYMELSSLRSEDTAVYYCARGAYY |
| chain)(G56T) | | RYDADYWGQGTLVTVSS |
| Hu26B10 VH3 | 280 | EVQLVQSGAEVKKPGASVKISCKVSGYTFTEFTMHWVRQAPGKGLEWIGG |
| (Humanized VH | | LKPDQGGISYNQKFKGRATLTVDTSTSTAYMELSSLRSEDTAVYYCARGAY |
| chain) (N55Q) | | YRYDADYWGQGTLVTVSS |
| Hu26B10 VH3 | 281 | EVQLVQSGAEVKKPGASVKISCKVSGYTFTEFTMHWVRQAPGKGLEWIGG |
| (Humanized VH | | LKPDDGGISYNQKFKGRATLTVDTSTSTAYMELSSLRSEDTAVYYCARGAY |
| chain)(N55D) | | YRYDADYWGQGTLVTVSS |
| Hu26B10 VH3 | 282 | EVQLVQSGAEVKKPGASVKISCKVSGYTFTEFTMHWVRQAPGKGLEWIGG |
| (Humanized VH | | LKPDNAGISYNQKFKGRATLTVDTSTSTAYMELSSLRSEDTAVYYCARGAY |
| chain)(G56A) | | YRYDADYWGQGTLVTVSS |
| Hu26B10 VH3 | 283 | EVQLVQSGAEVKKPGASVKISCKVSGYTFTEFTMHWVRQAPGKGLEWIGG |
| (Humanized VH | | LKPDTGGISYNQKFKGRATLTVDTSTSTAYMELSSLRSEDTAVYYCARGAY |
| chain)(N55T) | | YRYDADYWGQGTLVTVSS |
| Hu26B10 VH3 | 284 | EVQLVQSGAEVKKPGASVKISCKVSGYTFTEFTMHWVRQAPGKGLEWIGG |
| (Humanized VH | | LKPDSGGISYNQKFKGRATLTVDTSTSTAYMELSSLRSEDTAVYYCARGAY |
| chain)(N55S) | | YRYDADYWGQGTLVTVSS |
| Hu26B10 VH3 | 285 | EVQLVQSGAEVKKPGASVKISCKVSGYTFTEFTMHWVRQAPGKGLEWIGG |
| (Humanized VH | | LKPDGGGISYNQKFKGRATLTVDTSTSTAYMELSSLRSEDTAVYYCARGAY |
| chain)(N55G) | | YRYDADYWGQGTLVTVSS |
| Hu26B10 VH3 | 286 | EVQLVQSGAEVKKPGASVKISCKVSGYTFTEFTMHWVRQAPGKGLEWIGG |
| (Humanized VH | | LKPDNSGISYNQKFKGRATLTVDTSTSTAYMELSSLRSEDTAVYYCARGAY |
| chain)(G56S) | | YRYDADYWGQGTLVTVSS |
| Hu26B10 VH3 | 287 | EVQLVQSGAEVKKPGASVKISCKVSGYTFTEFTMHWVRQAPGKGLEWIGG |
| (Humanized VH | | LKPDNTGISYNQKFKGRATLTVDTSTSTAYMELSSLRSEDTAVYYCARGAY |
| chain)(G56T) | | YRYDADYWGQGTLVTVSS |
| hTIGIT epitope | 288 | SSTTAQVNWEQQDQL |
| (24-41) | | |
| hTIGIT epitope | 289 | IYHTYPDGT |
| (85-93) | | |
| hTIGIT epitope | 290 | GRIFL |
| (96-100) | | |

In one embodiment, the anti-TIGIT antibody or antigen binding fragment comprises a CDRH1 having the amino acid sequence as set forth in SEQ ID NO:188, a CDRH2 having the amino acid sequence as set forth in SEQ ID NO:189, a CDRH3 having any of the amino acid sequences as set forth in SEQ ID NOs:190, 220, 221, or 222, a CDRL1 having the amino acid sequence as set forth in SEQ ID NO:191, a CDRL2 having the amino acid sequence as set forth in SEQ ID NO:192, and a CDRL3 having any of the amino acid sequences as set forth in SEQ ID NOs:193, 232, 233, 234, 235, 236, or 237.

In one embodiment, the anti-TIGIT antibody or antigen binding fragment thereof comprises a CDRH1 having the amino acid sequence as set forth in SEQ ID NO:204, a CDRH2 having any of the amino acid sequences as set forth in SEQ ID NOs: 205, 256, 257, 258, 259, 260, 261, 262, or 263, a CDRH3 having the amino acid sequence as set forth in SEQ ID NO:206, a CDRL1 having the amino acid sequence as set forth in SEQ ID NO:207, a CDRL2 having the amino acid sequence as set forth in SEQ ID NO:208, and a CDRL3 having the amino acid sequence as set forth in SEQ ID NO:209.

In one embodiment, the anti-TIGIT antibody or antigen binding fragment thereof comprises a variable heavy chain region and a variable light chain variable region. In one embodiment, the anti-TIGIT antibody or antigen binding fragment thereof comprises a variable heavy chain region comprising the amino acid sequence as set forth in SEQ ID NO:194 and a variable light chain region comprising the amino acid sequence as set forth in SEQ ID NO:195.

In one embodiment, the anti-TIGIT antibody or antigen binding fragment thereof comprises a variable heavy chain region comprising the amino acid sequence as set forth in SEQ ID NO: 196 and a variable light chain region comprising the amino acid sequence as set forth in SEQ ID NO:200.

In one embodiment, the anti-TIGIT antibody or antigen binding fragment thereof comprises a variable heavy chain region comprising the amino acid sequence as set forth in SEQ ID NO: 210 and a variable light chain region comprising the amino acid sequence as set forth in SEQ ID NO:211.

In one embodiment, the anti-TIGIT antibody or antigen binding fragment thereof comprises a variable heavy chain region comprising the amino acid sequence as set forth in SEQ ID NO: 212 and a variable light chain region comprising the amino acid sequence as set forth in SEQ ID NO:216.

In one embodiment, the anti-TIGIT antibody or antigen binding fragment thereof comprises a variable heavy chain region comprising any of the amino acid sequences as set forth in SEQ ID NOs: 197, 198, 199, 223, 224, 225, 226, 227, 228, 229, 230, and 231 and a variable light chain region comprising any of the amino acid sequences as set forth in SEQ ID NOs: 201, 202, 203, 238, 239, 240, 241, 242, 243, 244, 245, 246, 247, 248, 249, 250, 251, 252, 253, 254, and 255.

In one embodiment, the anti-TIGIT antibody or antigen binding fragment thereof comprises a variable heavy chain region comprising any of the amino acid sequences as set forth in SEQ ID NOs: 213, 214, 215, 264, 265, 266, 267, 268, 269, 270, 271, 272, 273, 274, 275, 276, 277, 278, 279, 280, 281, 282, 283, 284, 285, and 286 and a variable light chain region comprising any of the amino acid sequences as set forth in SEQ ID NOs: 217, 218, and 219.

Additional anti-TIGIT antibodies which may be used in the formulations described herein include those disclosed, for example, in PCT International Application No. WO 2016/106302; WO 2016/011264; and WO 2009/126688.

TABLE 8
Exemplary Heavy Chain Sequences

| | | |
|---|---|---|
| Heavy | 291 | ASTKGPSVFPLAPSSKSTSGGTAALGCLVK |
| chain | | DYFPEPVTVSWNSGALTSGVHTFPAVLQSS |
| constant | | GLYSLSSVVTVPSSSLGTQTYICNVNHKPS |
| domain- | | NTKVDKKVEPKSCDKTHTCPPCPAPELLGG |
| IgG1 | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSRDE |
| | | LTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSPGK |
| Heavy | 292 | TKGPSVFPLAPCSRSTSESTAALGCLVKDY |
| chain | | FPEPVTVSWNSGALTSGVHTFPAVLQSSGL |
| constant | | YSLSSVVTVPSSSLGTKTYTCNVDHKPSNT |
| domain- | | KVDKRVESKYGPPCPPCPAPEFLGGPSVFL |
| IgG4 | | FPPKPKDTLMISRTPEVTCVVVDVSQEDPE |
| S228P | | VQFNWYVDGVEVHNAKTKPREEQFNSTYRV |
| | | VSVLTVLHQDWLNGKEYKCKVSNKGLPSSI |
| | | EKTISKAKGQPREPQVYTLPPSQEEMTKNQ |
| | | VSLTCLVKGFYPSDIAVEWESNGQPENNYK |
| | | TTPPVLDSDGSFFLYSRLTVDKSRWQEGNV |
| | | FSCSVMHEALHNHYTQKSLSLSLGK |
| Kappa | 293 | VAAPSVFIFPPSDEQLKSGTASVVCLLNNF |
| light | | YPREAKVQWKVDNALQSGNSQESVTEQDSK |
| chain | | DSTYSLSSTLTLSKADYEKHKVYACEVTHQ |
| constant | | GLSSPVTKSFNRGEC |
| domain | | |

In any of the above mentioned embodiments, the anti-TIGIT antibody or antigen binding fragment thereof is an antibody comprising any of the variable heavy chains described above and any human heavy chain constant domain. In one embodiment, the antibody or antigen binding fragment thereof of the invention is of the IgG isotype, and comprises a human IgG1, IgG2, IgG3 or IgG4 human heavy chain constant domain. In one embodiment, the antibody or antigen binding fragment thereof of the invention comprises a human heavy chain IgG1 constant domain (SEQ ID NO: 291) or a variant thereof, wherein the variant comprises up to 20 modified amino acid substitutions. In one embodiment, the antibody or antigen binding fragment thereof of the invention is an antibody comprising a human heavy chain IgG1 constant domain comprising the amino acid sequence as set forth in SEQ ID NO: 291. In one embodiment, the antibody or antigen binding fragment thereof of the invention comprises a human heavy chain IgG1 constant domain wherein the IgG1 constant domain is afucosylated. In one embodiment, the antibody or antigen binding fragment thereof of the invention comprises a human heavy chain IgG4 constant domain or a variant thereof, wherein the variant comprises up to 20 modified amino acid substitutions. In another embodiment, the antibody or antigen binding fragment thereof of the invention comprises a human heavy chain IgG4 constant domain, wherein the amino acid at position 228 (using EU numbering scheme) has been substituted from Ser to Pro. In one embodiment, the antibody or antigen binding fragment thereof of the invention comprises a human heavy chain IgG4 constant domain comprising the amino acid sequence as set forth in SEQ ID NO: 292.

In any of the above mentioned embodiments, the anti-TIGIT antibody or antigen binding fragment thereof can comprise any of the variable light chains described above and human light chain constant domain. In one embodiment, the antibody or antigen binding fragment thereof of the invention comprises a human kappa light chain constant domain or a variant thereof, wherein the variant comprises up to 20 modified amino acid substitutions. In another embodiment, the antibody or antigen binding fragment thereof of the invention comprises a human lambda light chain constant domain or a variant thereof, wherein the variant comprises up to 20 modified amino acid substitutions. In one embodiment, the antibody or antigen binding fragment thereof of the invention comprises a human kappa light chain constant domain comprising the amino acid sequence as set forth in SEQ ID NO: 293.

### Methods of Treating Cancer Using a Combination of a PD-1 Antagonist, a TIGIT Antagonist, and Lenvatinib or a Pharmaceutically Acceptable Salt Thereof.

In another aspect, provided herein are methods of treating cancer (e.g., endometrial cancer, HCC) using a combination of a TIGIT antagonist, a PD-1 antagonist, and lenvatinib or a pharmaceutically acceptable salt thereof.

In certain embodiments, the TIGIT antagonist is an anti-TIGIT antibody or antigen binding fragment thereof. In some embodiments, the PD-1 antagonist is an anti-PD-1 antibody or antigen binding fragment thereof.

In certain embodiments, the method of treating cancer comprises administering to a human patient in need thereof:
(a) a TIGIT antagonist;
(b) a PD-1 antagonist; and
(c) lenvatinib represented by Formula (I),

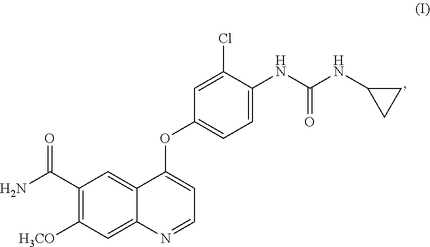 [SMILES: COc1cc2nccc(Oc3ccc(NC(=O)NC4CC4)c(Cl)c3)c2cc1C(N)=O]

or a pharmaceutically acceptable salt thereof.

In some embodiments, the cancer is selected from the group consisting of: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome); hematopoietic tumors of the lymphoid lineage, include leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoetic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma.

In some embodiments, the cancer is selected from the group consisting of anal cancer, biliary tract cancer, bladder cancer, breast cancer, cervical cancer, colon cancer, colorectal cancer (CRC), esophageal cancer, gastrointestinal cancer, glioblastoma, glioma, head and neck cancer (HNSCC), hepatocellular carcinoma (HCC), lung cancer, liver cancer, lymphoma, melanoma, mesothelioma, multiple myeloma, non-small cell lung cancer (NSCLC), ovarian cancer, pancreatic cancer, prostate cancer (e.g. hormone refractory prostate adenocarcinoma), renal cell carcinoma (RCC), salivary cancer, thyroid cancer, and other neoplastic malignancies.

In some embodiments, the cancer is selected from the group consisting of endometrial cancer, hepatocellular carcinoma (HCC), cervical cancer, head and neck cancer (HNSCC), biliary cancer, esophageal cancer, and triple negative breast cancer (TNBC).

In some embodiments, the cancer is selected from the group consisting of endometrial cancer, and hepatocellular carcinoma.

In some embodiments, the cancer is endometrial cancer. In some embodiments, the endometrial cancer is mismatch repair proficient (pMMR).

In some embodiments, the cancer is hepatocellular carcinoma. In some embodiments, the HCC is locally recurrent unresectable. In some embodiments, the HCC is metastatic.

A combination of a TIGIT antagonist, a PD-1 antagonist, and lenvatinib or a pharmaceutically acceptable salt thereof may provide enhanced efficacy as compared with existing treatments. For example, TIGIT and PD-L1 are co-expressed in HCC tumors. A combination of a TIGIT antagonist, a PD-1 antagonist, and lenvatinib or a pharmaceutically acceptable salt thereof may provide added benefit over single- or dual-agent checkpoint blockade. In certain embodiments, the tolerability of vibostolimab is a potential added benefit for combination with lenvatinib or a pharmaceutically acceptable salt thereof and a PD-1 antagonist.

In certain embodiments, the cancer is metastatic. In some embodiments, the cancer is relapsed. In other embodiments, the cancer is refractory. In yet other embodiments, the cancer is relapsed and refractory.

In certain embodiments, provided herein is a method of treating endometrial cancer, comprising administering to a human patient in need thereof
(a) a TIGIT antagonist;
(b) a PD-1 antagonist; and
(c) lenvatinib represented by Formula (I),

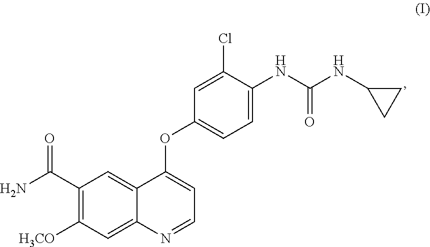 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is a method of treating hepatocellular carcinoma, comprising administering to a human patient in need thereof:
(a) a TIGIT antagonist;
(b) a PD-1 antagonist; and
(c) lenvatinib represented by Formula (I),

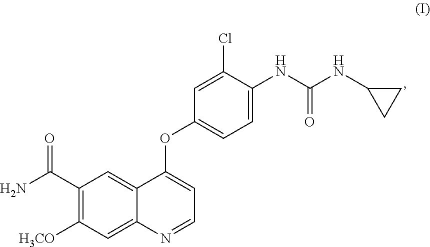 [SMILES: COc1cc2nccc(Oc3ccc(NC(=O)NC4CC4)c(Cl)c3)c2cc1C(N)=O]

or a pharmaceutically acceptable salt thereof.

In certain embodiments, the method of treating cancer comprises administering to a human patient in need thereof:
(a) a TIGIT antagonist as disclosed in Section titled TIGIT Antagonists;
(b) a PD-1 antagonist as disclosed in Section titled PD-1 Antagonists; and
(c) lenvatinib or a pharmaceutically acceptable salt thereof.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the TIGIT antagonist is an anti-human TIGIT monoclonal antibody or antigen binding fragment thereof. In some embodiments, the anti-human TIGIT monoclonal antibody is a human antibody. In other embodiments, the anti-human TIGIT monoclonal antibody is a humanized antibody.

Thus, in certain embodiments, provided herein is a method for treating cancer, comprising administering to a human patient in need thereof:
(a) a human or humanized anti-human TIGIT monoclonal antibody or antigen binding fragment thereof;
(b) a human or humanized anti-human PD-1 monoclonal antibody or antigen binding fragment thereof; and
(c) lenvatinib represented by Formula (I),

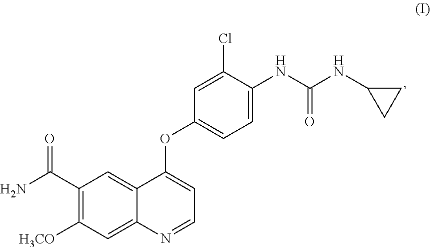 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is a method for treating cancer, comprising administering to a human patient in need thereof:
(a) a human anti-human TIGIT monoclonal antibody or antigen binding fragment thereof;
(b) a human anti-human PD-1 monoclonal antibody or antigen binding fragment thereof; and
(c) lenvatinib represented by Formula (I),

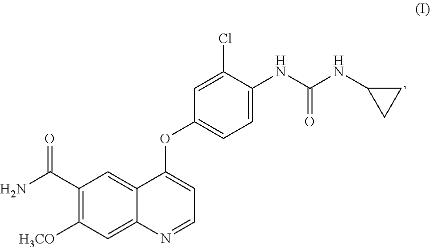 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is a method for treating cancer, comprising administering to a human patient in need thereof:
(a) a humanized anti-human TIGIT monoclonal antibody or antigen binding fragment thereof;
(b) a humanized anti-human PD-1 monoclonal antibody or antigen binding fragment thereof; and
(c) lenvatinib represented by Formula (I),

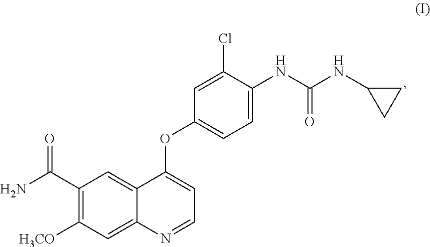 [SMILES: COc1cc2nccc(Oc3ccc(NC(=O)NC4CC4)c(Cl)c3)c2cc1C(N)=O]

or a pharmaceutically acceptable salt thereof.

In one embodiment of the methods provided herein, the anti-human PD-1 monoclonal antibody or antigen binding fragment thereof is pembrolizumab.

In another embodiment of the methods provided herein, the anti-human PD-1 monoclonal antibody or antigen binding fragment thereof is nivolumab.

In another embodiment of the methods provided herein, the anti-human PD-1 monoclonal antibody or antigen binding fragment thereof is cemiplimab.

In certain embodiments of the methods provided herein, the anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of the methods provided herein, the anti-TIGIT antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In certain embodiments, the anti-TIGIT antibody or antigen binding fragment thereof comprises a light chain comprising or consisting of the amino acid sequence as set forth in SEQ ID NO:294 and a heavy chain comprising or consisting of the amino acid sequence as set forth in SEQ ID NO:295.

In one specific embodiment of the methods, kits and uses provided herein, the method for treating cancer comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) lenvatinib, or a pharmaceutically acceptable salt thereof.

In one specific embodiment of the methods, kits and uses provided herein, the method for treating cancer comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) lenvatinib, or a pharmaceutically acceptable salt thereof.

In one specific embodiment of the methods, kits and uses provided herein, the method for treating cancer comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) lenvatinib, or a pharmaceutically acceptable salt thereof.

In one specific embodiment of the methods, kits and uses provided herein, the method for treating endometrial cancer comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) lenvatinib, or a pharmaceutically acceptable salt thereof.

In one specific embodiment of the methods, kits and uses provided herein, the method for treating endometrial cancer comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) lenvatinib, or a pharmaceutically acceptable salt thereof.

In one specific embodiment of the methods, kits and uses provided herein, the method for treating endometrial cancer comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) lenvatinib, or a pharmaceutically acceptable salt thereof.

In one specific embodiment of the methods, kits and uses provided herein, the method for treating HCC comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) lenvatinib, or a pharmaceutically acceptable salt thereof.

In one specific embodiment of the methods provided herein, the method for treating HCC comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) lenvatinib, or a pharmaceutically acceptable salt thereof.

In one specific embodiment of the methods provided herein, the method for treating HCC comprises administering to a human patient in need thereof:
(a) an anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) lenvatinib, or a pharmaceutically acceptable salt thereof.

### Dosing and Administration

Further provided herein are dosing regimens and routes of administration for treating cancer (e.g., endometrial cancer, HCC) using a combination of a TIGIT antagonist (e.g., an anti-TIGIT monoclonal antibody or antigen binding fragment thereof), a PD-1 antagonist (e.g., an anti-PD-1 monoclonal antibody or antigen binding fragment thereof), and a multi-RTK inhibitor (e.g., lenvatinib or a pharmaceutically acceptable salt thereof).

The anti-TIGIT antibody (e.g., anti-TIGIT monoclonal antibody) or antigen binding fragment thereof, the anti-PD-1 antibody (e.g., anti-PD-1 monoclonal antibody) or antigen binding fragment thereof, or lenvatinib or a pharmaceutically acceptable salt thereof disclosed herein may be administered by doses administered, e.g., daily, 1-7 times per week, weekly, biweekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. In certain embodiments, the doses are administered orally.

In some embodiments, the anti-TIGIT antibody (e.g., anti-TIGIT monoclonal antibody) thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at about 10, about 20, about 50, about 80, about 100, about 200, about 300, about 400, about 500, about 1000 or about 2500 mg/subject.

In some specific methods, the dose of the anti-TIGIT antibody (e.g., anti-TIGIT monoclonal antibody) or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 monoclonal antibody or antigen binding fragment thereof is about 50 mg, about 75 mg, about 100 mg, about 125 mg, about 150 mg, about 175 mg, about 200 mg, about 225 mg, about 240 mg, about 250 mg, about 300 mg, about 400 mg, or about 500 mg.

In some embodiments, the anti-PD-1 antibody (e.g., anti-PD-1 monoclonal antibody) or antigen binding fragment thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at about 10, about 20, about 50, about 80, about 100, about 200, about 300, about 400, about 500, about 1000 or about 2500 mg/subject.

In some specific methods, the dose of the anti-PD-1 antibody (e.g., anti-PD-1 monoclonal antibody) or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the anti-PD-1 antibody (e.g., anti-PD-1 monoclonal antibody) or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 antibody (e.g., anti-PD-1 monoclonal antibody) or antigen binding fragment thereof is about 50 mg, about 75 mg, about 100 mg, about 125 mg, about 150 mg, about 175 mg, about 200 mg, about 225 mg, about 240 mg, about 250 mg, about 300 mg, about 400 mg, or about 500 mg.

In some embodiments of the methods, compositions, kits and uses described herein, the anti-TIGIT antibody (e.g., anti-TIGIT monoclonal antibody) comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered about 100 mg, about 150 mg, about 200 mg, about 240 mg, about 400 mg, about 480 mg, or about 720 mg, or about 2 mg/kg anti-TIGIT antibody (e.g., anti-TIGIT monoclonal antibody), and the anti-TIGIT antibody (e.g., anti-TIGIT monoclonal antibody) is administered once every three or six weeks. In one embodiment, the human patient is administered about 200 mg anti-TIGIT antibody (e.g., anti-TIGIT monoclonal antibody) once every three weeks. In one embodiment, the human patient is administered 240 mg anti-TIGIT antibody (e.g., anti-TIGIT monoclonal antibody) once every three weeks. In one embodiment, the human patient is administered 2 mg/kg anti-TIGIT antibody (e.g., anti-TIGIT monoclonal antibody) once every three weeks. In one embodiment, the human patient is administered 400 mg anti-TIGIT antibody (e.g., anti-TIGIT monoclonal antibody) once every three weeks.

In certain embodiments of the methods described herein, the anti-TIGIT antibody (e.g., anti-TIGIT monoclonal antibody) comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered 400 mg anti-TIGIT antibody (e.g., anti-TIGIT monoclonal antibody), and the anti-TIGIT antibody (e.g., anti-TIGIT monoclonal antibody) is administered once every six weeks.

In some embodiments of the methods described herein, the anti-TIGIT antibody (e.g., anti-TIGIT monoclonal antibody) comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered 200 mg, 240 mg, 400 mg, 480 mg, 720 mg, or 2 mg/kg anti-TIGIT antibody (e.g., anti-TIGIT monoclonal antibody), and the anti-TIGIT monoclonal antibody is administered once every six weeks. In one embodiment, the human patient is administered 200 mg anti-TIGIT antibody (e.g., anti-TIGIT monoclonal antibody) once every six weeks. In one embodiment, the human patient is administered 240 mg anti-TIGIT antibody (e.g., anti-TIGIT monoclonal antibody) once every six weeks. In one embodiment, the human patient is administered 400 mg anti-TIGIT antibody (e.g., anti-TIGIT monoclonal antibody) once every six weeks. In one embodiment, the human patient is administered about 480 mg anti-TIGIT antibody (e.g., anti-TIGIT monoclonal antibody) once every six weeks. In one embodiment, the human patient is administered 720 mg anti-TIGIT antibody (e.g., anti-TIGIT monoclonal antibody) once every six weeks. In one embodiment, the human patient is administered 2 mg/kg anti-TIGIT antibody (e.g., anti-TIGIT monoclonal antibody) once every six weeks.

In some embodiments of the methods, compositions, kits and uses described herein, the anti-human PD-1 antibody (e.g., anti-PD-1 monoclonal antibody) or antigen binding fragment thereof is pembrolizumab, the human patient is administered about 200 mg, about 240 mg, about 400 mg, about 480 mg, about 720 mg, or about 2 mg/kg pembrolizumab, and pembrolizumab is administered once every three or six weeks. In one embodiment, the human patient is administered about 200 mg pembrolizumab once every three weeks. In one embodiment, the human patient is administered about 240 mg pembrolizumab once every three weeks. In one embodiment, the human patient is administered 2 mg/kg pembrolizumab once every three weeks. In one embodiment, the human patient is administered 400 mg pembrolizumab once every three weeks.

In certain embodiments of the methods, compositions, kits and uses described herein, the anti-human PD-1 monoclonal antibody or antigen binding fragment thereof is pembrolizumab, the human patient is administered 400 mg pembrolizumab, and pembrolizumab is administered once every six weeks.

In some embodiments of the methods, compositions, kits and uses described herein, the anti-human PD-1 monoclonal antibody or antigen binding fragment thereof is pembrolizumab, the human patient is administered about 200 mg, about 240 mg, about 400 mg, about 480 mg, about 720 mg, or about 2 mg/kg pembrolizumab, and pembrolizumab is administered once every six weeks. In one embodiment, the human patient is administered about 200 mg pembrolizumab once every six weeks. In one embodiment, the human patient is administered about 240 mg pembrolizumab once every six weeks. In one embodiment, the human patient is administered about 400 mg pembrolizumab once every six weeks. In one embodiment, the human patient is administered 480 mg pembrolizumab once every six weeks. In one embodiment, the human patient is administered 720 mg pembrolizumab once every six weeks. In one embodiment, the human patient is administered 2 mg/kg pembrolizumab once every six weeks.

In some embodiments of the invention, the anti-TIGIT antibody, or antigen binding fragment thereof, and the anti-PD-1 antibody, or antigen binding fragment thereof, are administered to the patient once every approximately six weeks for 12 weeks or more. In other embodiments, the anti-TIGIT antibody, or antigen binding fragment and the anti-PD-1 antibody, or antigen binding fragment thereof, are administered to the patient once every six weeks for 18 weeks or more, 24 weeks or more, 30 weeks or more, 36 weeks or more, 42 weeks or more, 48 weeks or more, 54 weeks or more, 60 weeks or more, 66 weeks or more, 72 weeks or more, 78 weeks or more, 84 weeks or more, or 90 weeks or more. In one embodiment, the administration occurs on the same day.

In a sub-embodiment, the anti-TIGIT antibody, or antigen binding fragment thereof, and the anti-PD-1 antibody, or antigen binding fragment thereof, are administered on the same day simultaneously (e.g., in a single formulation, a co-formulation or concurrently as separate formulations). In an alternative embodiment, the anti-TIGIT antibody or antigen binding fragment thereof and the anti-PD-1 antibody or antigen binding fragment thereof are administered sequentially on the same day (e.g., as separate formulations), in either order. In one embodiment of same day sequential administration, the anti-TIGIT antibody or antigen binding fragment thereof is administered first. In another embodiment of same day sequential administration, the anti-PD-1 antibody or antigen binding fragment thereof is administered first.

In certain embodiments of the methods, compositions, kits and uses described herein, the anti-human TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered 200 mg anti-human TIGIT antibody, and the anti-human TIGIT antibody is administered once every three weeks. In certain embodiments of the methods, compositions, kits and uses described herein, the anti-human TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered about 400 mg anti-human TIGIT antibody, and the anti-human TIGIT antibody is administered once every six weeks.

In certain embodiments of the methods, compositions, kits and uses described herein, the anti-human PD-1 monoclonal antibody or antigen binding fragment thereof is pembrolizumab, the human patient is administered about 200 mg pembrolizumab, and pembrolizumab is administered once every three weeks. In certain embodiments of the methods, compositions, kits and uses described herein, the anti-human PD-1 monoclonal antibody or antigen binding fragment thereof is pembrolizumab, the human patient is administered about 400 mg pembrolizumab, and pembrolizumab is administered once every six weeks.

In other embodiments of the methods, compositions, kits and uses described herein, the anti-human PD-1 monoclonal antibody or antigen binding fragment thereof is nivolumab, the human patient is administered about 240 mg or about 3 mg/kg nivolumab, and nivolumab is administered once every two weeks. In one specific embodiment, the human patient is administered about 240 mg nivolumab once every two weeks. In one specific embodiment, the human patient is administered about 3 mg/kg nivolumab once every two weeks. In other embodiments of the methods, compositions, kits and uses described herein, the anti-human PD-1 monoclonal antibody or antigen binding fragment thereof is nivolumab, the human patient is administered about 480 mg nivolumab, and nivolumab is administered once every four weeks.

In yet other embodiments of the methods, compositions, kits and uses described herein, the anti-human PD-1 monoclonal antibody or antigen binding fragment thereof is cemiplimab, the human patient is administered about 350 mg cemiplimab, and cemiplimab is administered once every three weeks.

In some embodiments, an anti-TIGIT antibody and anti-PD-1 antibody are co-formulated. In one embodiment, a co-formulated product with about 200 mg pembrolizumab or a pembrolizumab variant and 200 mg of antibody comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In one embodiment, a co-formulated product with 200 mg pembrolizumab or a pembrolizumab variant and 300 mg of antibody comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In one embodiment, a co-formulated product with about 200 mg pembrolizumab or a pembrolizumab variant and about 400 mg of antibody comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In another embodiment, a co-formulated product with about 200 mg of pembrolizumab or a pembrolizumab variant and about 500 mg of antibody comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In another embodiment, a co-formulated product with of about 200 mg pembrolizumab or a pembrolizumab variant and about 600 mg of antibody comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In another embodiment, a co-formulated product with about 200 mg of pembrolizumab or a pembrolizumab variant and about 700 mg of antibody comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion.

In certain embodiments, lenvatinib or a pharmaceutically acceptable salt thereof is administered orally. In some embodiments, lenvatinib or a pharmaceutically acceptable salt thereof is administered at a daily dose of about 4, about 8, about 10, about 12, about 14, about 18, about 20, or about 24 mg, of lenvatinib.

Thus, in some embodiments of the methods, compositions, kits and uses provided herein, the human patient is administered:
(a) about 100 mg, about 150 mg, about 200 mg, about 250 mg, or about 400 mg, or about 2 mg/kg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 100 mg, about 150 mg, about 200 mg, about 250 mg, or about 400 mg, or about 2 mg/kg pembrolizumab; and
(c) about 4, about 8, about 10, about 12, about 14, about 18, about 20, or about 24 mg lenvatinib;
wherein each (a) and (b) are administered once every three or six weeks, and wherein (c) is administered daily.

In certain embodiments of the methods, compositions, kits and uses provided herein, the human patient is administered:
(a) about 200 mg, about 240 mg, about 400 mg, about 2 mg/kg or about 22 mg/mL of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110; and
(b) about 200 mg, about 240 mg, about 400 mg, about 2 mg/kg or about 22 mg/mL pembrolizumab; and
(c) about 4, about 8, about 10, about 12, about 14, about 18, about 20, or about 24 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 20 mg lenvatinib;
wherein each of (a) and (b) is administered once every three weeks; and wherein (c) is administered daily.

In certain embodiments of the methods provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 14 mg lenvatinib;
wherein each of (a) and (b) is administered once every three weeks; and wherein (c) is administered daily.

In certain embodiments of the methods provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 12 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 8 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 4 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a heavy chain variable region comprising the amino acid sequence as set forth in SEQ ID NO: 148 and a light chain variable region comprising the amino acid sequence as set forth in SEQ ID NO: 152;
(b) about 200 mg pembrolizumab; and
(c) about 20 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a heavy chain variable region comprising the amino acid sequence as set forth in SEQ ID NO: 148 and a light chain variable region comprising the amino acid sequence as set forth in SEQ ID NO: 152;
(b) about 200 mg pembrolizumab; and
(c) about 14 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a heavy chain variable region comprising the amino acid sequence as set forth in SEQ ID NO: 148 and a light chain variable region comprising the amino acid sequence as set forth in SEQ ID NO: 152;
(b) about 200 mg pembrolizumab; and
(c) about 12 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a heavy chain variable region comprising the amino acid sequence as set forth in SEQ ID NO: 148 and a light chain variable region comprising the amino acid sequence as set forth in SEQ ID NO: 152;
(b) about 200 mg pembrolizumab; and
(c) about 8 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a heavy chain variable region comprising the amino acid sequence as set forth in SEQ ID NO: 148 and a light chain variable region comprising the amino acid sequence as set forth in SEQ ID NO: 152;
(b) about 200 mg pembrolizumab; and
(c) about 4 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295;
(b) about 200 mg pembrolizumab; and
(c) about 20 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295;
(b) about 200 mg pembrolizumab; and
(c) about 14 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295;
(b) about 200 mg pembrolizumab; and
(c) about 12 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295;
(b) about 200 mg pembrolizumab; and
(c) about 8 mg lenvatinib;
wherein each of (a) and (b) is administered once every three weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising a light chain comprising or consisting of the amino acid sequence as set forth in SEQ ID NO: 294 and a heavy chain comprising or consisting of the amino acid sequence as set forth in SEQ ID NO: 295;
(b) about 200 mg pembrolizumab; and
(c) about 4 mg lenvatinib;
wherein each of (a) and (b) is administered once every three weeks; and wherein (c) is administered daily.

In certain embodiments of the methods, compositions, kits and uses provided herein, the human patient is administered:
(a) about 240 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 240 mg pembrolizumab; and
(c) about 20 mg lenvatinib;
wherein each of (a) and (b) is administered once every three weeks; and wherein (c) is administered daily.

In certain embodiments of the methods, compositions, kits and uses provided herein, the human patient is administered:
(a) about 2 mg/kg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 2 mg/kg pembrolizumab; and
(c) about 20 mg lenvatinib;
wherein each of (a) and (b) is administered once every three weeks; and wherein (c) is administered daily.

In certain embodiments of the methods, compositions, kits and uses provided herein, the human patient is administered:
(a) about 400 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 400 mg pembrolizumab; and
(c) about 20 mg lenvatinib;
wherein (a) is administered once every six weeks; wherein (b) is administered once every six weeks; and wherein (c) is administered daily.

In certain embodiments of the methods, compositions, kits and uses provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks;
(b) about 400 mg pembrolizumab once every six weeks; and
(c) about 20 mg lenvatinib;
wherein (c) is administered daily. In some embodiments, (a) and (b) are co-formulated or co-administered.

In certain embodiments of the methods, compositions, kits and uses provided herein, the human patient is administered:
(a) about 200 mg of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks;
(b) about 400 mg pembrolizumab once every six weeks; and
(c) about 20 mg lenvatinib;
wherein (c) is administered daily.

In certain embodiments of the methods, compositions, kits and uses described herein, a co-formulation of pembrolizumab and of an anti-TIGIT antibody or antigen binding fragment thereof comprising three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 are administered by IV infusion. In some embodiments, the co-formulation is administered for about 30 minutes every three weeks. In some embodiments, the co-formulation is administered for from about 25 to about 40 minutes every three weeks.

In some embodiments, at least one of the therapeutic agents (e.g., the anti-TIGIT monoclonal antibody or binding fragment thereof, the anti-PD-1 monoclonal antibody or binding fragment thereof, or lenvatinib) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-TIGIT monoclonal antibody or binding fragment thereof, the anti-PD-1 monoclonal antibody or binding fragment thereof, or lenvatinib) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a biotherapeutic or chemotherapeutic agent, i.e., is treatment-naïve. In other embodiments, the combination therapy is administered to a patient who failed to achieve a sustained response after prior therapy with the biotherapeutic or chemotherapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with one or more of the therapeutic agents in the combinations disclosed herein.

The one or more additional active agents may be co-administered with the anti-TIGIT monoclonal antibody or antigen binding fragment thereof, the anti-PD-1 monoclonal antibody or antigen binding fragment thereof, or lenvatinib or a pharmaceutically acceptable salt thereof. The additional active agent(s) can be administered in a single dosage form with one or more co-administered agent selected from the anti-TIGIT monoclonal antibody or antigen binding fragment thereof, the anti-PD-1 monoclonal antibody or antigen binding fragment thereof, and lenvatinib or a pharmaceutically acceptable salt thereof. The additional active agent(s) can also be administered in separate dosage form(s) from the dosage forms containing the anti-TIGIT monoclonal antibody or antigen binding fragment thereof, the anti-PD-1 monoclonal antibody or antigen binding fragment thereof, or lenvatinib or a pharmaceutically acceptable salt thereof.

### Pharmaceutical Compositions

In yet another aspect, provided herein are pharmaceutical compositions comprising the therapeutic agents disclosed herein (e.g., a TIGIT antagonist, a PD-1 antagonist, and lenvatinib).

In certain embodiments, the pharmaceutical composition further comprises a pharmaceutically acceptable carrier.

The pharmaceutical compositions comprising an anti-human TIGIT antibody (e.g., anti-TIGIT monoclonal antibody) or antigen binding fragment thereof, an anti-human PD-1 antibody (e.g., anti-PD-1 monoclonal antibody) or antigen binding fragment thereof, and lenvatinib or pharmaceutically acceptable salt thereof, can be prepared for storage by mixing the antibodies or compounds having the desired degree of purity with optionally physiologically acceptable carriers, excipients, or stabilizers (see, e.g., Remington, Remington's Pharmaceutical Sciences (18^{th} ed. 1980)) in the form of aqueous solutions or lyophilized or other dried forms.

The pharmaceutically acceptable carriers, excipients, or stabilizers are non-toxic to the cell or mammalian being exposed thereto at the dosage and concentrations employed. Often the pharmaceutically acceptable carrier is an aqueous pH buffered solution. Examples of pharmaceutically acceptable carriers include buffers, such as phosphate, citrate, acetate, and other organic acids; antioxidants, such as ascorbic acid; low molecular weight (e.g., fewer than about 10 amino acid residues) polypeptide; proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The pharmaceutically acceptable carriers can also refer to a diluent, adjuvate (e.g., Freund's adjuvate (complete or incomplete)), excipient, or vehicle. Such carriers can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsions, tablets, pills, capsules, powders, sustained-release formulations, and the like.

### Kits

In still another aspect, provided herein are kits comprising the therapeutic agents disclosed herein (e.g., a TIGIT antagonist, a PD-1 antagonist, and lenvatinib) or pharmaceutical compositions thereof, packaged into suitable packaging material. A kit optionally includes a label or packaging insert that include a description of the components or instructions for use in vitro, in vivo, or ex vivo, of the components therein.

In some embodiments, the kit comprises
(a) a TIGIT antagonist;
(b) a PD-1 antagonist; and
(c) lenvatinib or a pharmaceutically acceptable salt thereof.

In certain embodiments, the kit further comprises instructions for administering to a human patient the TIGIT antagonist, the PD-1 antagonist, and lenvatinib or a pharmaceutically acceptable salt thereof.

In some embodiments, the TIGIT antagonist is an anti-TIGIT monoclonal antibody or antigen-binding fragment thereof. In some embodiments, the PD-1 antagonist is an anti-PD-1 monoclonal antibody or antigen-binding fragment thereof. In some embodiments, the PD-1 antagonist is an anti-PD-L1 monoclonal antibody or antigen-binding fragment thereof.

In one embodiment, the kit comprises: (a) one or more dosages of an anti-TIGIT antibody (e.g., anti-TIGIT monoclonal antibody) or antigen binding fragment thereof; (b) one or more dosages of an anti-PD-1 antibody (e.g., anti-PD-1 monoclonal antibody or antigen binding fragment thereof; (c) one or more dosages of lenvatinib or a pharmaceutically acceptable salt thereof; and (d) instructions for administering to a human patient the anti-human TIGIT antibody (e.g., anti-TIGIT monoclonal antibody) or antigen binding fragment thereof, the anti-human PD-1 antibody (e.g., anti-PD-1 monoclonal antibody) or antigen binding fragment thereof, and lenvatinib or a pharmaceutically acceptable salt thereof.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

The dosages for the anti-TIGIT antibody (e.g., anti-TIGIT monoclonal antibody), the anti-PD-1 antibody (e.g., anti-PD-1 monoclonal antibody), or lenvatinib or a pharmaceutically acceptable salt thereof described herein can be used in the kits herein. In some embodiments, a kit comprises dosages of each component sufficient for a certain period of treatment (e.g., 3, 6, 12, or 24 weeks, etc.). For example, a kit can comprise a dosage of about 200 mg pembrolizumab, a dosage of about 200 mg anti-TIGIT antibody, and 21 dosages of about 20 mg lenvatinib (or equivalent amount of a pharmaceutically acceptable salt of lenvatinib), which are sufficient for a 3-week treatment. Or, a kit can also comprise a dosage of about 400 mg pembrolizumab, 1 dosage of about 400 mg anti-TIGIT antibody, and 42 dosages of about 20 mg lenvatinib (or equivalent amount of a pharmaceutically acceptable salt of lenvatinib), which are sufficient for a 6-week treatment.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### Uses of a Therapeutic Combination for Treating Cancer

In still another aspect, provided herein are uses of a therapeutic combination for treating cancer (e.g., endometrial cancer, HCC) in a human patient, wherein the therapeutic combination comprises:
(a) a TIGIT antagonist;
(b) a PD-1 antagonist; and
(c) lenvatinib represented by Formula (I),

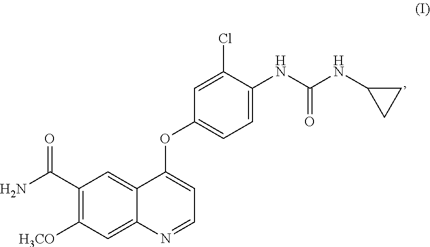 [SMILES: COc1cc2nccc(Oc3ccc(NC(=O)NC4CC4)c(Cl)c3)c2cc1C(N)=O]

or a pharmaceutically acceptable salt thereof.

In some embodiments, the cancer is selected from the group consisting of endometrial cancer, hepatocellular carcinoma (HCC), cervical cancer, head and neck cancer (HNSCC), biliary cancer, esophageal cancer, and triple negative breast cancer (TNBC).

In certain embodiments, the cancer is metastatic. In some embodiments, the cancer is relapsed. In other embodiments, the cancer is refractory. In yet other embodiments, the cancer is relapsed and refractory.

In one embodiment, the cancer is endometrial cancer. In one embodiment, the cancer is HCC.

In one embodiment, provided herein is use of a therapeutic combination for treating endometrial cancer in a human patient, wherein the therapeutic combination comprises:
(a) a TIGIT antagonist;
(b) a PD-1 antagonist; and
(c) lenvatinib represented by Formula (I),

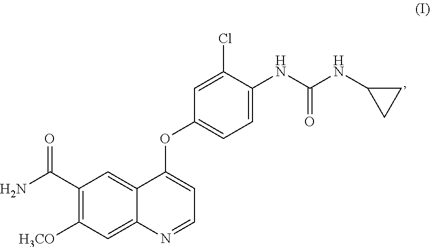 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating HCC in a human patient, wherein the therapeutic combination comprises:
(a) a TIGIT antagonist;
(b) a PD-1 antagonist; and
(c) lenvatinib represented by Formula (I),

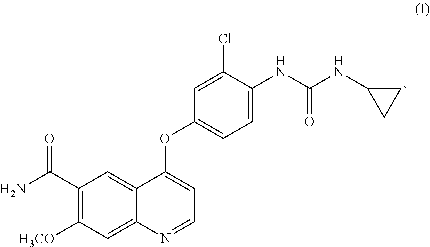 [SMILES: COc1cc2nccc(Oc3ccc(NC(=O)NC4CC4)c(Cl)c3)c2cc1C(N)=O]

or a pharmaceutically acceptable salt thereof.

In still other embodiments, provided herein is use of a therapeutic combination for treating cancer, wherein the therapeutic combination comprises:
(a) a TIGIT antagonist as disclosed in the present disclosure;
(b) a PD-1 antagonist as disclosed in the present disclosure; and
(c) lenvatinib represented by Formula (I),

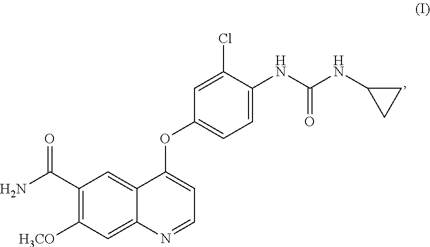 [SMILES: COc1cc2nccc(Oc3ccc(NC(=O)NC4CC4)c(Cl)c3)c2cc1C(N)=O]

or a pharmaceutically acceptable salt thereof.

In certain embodiments, the TIGIT antagonist is an anti-human TIGIT monoclonal antibody or antigen binding fragment thereof. In some embodiments, the anti-human TIGIT monoclonal antibody is a human antibody. In other embodiments, the anti-human TIGIT monoclonal antibody is a humanized antibody.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

Thus, in certain embodiments, provided herein is use of a therapeutic combination for treating cancer, wherein the therapeutic combination comprises:
(a) a human or humanized anti-human TIGIT monoclonal antibody or antigen binding fragment thereof;
(b) a human or humanized anti-human PD-1 monoclonal antibody or antigen binding fragment thereof; and
(c) lenvatinib represented by Formula (I),

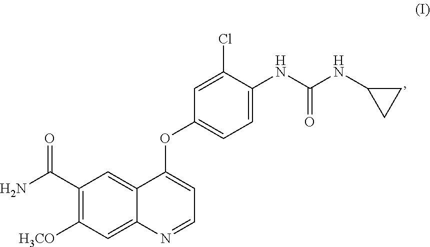 [SMILES: COc1cc2nccc(Oc3ccc(NC(=O)NC4CC4)c(Cl)c3)c2cc1C(N)=O]

or a pharmaceutically acceptable salt thereof.

In some embodiments, provided herein is use of a therapeutic combination for treating cancer, wherein the therapeutic combination comprises:
(a) a human anti-human TIGIT monoclonal antibody or antigen binding fragment thereof;
(b) a human anti-human PD-1 monoclonal antibody or antigen binding fragment thereof; and
(c) lenvatinib represented by Formula (I),

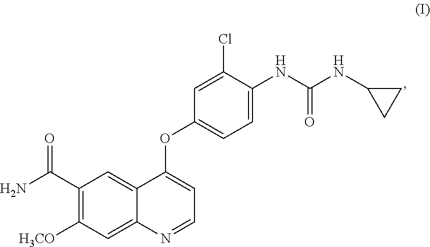 [SMILES: COc1cc2nccc(Oc3ccc(NC(=O)NC4CC4)c(Cl)c3)c2cc1C(N)=O]

or a pharmaceutically acceptable salt thereof.

In other embodiments, provided herein is use of a therapeutic combination for treating cancer, wherein the therapeutic combination comprises:
(a) a humanized anti-human TIGIT monoclonal antibody or antigen binding fragment thereof;
(b) a humanized anti-human PD-1 monoclonal antibody or antigen binding fragment thereof; and
(c) lenvatinib represented by Formula (I),

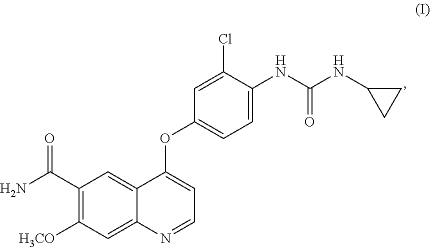 [SMILES: COc1cc2nccc(Oc3ccc(NC(=O)NC4CC4)c(Cl)c3)c2cc1C(N)=O]

or a pharmaceutically acceptable salt thereof.

In some embodiments of the uses provided herein, the anti-PD-1 monoclonal antibody or antigen binding fragment thereof is pembrolizumab. In some embodiments of the uses provided herein, the anti-PD-1 monoclonal antibody or antigen binding fragment thereof is nivolumab. In some embodiments of the uses provided herein, the anti-PD-1 monoclonal antibody or antigen binding fragment thereof is cemiplimab.

In certain embodiments of various uses provided herein, the anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of various uses provided herein, the anti-TIGIT antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In other embodiments of various uses provided herein, the anti-human TIGIT monoclonal antibody or antigen binding fragment thereof comprises a light chain comprising or consisting of the amino acid sequence as set forth in SEQ ID NO:294 and a heavy chain comprising or consisting of the amino acid sequence as set forth in SEQ ID NO:295.

Thus, in one specific embodiment, provided herein is use of a therapeutic combination for treating cancer, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) lenvatinib, or a pharmaceutically acceptable salt thereof.

In one specific embodiment, provided herein is use of a therapeutic combination for treating cancer, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) lenvatinib, or a pharmaceutically acceptable salt thereof.

In one specific embodiment, provided herein is use of a therapeutic combination for treating cancer, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) lenvatinib, or a pharmaceutically acceptable salt thereof.

In one specific embodiment, provided herein is use of a therapeutic combination for treating endometrial cancer, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) lenvatinib, or a pharmaceutically acceptable salt thereof.

In one specific embodiment, provided herein is use of a therapeutic combination for treating endometrial cancer, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) lenvatinib, or a pharmaceutically acceptable salt thereof.

In one specific embodiment, provided herein is use of a therapeutic combination for treating endometrial cancer, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) lenvatinib, or a pharmaceutically acceptable salt thereof.

In one specific embodiment, provided herein is use of a therapeutic combination for treating HCC, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) lenvatinib, or a pharmaceutically acceptable salt thereof.

In one specific embodiment, provided herein is use of a therapeutic combination for treating HCC, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) lenvatinib, or a pharmaceutically acceptable salt thereof.

In one specific embodiment, provided herein is use of a therapeutic combination for treating HCC, wherein the therapeutic combination comprises:
(a) anti-TIGIT antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) lenvatinib, or a pharmaceutically acceptable salt thereof.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000)Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and Fourier-transform infrared spectroscopy (FTIR). SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLES

The examples in this section are offered by way of illustration, and not by way of limitation.

### Example 1: Clinical Trial of Administering a Combination of an Anti-TIGIT Antibody and an Anti-PD-1 Antibody with Lenvatinib in Endometrial Cancer or HCC Patients

This study is a multicenter, open-label phase 2 study of an anti-TIGIT antagonist and an anti-PD-1 antagonist with or without lenvatinib for the treatment of endometrial cancer or HCC.

For endometrial cancer, approximately 80 participants with advanced endometrial cancer will be allocated by nonrandom assignment to study treatment based on mismatch repair (MMR) status. Participants whose tumors are mismatch repair deficient (dMMR) (n=40) will be allocated to Composition A (co-formulation of pembrolizumab/vibostolimab; vibostolimab comprising heavy chain comprising the amino acid sequence as set forth in SEQ ID NO: 295 and light chain comprising the amino acid sequence as set forth in SEQ ID NO: 294). Participants whose tumors are mismatch repair proficient (pMMR) (n=40) will be allocated to Composition A plus lenvatinib. A total of 40 participants will be allocated to receive Composition A plus the recommended Phase 2 dose (RP2D) for lenvatinib.

For HCC, approximately 40 participants with locally recurrent unresectable or metastatic HCC will be allocated by nonrandom assignment to Composition A+lenvatinib. A total of 40 participants will be allocated to receive Composition A plus the RP2D for lenvatinib.

### Objective and Endpoints

A listing of objectives and endpoints for the clinical trial substudy are shown in the table below.

TABLE 9
Objectives and endpoints for the clinical trial

| Objectives | Endpoints |
|---|---|
| Primary | |
| Objective: To evaluate Composition A | Objective response: |
| alone or in combination with other | A confirmed complete |
| anticancer therapies (e.g., Lenvatinib) with | response (CR) or |
| respect to objective response rate (ORR) | partial response (PR) |
| per Response Evaluation Criteria in Solid | |
| Tumors (RECIST) 1.1 as assessed by the | |
| investigator in participants with selected | |
| solid tumors, excluding those with cervical | |
| cancer whose tumors express PD-L1 | |
| (combined positive score (CPS) ≥1). | |
| Secondary | |
| Objective: To evaluate Composition A | OS: The time from |
| alone or in combination with other | randomization/ |
| anticancer therapies (e.g., Lenvatinib) with | treatment allocation to |
| respect to overall survival (OS) in | death due to any cause. |
| participants with selected solid tumors | |
| Objective: To evaluate Composition A | PFS: The time from |
| alone or in combination with other | randomization or first |
| anticancer therapies (e.g., Lenvatinib) with | documented disease |
| respect to progression-free survival (PFS) | progression or death |
| per RECIST 1.1 as assessed by the | due to any cause, |
| investigator in participants with selected | whichever occurs first. |
| solid tumors, excluding those with cervical | |
| cancer whose tumors express PD-L1 (CPS | |
| ≥1) | |
| Objective: To evaluate Composition A | DOR: For participants |
| alone or in combination with other | with a confirmed |
| anticancer therapies (e.g., Lenvatinib) with | CR or PR, DOR is |
| respect to duration of response (DOR) per | defined as the time |
| RECIST 1.1 as assessed by the investigator | from first documented |
| in participants with selected solid tumors, | evidence of CR or |
| excluding those with cervical cancer whose | PR until disease |
| tumors express PD-L1 (CPS ≥1) | progression or death due |
| | to any cause, whichever |
| | occurs first. |
| To evaluate the safety and tolerability of | AEs |
| Composition A alone or in combination | Discontinuations of |
| with other anticancer therapies (e.g., | study intervention due |
| Lenvatinib) | to AEs |

### Dosing

A modified Toxicity Probability Interval (mTPI) design with a target dose-limiting toxicity (DLT) rate of 3000 will be used to identify the preliminary RP2D of lenvatinib to use in combination with pembrolizumab and vibostolimab for the treatment of participants with pMMR endometrial cancer. If de-escalation is called for by mTPI, the dose of lenvatinib will be reduced while the dose of pembrolizumab and vibostolimab will remain fixed.

Treatment with Composition A will continue for a total of 35 administrations (approximately 2 years) or until any of the discontinuation criteria are met. Treatment with the RP2D of lenvatinib will continue until any of the discontinuation criteria are met.

There are 3 predetermined dose levels of lenvatinib for endometrial cancer that may be explored in the mTPI design (Table 10). If the mTPI calls for an additional de-escalation, enrollment will be put on hold while the Sponsor evaluates the observed dose-limiting toxicities (DLTs).

TABLE 10
Predetermined Dose Levels for Determining the Recommended
Phase 2 Dose in pMMR Endometrial Cancer

| Study | Dose | Dose | Dose |
|---|---|---|---|
| Intervention | Level 0 | Level −1 | Level −2 |
| Pembrolizumab | 200 mg Q3W | 200 mg Q3W | 200 mg Q3W |
| Vibostolimab | 200 mg Q3W | 200 mg Q3W | 200 mg Q3W |
| Lenvatinib | 20 mg qd | 14 mg qd | 10 mg qd |

Abbreviations: qd = once daily; Q3W = every 3 weeks

TABLE 11
Predetermined Dose Levels for Determining the
Recommended Phase 2 Dose in HCC

| Study Intervention | Dose Level 0 | Dose Level −1 |
|---|---|---|
| Pembrolizumab | 200 mg Q3W | 200 mg Q3W |
| Vibostolimab | 200 mg Q3W | 200 mg Q3W |
| Lenvatinib | 12 mg qd (BW ≥60 kg) | 8 mg qd (BW ≥60 kg) |
| | 8 mg qd (BW <60 kg) | 4 mg qd (BW <60 kg) |

Abbreviations: BW = body weight; qd = once daily.

Ten participants are planned at the starting doses of lenvatinib (Table 10 and Table 11). However, depending on the accrual rate, the completion of DLT evaluation period and number of DLTs observed, fewer participants may be enrolled at the starting dose. Participants will be closely followed for DLTs for the first cycle after the first dose of study intervention (the DLT evaluation period). The dose will be evaluated on an ongoing basis as participants complete the DLT evaluation period. In Table 12, the columns indicate the numbers of participants treated at the current dose level, and the rows indicate the numbers of participants experiencing a DLT. The entries of the table are the dose-finding decisions: E, S, D, and DU represent escalating the dose, staying at the same dose, de-escalating the dose, and excluding the dose from the study due to unacceptable toxicity, respectively. For example, if 2 out of 3 participants at this dose level develop a DLT, the dose will be de-escalated to the next lower dose level but may be re-escalated at a later time if the lower dose is well tolerated. If 3 out of 3 participants develop a DLT, this indicates an unacceptable toxicity at this dose. The dose should be de-escalated, if allowed per-protocol, and the current dose will not be explored further.

TABLE 12
Dose-finding Rules per mTPI Design (Target Toxicity Rate up to 30%)

| Number of participants | Number of Participants Evaluable for DLT at Current Dose | | | | | | | |
|---|---|---|---|---|---|---|---|---|
| with at least 1 DLT | 3 | 4 | 5 | 6 | 7 | 8 | 9 | 10 |
| 0 | E | E | E | E | E | E | E | E |
| 1 | S | S | S | E | E | E | E | E |
| 2 | D | S | S | S | S | S | S | S |
| 3 | DU | DU | D | S | S | S | S | S |
| 4 | | DU | DU | DU | D | D | S | S |
| 5 | | | DU | DU | DU | DU | DU | D |
| 6 | | | | DU | DU | DU | DU | DU |
| 7 | | | | | DU | DU | DU | DU |
| 8 | | | | | | DU | DU | DU |
| 9 | | | | | | | DU | DU |
| 10 | | | | | | | | DU |

Abbreviations: D = Deescalate to the next lower dose; DU = The current dose is unacceptably toxic; E = Escalate to the next higher dose; S = Stay at the current dose.
Note:
Target toxicity rate = up to 30% (inclusive). Flat noninformative prior Beta (1, 1) is used as a prior and ε1 = ε2 = 0.05 {03TFYL, 03FL3C, 04WC92}.
Note:
In this study, the dose will not be escalated above Dose Level 0, so an “Escalate” can also mean “Stay”.

If either a dose de-escalation or re-escalation decision is made before 10 participants have completed enrollment at the starting dose, the enrollment at this dose level will be halted and a new set of participants will be enrolled and treated at the next lower dose level. This process will continue, with up to 10 DLT-evaluable participants enrolled at each dose explored, until 10 participants have been enrolled at any of the tested doses and a dosing decision of “S” (for staying at current dose) is made for the 10th participant evaluated. If, at any point, the dosing decision is “E” after evaluation of all enrolled patients at a dose, the dose may be escalated to a higher predetermined dose provided that the higher dose has not previously been discontinued due to a “DU” decision.

A “D” or “DU” decision at the lowest dose level will stop the evaluation of the combination under study. An “E” decision at the highest dose level will result in staying at that level. During dose-finding, it may be acceptable to de-escalate or escalate to an intermediate dose that was not predefined and not previously studied if evaluation of toxicity at such a dose is desired.

The maximum dose/exposure allowed in this study is 200 mg vibostolimab/200 mg pembrolizumab for up to 2 years (35 treatment cycles) of initial treatment/first course.

The maximum dose of lenvatinib for participants in this study with pMMR tumors is 20 mg/day. The maximum dose of lenvatinib is 12 mg/day (BW≥60 kg) or 8 mg/day (BW<60 kg). Lenvatinib may be continued until a discontinuation criterion is met.

Dose modifications in response to treatment-related adverse events (AEs) are permitted to keep the participant on study medication, when appropriate. In general, Composition A, and lenvatinib may be interrupted together or separately at the Investigator's discretion to determine which treatment component is the source for a given toxicity. Action may be taken independently for each component of study treatment (e.g., lenvatinib should be discontinued for prohibitive toxicity that is lenvatinib-related, but Composition A may continue if well tolerated). Lenvatinib may be interrupted, dose reduced or discontinued. Composition A may only be interrupted or discontinued.

AEs associated with Composition A exposure may represent an immune-related response. These immune-related AEs (irAEs) may occur shortly after the first dose or several months after the last dose of pembrolizumab monotherapy, coformulation, or immune-oncology (IO) combination treatment and may affect more than one body system simultaneously. Therefore, early recognition and initiation of treatment is critical to reduce complications. Based on existing clinical study data, most irAEs were reversible and could be managed with interruptions of pembrolizumab monotherapy, coformulation, or id combination administration of corticosteroids and/or other supportive care. For suspected irAEs, ensure adequate evaluation to confirm etiology or exclude other causes. Additional procedures or tests such as bronchoscopy, endoscopy, skin biopsy may be included as part of the evaluation.

TABLE 13
Dose Modification and Toxicity Management Guidelines
for Immune-related Adverse Events Associated with
Pembrolizumab Monotherapy, Coformulations or IO Combinations
General instructions:
1. Severe and life-threatening irAEs should be treated with
IV corticosteroids followed by oral steroids. Other
immunosuppressive treatment should begin if the irAEs
are not controlled by corticosteroids.
2. Pembrolizumab monotherapy, coformulations or IO
combinations must be permanently discontinued if the
irAE does not resolve or the corticosteroid dose is not
≤10 mg/day within 12 weeks of the last treatment.
3. The corticosteroid taper should begin when the
irAE is ≤ Grade 1 and continue at least 4 weeks.
4. If pembrolizumab monotherapy, coformulations or IO
combinations have been withheld, treatment may
resume after the irAE decreased to ≤ Grade 1 after
corticosteroid taper.

| | | Action With | | |
|---|---|---|---|---|
| | | Pembrolizumab | | |
| | Toxicity | Monotherapy, | | |
| | Grade | Coformulations | Corticosteroid | |
| | (CTCAE | or IO | and/or Other | |
| irAEs | v5.0) | Combinations | Therapies | Monitoring and Follow-up |
| Pneumonitis | Grade 2 | Withhold | Administer | Monitor participants for signs and |
| | Recurrent | Permanently | corticosteroids | symptoms of pneumonitis |
| | Grade 2, | discontinue | (initial dose of 1 to | Evaluate participants with |
| | Grade 3 or | | 2 mg/kg | suspected pneumonitis with |
| | 4 | | prednisone or | radiographic imaging and initiate |
| | | | equivalent) | corticosteroid treatment |
| | | | followed by taper | |
| | | | Add prophylactic | |
| | | | antibiotics for | |
| | | | opportunistic | |
| | | | infections | |
| Diarrhea/Colitis | Grade 2 or | Withhold | Administer | Monitor participants for signs and |
| | 3 | | corticosteroids | symptoms of enterocolitis (i.e., |
| | Recurrent | Permanently | (initial dose of 1 to | diarrhea, abdominal pain, blood or |
| | Grade 3 or | discontinue | 2 mg/kg prednisone | mucus in stool with or without |
| | Grade 4 | | or equivalent) | fever) and of bowel perforation |
| | | | followed by taper | (i.e., peritoneal signs and ileus) |
| | | | | Participants with ≥ Grade 2 diarrhea |
| | | | | suspecting colitis should consider |
| | | | | GI consultation and performing |
| | | | | endoscopy to rule out colitis |
| | | | | Participants with diarrhea/colitis |
| | | | | should be advised to drink liberal |
| | | | | quantities of clear fluids. If |
| | | | | sufficient oral fluid intake is not |
| | | | | feasible, fluid and electrolytes |
| | | | | should be substituted via IV |
| | | | | infusion |
| AST or ALT | Grade 2 ^{a} | Withhold | Administer | Monitor with liver function tests |
| Elevation or | | | corticosteroids | (consider weekly or more |
| Increased | | | (initial dose of 0.5 | frequently until liver enzyme value |
| Bilirubin | | | to 1 mg/kg | returned to baseline or is stable) |
| | | | prednisone or | |
| | | | equivalent) | |
| | | | followed by taper | |
| | Grade 3 ^{b} | Permanently | Administer | |
| | or 4 ^{c} | discontinue | corticosteroids | |
| | | | (initial dose of 1 to | |
| | | | 2 mg/kg prednisone | |
| | | | or equivalent) | |
| | | | followed by taper | |
| TIDM or | New onset | Withhold ^{d} | Initiate insulin | Monitor participants for |
| Hyperglycemia | TIDM or | | replacement | hyperglycemia or other signs and |
| | Grade 3 or | | therapy for | symptoms of diabetes |
| | 4 | | participants with | |
| | hyperglycemia | | TIDM | |
| | associated | | Administer | |
| | with | | antihyperglycemic | |
| | evidence of | | in participants with | |
| | β-cell | | hyperglycemia | |
| | failure | | | |
| Hypophysitis | Grade 2 | Withhold | Administer | Monitor for signs and symptoms of |
| | Grade 3 or | Withhold or | corticosteroids and | hypophysitis (including |
| | 4 | permanently | initiate hormonal | hypopituitarism and adrenal |
| | | discontinue ^{d} | replacements as | insufficiency) |
| | | | clinically indicated | |
| Hyperthyroidism | Grade 2 | Continue | Treat with | Monitor for signs and symptoms of |
| | Grade 3 or | Withhold or | nonselective beta- | thyroid disorders |
| | 4 | permanently | blockers (e.g., | |
| | | discontinue ^{d} | propranolol) or | |
| | | | thionamides as | |
| | | | appropriate | |
| Hypothyroidism | Grade 2, 3 | Continue | Initiate thyroid | Monitor for signs and symptoms of |
| | or 4 | | replacement | thyroid disorders |
| | | | hormones (e.g., | |
| | | | levothyroxine or | |
| | | | liothyronine) per | |
| | | | standard of care | |
| Nephritis: | Grade 2 | Withhold | Administer | Monitor changes of renal function |
| grading | Grade 3 or | Permanently | corticosteroids | |
| according to | 4 | discontinue | (prednisone 1 to | |
| increased | | | 2 mg/kg or | |
| creatinine or | | | equivalent) | |
| acute kidney | | | followed by taper | |
| injury | | | | |
| Neurological | Grade 2 | Withhold | Based on severity | Ensure adequate evaluation to |
| Toxicities | Grade 3 or | Permanently | of AE administer | confirm etiology and/or exclude |
| | 4 | discontinue | corticosteroids | other causes |
| Myocarditis | Grade 1 | Withhold | Based on severity | Ensure adequate evaluation to |
| | Grade 2, 3 | Permanently | of AE administer | confirm etiology and/or exclude |
| | or 4 | discontinue | corticosteroids | other causes |
| Exfoliative | Suspected | Withhold | Based on severity | Ensure adequate evaluation to |
| Dermatologic | SJS, TEN, | | of AE administer | confirm etiology or exclude other |
| Conditions | or DRESS | | corticosteroids | causes |
| | Confirmed | Permanently | | |
| | SJS, TEN, | discontinue | | |
| | or DRESS | | | |
| All Other | Persistent | Withhold | Based on severity | Ensure adequate evaluation to |
| irAEs | Grade 2 | | of AE administer | confirm etiology or exclude other |
| | Grade 3 | Withhold or | corticosteroids | causes |
| | | discontinue | | |
| | | based on the | | |
| | | event ^{e} | | |
| | Recurrent | Permanently | | |
| | Grade 3 or | discontinue | | |
| | Grade 4 | | | |

AE(s) = adverse event(s);
ALT = alanine aminotransferase;
AST = aspartate aminotransferase;
CTCAE = Common Terminology Criteria for Adverse Events;
DRESS = Drug Rash with Eosinophilia and Systemic Symptom;
GI = gastrointestinal;
IO = immuno-oncology;
ir = immune related;
IV = intravenous;
SJS = Stevens-Johnson Syndrome;
T1DM = type 1 diabetes mellitus;
TEN = Toxic Epidermal Necrolysis;
ULN = upper limit of normal.
Note:
Non-irAE will be managed as appropriate, following clinical practice recommendations.
^{a} AST/ALT: >3.0 to5.0 × ULN if baseline normal; >3.0 to 5.0 × baseline, if baseline abnormal; bilirubin: >1.5 to 3.0 × ULN if baseline normal; >1.5 to 3.0 × baseline if baseline abnormal
^{b} AST/ALT: >5.0 to 20.0 × ULN, if baseline normal; >5.0 to 20.0 × baseline, if baseline abnormal; bilirubin: >3.0 to 10.0 × ULN if baseline normal; >3.0 to 10.0 × baseline if baseline abnormal
^{c} AST/ALT: >20.0 × ULN, if baseline normal; >20.0 × baseline, if baseline abnormal; bilirubin: >10.0 × ULN if baseline normal; >10.0 × baseline if baseline abnormal
^{d} The decision to withhold or permanently discontinue pembrolizumab monotherapy, coformulations or IO combinations is at the discretion of the investigator or treating physician. If control achieved or ≤ Grade 2, pembrolizumab monotherapy, coformulations or IO combinations may be resumed.
^{e} Events that require discontinuation include, but are not limited to: encephalitis and other clinically important irAEs (e.g., vasculitis and sclerosing cholangitis).

Based on the known toxicity profiles of Composition A and lenvatinib, certain treatment-related AEs are uniquely associated with one drug versus the other. For example, hypertension, arterial thrombotic events, proteinuria, and hemorrhagic events are known risks for lenvatinib treatment, while immune-related AEs are risks for Composition A treatment. However, certain AEs, such as such as diarrhea, hypothyroidism, and liver enzyme elevation, may be initially considered attributable to either study drug. Therefore, evaluation of attribution is important for determining the study drug most likely related to the AE, or an alternative etiology, and subsequently proper clinical management. The following aspects should be considered:
1. Timing of AE Onset
Since lenvatinib is dosed daily and continuously due to a relatively short half-life (28 hours), and Composition A is dosed Q3W due to a long half-life, lenvatinib can be interrupted to assess whether an AE improves/resolves with dechallenge (i.e., interruption of treatment) based on the following 2 scenarios:
   If an AE is identified during a treatment cycle (i.e., between two Composition A doses), only lenvatinib dose interruption is needed.
   If an AE is identified at the beginning of a treatment cycle, lenvatinib can be interrupted and dosing of Composition A should be held.
If the participant recovers from an AE in response to lenvatinib interruption (i.e., positive dechallenge), the event is more likely to be related to lenvatinib. Otherwise, after excluding other alternative explanations, an immune-related AE should be considered.
2. Severity of AE
If an AE is suspected to be treatment-related and is severe/life-threatening at the time of onset or is rapidly worsened, action including interrupting both drugs and initiating treatment with a corticosteroid (with exception of hypothyroidism, type I diabetes mellitus (TIDM)) and other supportive care should be taken promptly.
3. Participants receiving the combination therapy (Composition A+lenvatinib) must discontinue study therapy if any of the criteria outlined in Section Discontinuation of Study Intervention are below are met.

### Composition a Co-Formulation Administration

Composition A co-formulation will be administered using a 30-minute IV infusion every 3 weeks. Sites should make every effort to target infusion timing to be as close to 30 minutes as possible. However, given the variability of infusion pumps from site to site, a window of −5 minutes and +10 minutes is permitted (i.e., infusion time is 25 to 40 minutes).

### Lenvatinib Administration

On C1D1 and C2D1, lenvatinib will be administered after Composition A administration. Lenvatinib capsules are to be taken orally once daily at approximately the same time without regard to food intake from C1D1 onwards. Participants should not take lenvatinib on PK sampling days before their appointment (i.e., C1D1, C1D15, and C2D1). On C1D1, C1D15, and C2D1, participants should be instructed to bring their lenvatinib to the clinic. Lenvatinib should be administered at the study site at approximately the same time of day to accommodate Pharmacokinetic(s) (PK) sample collection timing.

If a participant misses a dose, it may be taken within 12 hours after the usual time of the dose. If more than 12 hours have elapsed from the time of the usual daily dose, lenvatinib should be taken the next day at the usual time. In the event a participant vomits after lenvatinib administration, the participant should not take another dose until the next scheduled dose.

### Efficacy Endpoint

This study will use objective response rate as the primary efficacy endpoint. Objective response rate is defined as the proportion of participants who have best response as CR or PR. Responses are based on blinded independent central review (BICR) using RECIST 1.1 (modified to follow a maximum of 10 target lesions and a maximum of 5 target lesions per organ). Objective response rate is an appropriate endpoint to evaluate the antitumor activity of investigational treatment arms. For the randomized cohort, ORR is defined as the percentage of participants who achieve a confirmed CR or PR per RECIST 1.1 as assessed by BICR. For the single-arm cohorts, ORR is defined as the percentage of participants who achieve a confirmed CR or PR per RECIST 1.1 as assessed by investigator. For the randomized cohort, PFS is defined as the time from randomization to the first documented disease progression per RECIST 1.1 by BICR or death due to any cause, whichever occurs first.

This study will use PFS as a secondary efficacy endpoint. Progression-free survival is defined as the time from date of randomization/allocation until the first date of disease progression or death from any cause, whichever comes first, based on RECIST 1.1 criteria as assessed by BICR. For the single-arm cohorts, PFS is defined as the time from the first dose of study intervention to the first documented disease progression per RECIST 1.1 by investigator or death due to any cause, whichever occurs first.

Additionally, this study will use OS as an exploratory efficacy endpoint. Overall survival in this study is defined as the time from date of randomization/allocation to death from any cause. For the randomized cohort, OS is defined as the time from randomization to death due to any cause. For the single-arm cohorts, OS is defined as the time from the first dose of study intervention to death due to any cause.

### Inclusion Criteria

All participants will be given a participant identification card identifying them as participants in a research study. The card will contain study-site contact information (including direct telephone numbers) to be used in the event of an emergency. The investigator or qualified designee will provide the participant with a participant identification card immediately after the participant provides documented informed consent. At the time of intervention allocation/randomization, site personnel will add the treatment/randomization number to the participant identification card.

The participant ID card also contains contact information for the emergency unblinding call center so that a health care provider can obtain information about study intervention in emergency situations where the investigator is not available.

A medical history will be obtained by the investigator or qualified designee. The medical history will collect all active conditions and any condition diagnosed within the prior 10 years that the investigator considers to be clinically important. Details regarding the disease for which the participant has enrolled in this study will be recorded separately and not listed as medical history.

If a medical condition is diagnosed at the time of Screening due to the physical examination, laboratory tests, radiologic assessment, other assessment, and/or a combination of these evaluations, the medical condition is to be recorded as a baseline condition along with the participant's other medical history unless due to any protocol-specified intervention (e.g., procedure, washout or run-in treatment including placebo run-in).

Comprehensive details regarding the participant's cancer history will be recorded separately and not listed as medical history. These details include, but are not limited to, stage at diagnosis, histopathology, location(s) of tumor burden, investigator-determined tumor size per RECIST 1.1, and all prior treatment (including prior radiation, prior chemotherapy, and prior surgery).

The investigator or qualified designee will review before medication use, including any protocol-specified washout requirement, and record prior medication taken by the participant within 28 days before the first dose of study intervention.

The investigator or qualified designee will record medication, if any, taken by the participant during the study.

All consented participants will be given a unique screening number that will be used to identify the participant for all procedures that occur before randomization/intervention allocation. Each participant will be assigned only 1 screening number. Screening numbers must not be reused for different participants.

Any participant who is screened multiple times will retain the original screening number assigned at the initial Screening Visit.

All eligible participants will be allocated, by nonrandom assignment, and will receive a treatment/randomization number. The treatment/randomization number identifies the participant for all procedures occurring after treatment allocation/randomization. Once a treatment/randomization number is assigned to a participant, it can never be reassigned to another participant.

A single participant cannot be assigned more than 1 treatment/randomization number.

### Exclusion Criteria

The participant must be excluded from the study if the participant:
1. Has a history of a second malignancy, unless potentially curative treatment has been completed with no evidence of malignancy for 3 years. Note: The time requirement does not apply to participants who underwent successful definitive resection of basal cell carcinoma of the skin, squamous cell carcinoma of the skin, in-situ cervical cancer, or other in-situ cancers.
2. Human immunodeficiency virus (HIV)-infected participants with a history of Kaposi's sarcoma and/or Multicentric Castleman's Disease.
3. Has received prior therapy with an anti-PD-1, anti-PD-L1, anti-PD-L2, or anti-TIGIT agent.
4. Has received prior systemic anticancer therapy including investigational agents within 4 weeks before randomization/allocation. Note: Participants must have recovered from all AEs due to previous therapies to ≤Grade 1 or baseline. Participants with ≤Grade 2 neuropathy may be eligible. Participants with endocrine-related AEs Grade ≤2 requiring treatment or hormone replacement may be eligible. Participants with Grade ≤2 alopecia are eligible. Note: If the participant had a major operation, the participant must have recovered adequately from the procedure and/or any complications from the operation before starting study intervention.
5. Has received prior radiotherapy within 2 weeks of start of study intervention. Participants must have recovered from all radiation-related toxicities, not require corticosteroids, and not have had radiation pneumonitis. A 1-week washout is permitted for palliative radiation (≤2 weeks of radiotherapy) to non-central nervous system (CNS) disease.
6. Has received a live or live-attenuated vaccine within 30 days before the first dose of study intervention. Administration of killed vaccines are allowed.
7. Is currently participating in or has participated in a study of an investigational agent or has used an investigational device within 4 weeks before the first dose of study intervention. Note: Participants who have entered the follow-up phase of an investigational study may participate as long as it has been 4 weeks after the last dose of the previous investigational agent.
8. Has a diagnosis of immunodeficiency or is receiving chronic systemic steroid therapy (dosing exceeding 10 mg daily of prednisone equivalent) or any other form of immunosuppressive therapy within 7 days before the first dose of study medication.
9. Has known active CNS metastases and/or carcinomatous meningitis. Participants with previously treated brain metastases may participate provided they are radiologically stable, (i.e., without evidence of progression) for at least 4 weeks by repeat imaging (Note: The repeat imaging should be performed during study Screening), clinically stable, and without requirement of steroid treatment for at least 14 days before the first dose of study intervention. Note: Participants with known untreated, asymptomatic brain metastases (i.e., no neurological symptoms, no requirement for corticosteroids, no or minimal surrounding edema, and no lesion >1.5 cm) may participate but will require regular imaging of the brain as a site of disease.
10. Known severe hypersensitivity (≥Grade 3) to Composition A, pembrolizumab, lenvatinib and/or any of their excipients.
11. Has an active autoimmune disease that has required systemic treatment in past 2 years (i.e., with use of disease modifying agents, corticosteroids or immunosuppressive drugs). Replacement therapy (e.g., thyroxine, insulin, or physiologic corticosteroid replacement therapy for adrenal or pituitary insufficiency) is not considered a form of systemic treatment and is allowed.
12. Has a history of (noninfectious) pneumonitis/interstitial lung disease that required steroids or has current pneumonitis/interstitial lung disease.
13. Has an active infection requiring systemic therapy.
14. Has a history or current evidence of any condition, therapy, or laboratory abnormality that might confound the results of the study, interfere with the participant's participation for the full duration of the study, or is not in the best interest of the participant to participate, in the opinion of the treating investigator.
15. Has a known psychiatric or substance abuse disorder that would interfere with the participant's ability to cooperate with the requirements of the study.
16. Has present or progressive accumulation of pleural, ascitic, or pericardial fluid requiring drainage or diuretic drugs within 2 weeks before randomization/allocation.
17. Has concurrent active Hepatitis B (defined as hepatitis B surface antigen (HBsAg) positive and/or detectable hepatitis B virus (HBV) DNA) and Hepatitis C virus (defined as anti-hepatitis C virus (HCV) Ab positive and detectable HCV RNA) infection. Note: Hepatitis B and C screening tests are not required unless known history of HBV and HCV infection or As mandated by local health
18. Participant, in the judgment of the investigator, is unlikely to comply with the study procedures, restrictions, and requirements of the study.
19. Has had an allogenic tissue/solid organ transplant.
20. Has received previous treatment with lenvatinib. Note: Prior therapy with other kinase inhibitors that target vascular endothelial growth factor (VEGF) are not exclusionary.
21. Has had major surgery within 3 weeks before first dose of study interventions. Note: Adequate wound healing after major surgery must be assessed clinically, independent of time elapsed for eligibility
22. Has preexisting ≥Grade 3 gastrointestinal or non-gastrointestinal fistula.
23. Has urine protein ≥1 g/24 hours. Note: Participants with proteinuria ≥2+(≥100 mg/dL) on urine dipstick testing (urinalysis) will undergo 24-hour urine collection for quantitative assessment of proteinuria.
24. Has a left ventricular ejection fraction (LVEF) below the institutional (or local laboratory) normal range, as determined by multiple-gated acquisition (MUGA) or echocardiogram (ECHO).
25. Has radiographic evidence of encasement or invasion of a major blood vessel, or of intratumoral cavitation. The degree of proximity to major blood vessels should be considered because of the potential risk of severe hemorrhage associated with tumor shrinkage/necrosis after lenvatinib therapy.
26. Has prolongation of corrected QT interval by Fridericia (QTcF) interval to >480 ms. If the QTcF is prolonged to >480 ms in the presence of a pacemaker, contact the Sponsor to determine eligibility
27. Has clinically significant cardiovascular disease within 12 months from first dose of study intervention, including New York Heart Association (NYHA) Class III or IV congestive heart failure, unstable angina, myocardial infarction, cerebral vascular accident, or cardiac arrhythmia associated with hemodynamic instability. Note: Medically controlled arrhythmia would be permitted.
28. Has serious nonhealing wound, ulcer, or bone fracture.
29. Has gastrointestinal (GI) malabsorption or any other condition that might affect the absorption of lenvatinib.
30. Has active hemoptysis (bright red blood of at least 0.5 teaspoon) within 3 weeks before the first dose of study intervention.
31. Has a history of arterial thromboembolism within 12 months of start of study intervention.

### Dose Limiting Toxicity

All toxicities will be graded using National Cancer Institute (NCI) CTCAE Version 5.0 based on the investigator assessment. The DLT window of observation will be during Cycle 1 for each dose level.

The occurrence of any of the following toxicities during Cycle 1 of each dose level will be considered a DLT, if assessed by the investigator to be related to study intervention administration:
a. Grade 4 nonhematologic toxicity (not laboratory).
b. Grade 4 hematologic toxicity lasting ≥7 days, except thrombocytopenia:
   Grade 4 thrombocytopenia of any duration
   Grade 3 thrombocytopenia associated with clinically significant bleeding
c. Any nonhematologic AE≥Grade 3 in severity should be considered a DLT, with the following exceptions: Grade 3 fatigue lasting ≤3 days; Grade 3 diarrhea, nausea, or vomiting without use of antiemetics or antidiarrheals per standard of care; Grade 3 rash without use of corticosteroids or anti-inflammatory agents per standard of care. Exception: This excludes Grade 3 hypertension able to be controlled by medication for those participants receiving lenvatinib
d. Any Grade 3 or Grade 4 nonhematologic laboratory value if:
   Clinically significant medical intervention is required to treat the participant, or
   The abnormality leads to hospitalization, or
   The abnormality persists for >1 week
   The abnormality results in a drug-induced liver injury (DILI)
Exceptions: Clinically nonsignificant, treatable, or reversible laboratory abnormalities including liver function tests, uric acid, etc.
e. Febrile neutropenia Grade 3 or Grade 4:
   Grade 3 is defined as absolute neutrophil count (ANC<1000/mm³ with a single temperature of >38.3 degrees C. (101 degrees F.) or a sustained temperature of ≥38 degrees C. (100.4 degrees F.) for more than 1 hour.
   Grade 4 is defined as ANC<1000/mm³ with a single temperature of >38.3 degrees C. (101 degrees F.) or a sustained temperature of ≥38 degrees C. (100.4 degrees F.) for more than 1 hour, with life-threatening consequences and urgent intervention indicated.
f. Prolonged delay (>2 weeks) in initiating Cycle 2 due to intervention-related toxicity.
g. Any intervention-related toxicity that causes the participant to discontinue intervention during Cycle 1.
h. Grade 5 toxicity.

### Assessment at Screening and Prior to RECIST 1.1 Progression

Until radiographic disease progression based on RECIST 1.1, there is no distinct iRECIST assessment.

### Assessment and Decision at RECIST 1.1 Progression

For participants who show radiological disease progression by RECIST 1.1, the investigator will decide whether to continue a participant on study intervention until repeat scans 4 to 8 weeks later are obtained.

Tumor flare may manifest as any factor causing radiographic progression per RECIST 1.1, including:
Increase in the sum of diameters of target lesion(s) identified at baseline to ≥20% and ≥5 mm from nadir. The Response Evaluation Criteria in Solid Tumors 1.1 for immune-based therapeutics (iRECIST) publication uses the terminology “sum of measurements,” but “sum of diameters” will be used in this protocol, consistent with the original RECIST 1.1 terminology.
Unequivocal progression of nontarget lesion(s) identified at baseline
Development of new lesion(s)

iRECIST defines new response categories, including iUPD (unconfirmed progressive disease) and iCPD (confirmed progressive disease). For purposes of iRECIST assessment, the first visit showing progression according to RECIST 1.1 will be assigned a visit (overall) response of iUPD, regardless of which factors caused the progression.

At this visit, target and nontarget lesions identified at baseline by RECIST 1.1 will be assessed as usual.

New lesions will be classified as measurable or nonmeasurable, using the same size thresholds and rules as for baseline lesion assessment in RECIST 1.1. From measurable new lesions, up to 5 lesions total (up to 2 per organ), may be selected as New Lesions—Target. The sum of diameters of these lesions will be calculated and kept distinct from the sum of diameters for target lesions at baseline. All other new lesions will be followed qualitatively as New Lesions—Nontarget.

### Assessment at the Confirmatory Imaging

On the confirmatory scans, the participant will be classified as progression confirmed (with an overall response of iCPD), or as showing persistent unconfirmed progression (with an overall response of iUPD), or as showing disease stability or response (stable disease by iRECIST (iSD)/partial response by iRECIST (iPR)/complete response by iRECIST (iCR)).

### Confirmation of Progression

Progression is considered confirmed, and the overall response will be iCPD, if ANY of the following occurs:
Any of the factors that were the basis for the iUPD at the previous visit show worsening
   For target lesions, worsening is a further increase in the sum of diameters of ≥5 mm, compared with any prior iUPD time point
   For nontarget lesions, worsening is any significant growth in lesions overall, compared with a prior iUPD time point; this does not have to meet the “unequivocal” standard of RECIST 1.1
   For new lesions, worsening is any of these:
      An increase in the new lesion sum of diameters by ≥5 mm from a prior iUPD time point
      Visible growth of new nontarget lesions
      The appearance of additional new lesions
Any new factor appears that would have triggered disease progression by RECIST 1.1
   Persistent iUPD

Progression is considered not confirmed, and the overall response remains iUPD, if:
None of the progression-confirming factors identified above occurs AND
The target lesion sum of diameters (initial target lesions) remains above the initial disease progression threshold (by RECIST 1.1)

Additional scans for confirmation are to be scheduled 4 to 8 weeks from the scans on which iUPD is seen. This may correspond to the next visit in the original visit schedule. The assessment of the subsequent confirmation scan proceeds in an identical manner, with possible outcomes of iCPD, iUPD, and iSD/iPR/iCR.

Resolution of iUPD

Progression is considered not confirmed, and the overall response becomes iSD/iPR/iCR, if:
None of the progression-confirming factors identified above occurs, AND
The target lesion sum of diameters (initial target lesions) is not above the disease progression threshold.

The response is classified as iSD or iPR (depending on the sum of diameters of the target lesions), or iCR if all lesions resolve.

In this case, the initial iUPD is considered to be pseudoprogression, and the level of suspicion for progression is “reset.” This means that the next visit that shows radiographic progression, whenever it occurs, is again classified as iUPD by iRECIST, and the confirmation process is repeated before a response of iCPD can be assigned.

### Management Following the Confirmatory Imaging

If repeat scans do not confirm disease progression, and the participant continues to be clinically stable, study intervention is to continue. The regular scan schedule is to be followed. If disease progression is confirmed, participants may be discontinued from study intervention.

If a participant has confirmed radiographic progression (iCPD) and clinically meaningful benefit, study intervention may be continued after consultation with the Sponsor. If study intervention is continued, tumor scans are to be performed after the intervals.

Detection of Progression at Visits after Pseudo-Progression Resolves

After resolution of pseudoprogression (i.e., after iSD/iPR/iCR), another instance of progression (another iUPD) is indicated by any of the following events:
Target lesions
   Sum of diameters reaches the disease progression threshold (≥20% and ≥5 mm increase from nadir) either for the first time, or after resolution of previous pseudoprogression. The nadir is always the smallest sum of diameters seen during the entire study, either before or after an instance of pseudoprogression.
Nontarget lesions
   If nontarget lesions have never shown unequivocal progression, their doing so for the first-time results in iUPD.
   If nontarget lesions have shown previous unequivocal progression, and this progression has not resolved, iUPD results from any significant further growth of nontarget lesions, taken as a whole.
New lesions
   New lesions appear for the first time
   Additional new lesions appear
   Previously identified new target lesions show an increase of ≥5 mm in the new lesion sum of diameters, from the nadir value of that sum
   Previously identified nontarget lesions show any significant growth

If any of the events above occur, the overall response for that visit is iUPD, and the iUPD evaluation process (see Assessment at the Confirmatory Scan above) is repeated. Progression must be confirmed before iCPD can occur.

The decision process on the subsequent iUPD is identical to the iUPD confirmation process for the initial disease progression, with one exception, which can occur if new lesions had occurred at a prior instance of iUPD, had not resolved, then worsened (increase in size or number) leading to the second iUPD. If new lesion worsening has not resolved at the confirmatory scan, then iUPD cannot resolve to iSD or iPR. It will remain iUPD until either a decrease in the new lesion burden allows resolution to iSD or iPR, or until new or worsening cause of progression indicates iCPD.

### Discontinuation

Discontinuation of study intervention does not represent withdrawal from the study. As certain data on clinical events beyond study intervention discontinuation may be important to the study, they must be collected through the participant's last scheduled follow-up, even if the participant has discontinued study intervention. Therefore, all participants who discontinue study intervention before completion of the protocol-specified treatment period will still continue to be monitored in the study and participate in the study visits and procedures unless the participant has withdrawn from the study.

Participants may discontinue study intervention at any time for any reason or be discontinued from the study intervention at the discretion of the investigator should any untoward effect occur. In addition, a participant may be discontinued from study intervention by the investigator or the Sponsor if study intervention is inappropriate, the study plan is violated, or for administrative and/or other safety reasons.

A participant must be discontinued from study intervention, but continue to be monitored in the study for any of the following reasons:
The participant or participant's legally acceptable representative requests to discontinue study intervention.
Any prolonged interruption of study intervention beyond the permitted periods, for irAE or AE management or other allowed dose interruptions, require Sponsor consultation before restarting treatment. If treatment will not be restarted, the participant will continue to be monitored in the study and the reason for discontinuation of study intervention will be recorded in the medical record.
The participant has a medical condition or personal circumstance, which in the opinion of the investigator and/or Sponsor, placed the participant at unnecessary risk from continued administration of study intervention.
The participant has a confirmed positive serum pregnancy test.
Radiographic disease progression (after obtaining informed consent addendum and Sponsor communication, the investigator may elect to continue treatment beyond imaging CRO (iCRO)-verified disease progression).
Any progression or recurrence of malignancy, or any occurrence of another malignancy that requires active treatment.
Any study intervention-related toxicity specified as a reason for permanent discontinuation as defined in the guidelines for dose modification due to AEs.
The participant requires any of the prohibited concomitant medication listed
Participants receiving the combination of Composition A+Lenvatinib for endometrial cancer must discontinue study therapy if any of the following occur:
   Alanine aminotransferase (ALT) or aspartate aminotransferase (AST) elevation meeting the following criteria:
      ALT or AST>5×upper limit of normal (ULN) for more than 2 weeks Composition A will have already been permanently discontinued, but lenvatinib may be administered at a reduced dose by the time this criterion is met and must be permanently discontinued immediately.
      ALT or AST>3×ULN and (total bilirubin (TBL)>2×ULN or international normalized ratio (INR)>1.5)
Participants receiving the combination of Composition A+lenvatinib in HCC must discontinue study therapy if any of the following nonoverdose hepatic events of clinical interests (HECIs) occur:
   ALT>20×ULN (confirmed within 1 week).
   Drug-related total bilirubin >10×ULN.
   Child-Pugh score of >9 points if not improved to Child-Pugh score <9 by intervention within 3 days.
   Hepatic encephalopathy not manageable by therapy within 3 days.
   Clinical ascites not manageable by therapy within 3 days.
Recurrence of a severe or life-threatening event, or of any of the laboratory abnormalities listed above, that are presumed to be immune-related.

A participant must be withdrawn from the study if the participant or participant's legally acceptable representative withdraws consent from the study. If a participant withdraws from the study, they will no longer receive study intervention or be followed at scheduled protocol visits. If a participant fails to return to the clinic for a required study visit and/or if the site is unable to contact the participant, the following procedures are to be performed:
The site must attempt to contact the participant and reschedule the missed visit. If the participant is contacted, the participant should be counseled on the importance of maintaining the protocol-specified visit schedule.
The investigator or designee must make every effort to regain contact with the participant at each missed visit (e.g., telephone calls and/or a certified letter to the participant's last known mailing address or locally equivalent methods). These contact attempts should be documented in the participant's medical record.
Note: A participant is not considered lost to follow-up until the last scheduled visit for the individual participant. The missing data for the participant will be managed via the prespecified statistical data handling and analysis guidelines.

### Informed Consent

The investigator or medically qualified designee (consistent with local requirements) must obtain documented informed consent from each potential participant (or their legally acceptable representative) prior to participating in this clinical study If there are changes to the participant's status during the study (e.g., health or age of majority requirements), the investigator or medically qualified designee must ensure the appropriate documented informed consent is in place.

### Tumor Imaging and Assessment of Disease

The term “scan” refers to any medical imaging data used to assess tumor burden and may include cross-sectional imaging (such as CT or MRI), medical photography, or other methods as specified in this protocol.

In addition to survival, efficacy will be assessed based on evaluation of scan changes in tumor burden over time, until the participant is discontinued from the study or goes into survival follow-up. The process for scan collection and transmission to the iCRO can be found in the Site Imaging Manual (SIM). Tumor scans by CT are strongly preferred. For the abdomen and pelvis, contrast-enhanced MRI may be used when CT with iodinated contrast is contraindicated, or when mandated by local practice. For participants with HCC, triple phase imaging of the liver is required, as described in the SIM. The same scan technique should be used in a participant throughout the study to optimize the reproducibility of the assessment of existing and new tumor burden and improve the accuracy of the response assessment based on scans.

For the purposes of assessing tumor scans, the term “investigator” refers to the local investigator at the site and/or the radiological reviewer at the site or at an offsite facility.

If brain scans are performed, MRI is preferred; however, CT imaging will be acceptable, if MRI is medically contraindicated.

Bone scans may be performed to evaluate bone metastases. Any supplemental scans performed to support a positive or negative bone scan, such as plain X-rays acquired for correlation, should also be submitted to the iCRO.

Other imaging modalities that may be collected, submitted to the iCRO, and included in the response assessment and those that should not be submitted to the iCRO and will not be included in response assessment are defined in the SIM.

At Screening, participant eligibility will require radiographic documentation of at least 1 lesion that meets the requirements for selection as a target lesion, before participant allocation.

All scheduled scans for participants will be submitted to the iCRO. In addition, a scan that is obtained at an unscheduled time point, for any reason (including suspicion of progression or other clinical reason), should also be submitted to the iCRO if it shows disease progression, or if it is used to support a response assessment. All scans acquired within the protocol-specified window of time around a scheduled scan visit are to be classified as pertaining to that visit.

### Initial Tumor Imaging

Initial tumor scans at Screening must be performed within 28 days before the date of randomization/allocation. Any scans obtained after Cycle 1 Day 1 cannot be included in the Screening assessment. The site must review Screening scans to confirm the participant has measurable disease per RECIST 1.1.

Tumor scans performed as part of routine clinical management are acceptable for screening if they are of acceptable diagnostic quality and performed within 28 days of randomization and can be assessed by the iCRO.

If brain scans are required to document the stability of existing metastases, the brain MRI should be acquired during Screening.

Bone scans are required at Screening for participants with a history of bone metastases and/or for those participants with indicative clinical signs/symptoms such as bone pain or elevated alkaline phosphatase levels.

### Tumor Imaging During the Study

The first on study scan should be performed at 9 weeks (63 days+7 days) from the date of randomization/allocation. Subsequent tumor scans should be performed every 9 weeks (63 days±7 days) or more frequently if clinically indicated. After 54 weeks (378 days±7 days), participants who remain on treatment will have scans performed every 12 weeks (84 days±7 days). Scan timing should follow calendar days and should not be adjusted for delays in cycle starts. Scans are to be performed until disease progression is identified by the investigator or notification by the Sponsor, or until the start of new anticancer treatment, withdrawal of consent, or death, whichever occurs first.

Or should be confirmed by a repeat scan performed at least 4 weeks after the first indication of a response is observed. Participants will then return to the regular scan schedule, starting with the next scheduled time point. Participants who receive additional scans for confirmation do not need to undergo the next scheduled scan if it is fewer than 4 weeks later; scans may resume at the subsequent scheduled time point.

On study brain or bone scans should be performed if clinically indicated or to confirm CR (if other lesions indicate CR and brain or bone lesions existed at baseline).

When radiological disease progression is identified by the investigator in clinically stable participants, disease progression is to be confirmed by another set of scans performed 4 to 8 weeks later.

If disease progression is not confirmed, clinically stable participants are to continue study intervention until progression is confirmed. Participants are to return to their regular scan schedule. If the next scheduled scan will occur in less than 4 weeks, this scheduled scan may be skipped.

If disease progression is confirmed, study intervention will be discontinued.

### Safety Endpoints

The safety endpoints include AEs, SAEs, and study intervention discontinuation due to AEs. Safety and tolerability will be assessed by clinical review of all relevant parameters including AEs, laboratory tests and vital signs. For cohorts with the safety lead-in phase, the safety endpoints also include the incidence of DLTs.

### Adverse Event (AE)

An AE is any untoward medical occurrence in a clinical study participant, temporally associated with the use of study intervention, whether or not considered related to the study intervention.

An AE can therefore be any unfavorable and unintended sign (including an abnormal laboratory finding), symptom, or disease (new or exacerbated) temporally associated with the use of a study intervention.

For purposes of AE definition, study intervention (also referred to as Sponsor's product) includes any pharmaceutical product, biological product, vaccine, diagnostic agent, medical device, combination product, or protocol specified procedure whether investigational or marketed (including placebo, active comparator product, or run-in intervention), manufactured by, licensed by, provided by, or distributed by the Sponsor for human use in this study. The following are included as AEs:
Any abnormal laboratory test results (hematology, clinical chemistry, or urinalysis) or other safety assessments (e.g., electrocardiogram (ECG), radiological scans, vital signs measurements), including those that worsen from baseline, considered clinically significant in the medical and scientific judgment of the investigator.
Exacerbation of a chronic or intermittent preexisting condition including either an increase in frequency and/or intensity of the condition.
New conditions detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.
For all reports of overdose (whether accidental or intentional) with an associated AE, the AE term should reflect the clinical symptoms or abnormal test result. An overdose without any associated clinical symptoms or abnormal laboratory results is reported using the terminology “accidental or intentional overdose without adverse effect.”
Any new cancer (that is not a condition of the study). Progression of the cancer under study is not a reportable event.

The following events do not meet the AE definition for the purpose of this study:
Medical or surgical procedure (e.g., endoscopy, appendectomy): the condition that leads to the procedure is the AE.
Situations in which an untoward medical occurrence did not occur (social and/or convenience admission to a hospital).
Anticipated day-to-day fluctuations of preexisting disease(s) or condition(s) present or detected at the start of the study that do not worsen.
Surgical procedure(s) planned prior to informed consent to treat a preexisting condition that has not worsened.

### Serious Adverse Event (SAE)

If an event is not an AE per the above, then it cannot be an SAE even if serious conditions are met. An SAE is defined as any untoward medical occurrence that, at any dose:
Results in death.
Is life-threatening. The term “life-threatening” in the definition of “serious” refers to an event in which the participant was at risk of death at the time of the event. It does not refer to an event, which hypothetically might have caused death, if it were more severe.
Requires inpatient hospitalization or prolongation of existing hospitalization. Hospitalization is defined as an inpatient admission, regardless of length of stay, even if the hospitalization is a precautionary measure for continued observation. (Note: Hospitalization for an elective procedure to treat a pre-existing condition that has not worsened is not an SAE. A pre-existing condition is a clinical condition that is diagnosed prior to the use of a product and is documented in the participant's medical history.
Results in persistent or significant disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.
Is a congenital anomaly/birth defect. In offspring of participant taking the product regardless of time to diagnosis.
Other important medical events. Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

The study treatments are outlined below in Table 14 and Table 15.

TABLE 14
Investigation summary - initial treatment

| Initial Treatment/First Couse | | | | | | | | | Post-treatment Visits | | | |
|---|---|---|---|---|---|---|---|---|---|---|---|---|
| | Study Period | | | | | | | | | | | |
| | | | | | | | | | Safety | Efficacy | Survival | |
| | Screening | Treatment Cycle = 21 Days | | | | | | EOT | FU | FU | FU | |
| | Visit Timing/Cycle Number | | | | | | | | | | | |
| | | | | | | | | | 30 | | | |
| | | | | | | | | | Days | | | |
| | | | | | | | | | post | Q9W | | |
| | | | | | | | 6 to | At | last | or | | Notes |
| | −28 to −1 | 1 | 2 | 3 | 4 | 5 | 35 | DC | dose | Q12W | Q12W | All procedures are to be |

| | |
|---|---|
| Cycle Day | performed before study |

| | | | | | | | |
|---|---|---|---|---|---|---|---|
| 1 | 1 | 1 | 1 | 1 | 1 | — | intervention |

| | |
|---|---|
| Scheduling Window (Days) | administration unless |

| | | | | | | | | | |
|---|---|---|---|---|---|---|---|---|---|
| ±3 | ±3 | ±3 | ±3 | ±3 | ±3 | ±7 | ±7 | ±14 | otherwise indicated. |

Administrative Procedures

| | | | | | | | | | | | | |
|---|---|---|---|---|---|---|---|---|---|---|---|---|
| Informed consent | X | | | | | | | | | | | Documented informed |
| | | | | | | | | | | | | consent must be obtained |
| | | | | | | | | | | | | before any protocol- |
| | | | | | | | | | | | | specific screening |
| | | | | | | | | | | | | procedures are |
| | | | | | | | | | | | | performed. |
| | | | | | | | | | | | | If the investigator plans |
| | | | | | | | | | | | | to treat beyond disease |
| | | | | | | | | | | | | progression, additional |
| | | | | | | | | | | | | consent is required. |
| Inclusion/exclusion | X | | | | | | | | | | | |
| criteria | | | | | | | | | | | | |
| Participant | X | X | | | | | | | | | | Update with |
| identification card | | | | | | | | | | | | randomization/allocation |
| | | | | | | | | | | | | number at C1D1. |
| Demographic | X | | | | | | | | | | | |
| Medical history | X | | | | | | | | | | | |
| Cancer history | X | | | | | | | | | | | Review prior surgery, |
| | | | | | | | | | | | | radiation, and oncologic |
| | | | | | | | | | | | | therapy |
| FIGO stage at diagnosis | X | | | | | | | | | | | |
| Prior and concomitant | X | X | X | X | X | X | X | X | X | | | Prior concomitant |
| medications review | | | | | | | | | | | | medications received |
| | | | | | | | | | | | | within 28 days before the |
| | | | | | | | | | | | | first dose of study |
| | | | | | | | | | | | | intervention through |
| | | | | | | | | | | | | 30 days after the last dose |
| | | | | | | | | | | | | of study intervention. |
| | | | | | | | | | | | | All concomitant |
| | | | | | | | | | | | | medications administered |
| | | | | | | | | | | | | during SAEs or ECIs are |
| | | | | | | | | | | | | to be recorded. |
| Treatment | | X | | | | | | | | | | Dose within 3 days of |
| randomization/allocation | | | | | | | | | | | | randomization/allocation. |
| Subsequent | | | | | | | | | X | X | X | |
| antineoplastic treatment | | | | | | | | | | | | |
| Survival status | | | | X | | | | X | X | X | X | Updated survival status |
| | | | | | | | | | | | | may be requested by the |
| | | | | | | | | | | | | Sponsor at any time |
| | | | | | | | | | | | | during the course of the |
| | | | | | | | | | | | | study. |

Interventions

| | | | | | | | |
|---|---|---|---|---|---|---|---|
| Composition A | X | X | X | X | X | X | Co-formulation: 200 mg |
| | | | | | | | vibostolimab + 200 mg |
| | | | | | | | pembrolizumab Q3W |

Efficacy Assessments

| Brain imaging | X | X | | | Only required at |
|---|---|---|---|---|---|
| (MRI; CT imaging will | | | | | Screening for participants |
| be acceptable if MRI is | | | | | with brain metastases at |
| contraindicated) | | | | | (to demonstrate stability) |
| | | | | | or who are clinically |
| | | | | | symptomatic. |
| | | | | | On study imaging as |
| | | | | | clinically indicated. |
| | | | | | Perform at CR to confirm |
| | | | | | response for participants |
| | | | | | with brain metastases at |
| | | | | | baseline or at any other |
| | | | | | point during the study. |
| Bone imaging | X | X | | | Only required at |
| | | | | | Screening for participants |
| | | | | | with a history of bone |
| | | | | | metastases or who are |
| | | | | | clinically symptomatic. |
| | | | | | On study imaging as |
| | | | | | clinically indicated. |
| | | | | | Perform at CR to confirm |
| | | | | | response for participants |
| | | | | | with bone metastases at |
| | | | | | baseline or at any other |
| | | | | | point during the study. |
| Tumor imaging and | X | X | X | X | Schedule should be |
| response assessment | | | | | followed regardless of |
| (chest, abdomen, and | | | | | treatment delays. |
| pelvis) | | | | | If imaging was obtained |
| | | | | | within 4 weeks before |
| | | | | | treatment DC, scan at DC |
| | | | | | is not mandatory. Follow- |
| | | | | | up visits may be |
| | | | | | scheduled to coincide |
| | | | | | with the imaging |
| | | | | | schedule. |

Clinical Procedures/Assesments

| | | | | | | | | | | | |
|---|---|---|---|---|---|---|---|---|---|---|---|
| AE/SAE monitoring | X | X | X | X | X | X | X | X | X | X | Report AEs occurring |
| | | | | | | | | | | | within 30 days after the |
| | | | | | | | | | | | last dose of study |
| | | | | | | | | | | | intervention. |
| | | | | | | | | | | | Report SAEs occurring |
| | | | | | | | | | | | within 90 days after the |
| | | | | | | | | | | | last dose of study |
| | | | | | | | | | | | intervention, or 30 days |
| | | | | | | | | | | | after the last dose of |
| | | | | | | | | | | | study intervention if a |
| | | | | | | | | | | | new anticancer therapy is |
| | | | | | | | | | | | initiated, whichever is |
| | | | | | | | | | | | earlier. |
| Complete physical | X | | | | | | | X | | | To be performed within 7 |
| examination | | | | | | | | | | | days before start of study |
| | | | | | | | | | | | intervention. |
| Directed physical | | X | X | X | X | X | X | | X | | |
| examination | | | | | | | | | | | |
| Height | X | | | | | | | | | | |
| Vital sign | X | X | X | X | X | X | X | X | X | | Predose and as clinically |
| measurements, weight | | | | | | | | | | | indicated. |
| 12-lead ECG | X | | | | | | | X | X | | |
| ECOG performance | X | X | X | X | X | X | X | X | X | | Performed within 7 days |
| status | | | | | | | | | | | before starting study |
| | | | | | | | | | | | intervention and before |
| | | | | | | | | | | | each treatment |
| | | | | | | | | | | | administration. |

Laboratory Assessments - Local Laboratory

| | | | | | | | | | |
|---|---|---|---|---|---|---|---|---|---|
| Pregnancy test for | X | | | | | | X | X | WOCBP require negative |
| WOCBP | | | | | | | | | serum test within 72 |
| | | | | | | | | | hours or negative urine |
| | | | | | | | | | test within 24 hours |
| | | | | | | | | | before starting |
| | | | | | | | | | Composition A. |
| | | | | | | | | | Additional pregnancy |
| | | | | | | | | | testing to be conducted as |
| | | | | | | | | | clinically indicated. |
| HBV/HCV/HIV | X | | | | | | | | Testing is only required if |
| | | | | | | | | | mandated by local |
| | | | | | | | | | regulations. |
| CBC with differential | X | X | X | X | X | X | X | X | Performed within 10 days |
| Chemistry Panel | X | X | X | X | X | X | X | X | before first dose. |
| Urinalysis | X | | | | X | X | X | X | After C1, collect within |
| | | | | | | | | | 3 days before dosing. |
| | | | | | | | | | Urinalysis should be |
| | | | | | | | | | performed every 4 cycles |
| | | | | | | | | | (C5, C9, etc.). |
| PT/INR and aPTT/PTT | X | | | | | | | | Screening samples |
| | | | | | | | | | collected within 10 days |
| | | | | | | | | | before first dose of study |
| | | | | | | | | | intervention. |
| | | | | | | | | | Additional testing to be |
| | | | | | | | | | conducted as clinically |
| | | | | | | | | | indicated for participants |
| | | | | | | | | | taking anticoagulant |
| | | | | | | | | | therapy. |
| Thyroid function (T3, | X | X | | X | | X | X | X | Should be performed at |
| T4, and TSH) | | | | | | | | | Screening and predose of |
| | | | | | | | | | every other cycle starting |
| | | | | | | | | | from C2 (i.e., C4, C6, C8 |
| | | | | | | | | | etc.). Participants may be |
| | | | | | | | | | dosed in subsequent |
| | | | | | | | | | cycles after C1 while |
| | | | | | | | | | thyroid function tests are |
| | | | | | | | | | pending. |
| | | | | | | | | | Free T3 and free T4 are |
| | | | | | | | | | acceptable. |

Laboratory Assessments - Central Laboratory

| | | | | | | | | | |
|---|---|---|---|---|---|---|---|---|---|
| PD-L1 status | X | | | | | | | | PD-L1 status can be |
| | | | | | | | | | retrospectively |
| | | | | | | | | | determined. |
| MMR status | X | | | | | | | | Results from the central |
| | | | | | | | | | determination of MMR |
| | | | | | | | | | status are needed before |
| | | | | | | | | | allocation to study |
| | | | | | | | | | treatment. |
| Blood for serum | | X | X | | X | | X* | | PK and ADA samples |
| vibostolimab and | | | | | | | | | should be drawn within |
| pembrolizumab PK | | | | | | | | | 24 hours before study |
| Blood for ADA of | | X | X | | X | | X* | | intervention |
| vibostolimab and | | | | | | | | | administration. |
| pembrolizumab | | | | | | | | | Additional postdose (end |
| | | | | | | | | | of infusion) PK only |
| | | | | | | | | | samples will be drawn |
| | | | | | | | | | within 10 minutes after |
| | | | | | | | | | end of infusion at C1 and |
| | | | | | | | | | C8. |
| | | | | | | | | | *After C4, PK and ADA |
| | | | | | | | | | samples to be collected |
| | | | | | | | | | every 4 cycles (i.e., C8, |
| | | | | | | | | | C12, C16, etc.) through |
| | | | | | | | | | C32. |
| Blood for genetic | | X | | | | | | | Collect before study |
| analysis | | | | | | | | | intervention. |
| Blood for PBMC | | X | X | X | | | | X | Collect at predose on D1 |
| analysis | | | | | | | | | of C1, C2, C3, and at |
| | | | | | | | | | EOT. |
| Blood for ctDNA | | X | X | X | | X | X | X | Collect at predose on D1 |
| Analysis | | | | | | | | | of C1, C2, C3, C5, C7, |
| | | | | | | | | | C10, C13, C16, C19, then |
| | | | | | | | | | on D1 of every 4 cycles |
| | | | | | | | | | (i.e., C23, C27, etc.). |

Tumor Tissue Collection

| | | |
|---|---|---|
| Newly | X | May use archival tissue |
| obtained/archival tissue | | sample obtained before |
| sample for PD-L1 | | Screening as part of the |
| analysis and other | | participant's SOC. |
| biomarkers | | |

| | |
|---|---|
| Patient-reported Outcomes | Perform PRO |
| | assessments in the order |
| | as listed. |

Abbreviations: ADA = antidrug antibody(ies); AE = adverse event; aPTT = activated partial thromboplastin time; BICR = blinded independent central review; BP = blood pressure;; BW = body weight; C# = cycle number; CBC = complete blood count; CR = complete response; CT = computed tomography; ctDNA = circulating tumor DNA; D# = day number; DC = discontinuation; dMMR = mismatch repair deficient; ECG = electrocardiogram; ECHO = echocardiogram; ECI = event(s) of clinical interest; ECOG = Eastern Cooperative Oncology Group; EORTC = European Organisation for Research and Treatment of Cancer; EOT = end-of-treatment; EQ-5D-5L = EuroQol 5-dimension 5-level; FIGO = International Federation of Gynecology and Obstetrics; FISH = fluorescent in-situ hybridization; HBV = hepatitis B virus; HCV = hepatitis C virus; HER2 = human epidermal growth factor receptor 2; HIV = human immunodeficiency virus; HPV = human papilloma virus; HRQoL = health-related quality-of-life; INR = international normalized ratio; IV = intravenous(ly); LVEF = left ventricular ejection fraction; MRI = magnetic resonance imaging; MUGA = multiple-gated acquisition; NYHA = New York Heart Association; PCR = polymerase chain reaction; PD = progressive disease; PD-L1 = programmed cell death 1 ligand 1; PK = pharmacokinetic; pMMR = mismatch repair proficient; PRO = patient-reported outcome; PT = prothrombin time; PTT = partial thromboplastin time; Q3W = every 3 weeks; Q9W = every 9 weeks; Q12W = every 12 weeks; qd = once daily; QLQ-C30 = quality-of-life questionnaire core 30; QLQ-CX24 = cervical cancer-specific quality-of-life questionnaire; SAE = serious adverse event; SOC = standard of care; TNBC = triple-negative breast cancer; T3 = triiodothyronine; T4 = thyroxine; TSH = thyroid stimulating hormone; UPCR = urine protein-to-creatinine ratio; WOCBP = women of childbearing potential.

TABLE 15
Investigation summary - second course treatment

| | | | | | | | | | | | Post-treatment Visits | | | |
|---|---|---|---|---|---|---|---|---|---|---|---|---|---|---|
| | | | | | | | | | | | | | | Notes |
| | | | | | | | | | | | | | | All procedures |
| | | | | | | | | | | | | | | are to be |
| | | | | | | | | | | | | | | performed before |
| Second Course | | | | | | | | | | | | | | study |
| | Study Period | | | | | | | | | | | | | intervention |
| | | | | | | | | | | | Safety | | | administration |
| | Treatment Cycle = 21 Days | | | | | | | | | EOT | FU | | | unless otherwise |
| | Visit Timing/Cycle Number | | | | | | | | | | | | | indicated. All |
| | | | | | | | | | | | 30 | | | lenvatinib- |
| | | | | | | | | | | | Days | | | specific |
| | | | | | | | | | | | post | Efficacy | Survival | procedures are |
| | | | | | | | | | 6 to | At | last | FU | FU | only to be |
| | 1 | | | 2 | | 3 | 4 | 5 | 17 | DC | dose | Q12W | Q12W | performed if |
| | Cycle Day | | | | | | | | | | | | | lenvatinib is |
| | 1 | 8 | 15 | 1 | 15 | 1 | 1 | 1 | 1 | — | | | | administered |
| | Scheduling Window (Days) | | | | | | | | | | | | | during Second |
| | +3 | ±3 | ±3 | ±3 | ±3 | ±3 | ±3 | ±3 | ±3 | | ±7 | ±7 | ±14 | Course. |

Administrative Procedures

| Informed consent | X | | | | | | | | | | | | | |
|---|---|---|---|---|---|---|---|---|---|---|---|---|---|---|
| Inclusion/exclusion | X | | | | | | | | | | | | | |
| criteria | | | | | | | | | | | | | | |
| Prior and | X | X | X | X | X | X | X | X | X | X | X | | | Prior concomitant |
| concomitant | | | | | | | | | | | | | | medications |
| medications review | | | | | | | | | | | | | | received within |
| | | | | | | | | | | | | | | 28 days before the |
| | | | | | | | | | | | | | | first dose of study |
| | | | | | | | | | | | | | | intervention in |
| | | | | | | | | | | | | | | Second Course |
| | | | | | | | | | | | | | | through 30 days |
| | | | | | | | | | | | | | | after the last dose |
| | | | | | | | | | | | | | | of study |
| | | | | | | | | | | | | | | intervention. |
| | | | | | | | | | | | | | | All concomitant |
| | | | | | | | | | | | | | | medications |
| | | | | | | | | | | | | | | administered |
| | | | | | | | | | | | | | | during SAEs or |
| | | | | | | | | | | | | | | ECIs are to be |
| | | | | | | | | | | | | | | recorded. |
| Subsequent | | | | | | | | | | | X | X | X | |
| antineoplastic | | | | | | | | | | | | | | |
| treatment | | | | | | | | | | | | | | |
| Survival status | X | | | | | | | | | X | X | X | X | Participants may |
| | | | | | | | | | | | | | | be contacted for |
| | | | | | | | | | | | | | | survival status at |
| | | | | | | | | | | | | | | any time during |
| | | | | | | | | | | | | | | the study. |
| Home virtual visit | | X | | | | | | | | | | | | Participants will |
| | | | | | | | | | | | | | | be contacted to |
| | | | | | | | | | | | | | | report BP and |
| | | | | | | | | | | | | | | record AEs. Blood |
| | | | | | | | | | | | | | | pressure will be |
| | | | | | | | | | | | | | | taken, for |
| | | | | | | | | | | | | | | example, at home |
| | | | | | | | | | | | | | | or at a local |
| | | | | | | | | | | | | | | pharmacy, and |
| | | | | | | | | | | | | | | will be reviewed |
| | | | | | | | | | | | | | | with the |
| | | | | | | | | | | | | | | investigator or |
| | | | | | | | | | | | | | | designee. |

Interventions

| Composition A | X | | | X | | X | X | X | X | | | | Co-formulation: |
|---|---|---|---|---|---|---|---|---|---|---|---|---|---|
| | | | | | | | | | | | | | 200 mg |
| | | | | | | | | | | | | | vibostolimab + |
| | | | | | | | | | | | | | 200 mg |
| | | | | | | | | | | | | | pembrolizumab |
| | | | | | | | | | | | | | Q3W. |
| Lenvatinib | X | | | | | X | | X | X | X | X | | Continuation of |
| dispensing | | | | | | | | | | | | | lenvatinib is |
| | | | | | | | | | | | | | allowed during |
| | | | | | | | | | | | | | Second Course at |
| | | | | | | | | | | | | | the discretion of |
| | | | | | | | | | | | | | the investigator. |
| | | | | | | | | | | | | | Antineoplastic |
| | | | | | | | | | | | | | therapy different |
| | | | | | | | | | | | | | from lenvatinib is |
| | | | | | | | | | | | | | not permitted. |
| | | | | | | | | | | | | | Lenvatinib to be |
| | | | | | | | | | | | | | self-administered |
| | | | | | | | | | | | | | daily: |
| | | | | | | | | | | | | | 20 mg qd |
| | | | | | | | | | | | | | (Endometrial |
| | | | | | | | | | | | | | cancer) |
| | | | | | | | | | | | | | 12 mg qd (HCC; |
| | | | | | | | | | | | | | BW ≥ 60 kg); |
| | | | | | | | | | | | | | 8 mg qd (HCC; |
| | | | | | | | | | | | | | BW ≤ 60 kg). |
| Lenvatinib bottle | | | | X | | X | X | X | X | X | | | |
| collection | | | | | | | | | | | | | |
| Imaging | | | | | | | | | | | | | Schedule should |
| | | | | | | | | | | | | | be followed |
| | | | | | | | | | | | | | regardless of |
| | | | | | | | | | | | | | treatment delays. |
| Tumor imaging | X | | | | | | | | | X | | X | HCC: imaging |
| and response | | | | | | | | | | | | | must also include |
| assessment (chest, | | | | | | | | | | | | | triphasic abdomen |
| abdomen, and | | | | | | | | | | | | | CT/MRI for liver |
| pelvis) | | | | | | | | | | | | | evaluation. |
| | | | | | | | | | | | | | Images are for |
| | | | | | | | | | | | | | investigator |
| | | | | | | | | | | | | | assessment of |
| | | | | | | | | | | | | | disease status and |
| | | | | | | | | | | | | | are not required |
| | | | | | | | | | | | | | but may be sent to |
| | | | | | | | | | | | | | the iCRO. If |
| | | | | | | | | | | | | | tumor image |
| | | | | | | | | | | | | | documenting PD |
| | | | | | | | | | | | | | is older than |
| | | | | | | | | | | | | | 28 days before |
| | | | | | | | | | | | | | entry into Second |
| | | | | | | | | | | | | | Course, a new |
| | | | | | | | | | | | | | image must be |
| | | | | | | | | | | | | | obtained and |
| | | | | | | | | | | | | | reviewed by the |
| | | | | | | | | | | | | | site before |
| | | | | | | | | | | | | | treatment. |
| | | | | | | | | | | | | | Perform imaging |
| | | | | | | | | | | | | | Q9W (63 days ± |
| | | | | | | | | | | | | | 7 days) through |
| | | | | | | | | | | | | | EOT, and Q12W |
| | | | | | | | | | | | | | (84 days ± 7 days) |
| | | | | | | | | | | | | | thereafter until PD |
| | | | | | | | | | | | | | is identified by the |
| | | | | | | | | | | | | | investigator. |
| | | | | | | | | | | | | | If imaging was |
| | | | | | | | | | | | | | obtained within |
| | | | | | | | | | | | | | 4 weeks before |
| | | | | | | | | | | | | | treatment DC, |
| | | | | | | | | | | | | | scan at DC is not |
| | | | | | | | | | | | | | mandatory. |
| | | | | | | | | | | | | | Follow-up visits |
| | | | | | | | | | | | | | may be scheduled |
| | | | | | | | | | | | | | to coincide with |
| | | | | | | | | | | | | | the imaging |
| | | | | | | | | | | | | | schedule. |
| Clinical Procedures/Assessments | | | | | | | | | | | | | If lenvatinib is |
| | | | | | | | | | | | | | DC'd, lenvatinib- |
| | | | | | | | | | | | | | specific |
| | | | | | | | | | | | | | procedures and |
| | | | | | | | | | | | | | assessments are |
| | | | | | | | | | | | | | only required at |
| | | | | | | | | | | | | | the EOT and |
| | | | | | | | | | | | | | Safety FU visits. |
| AE/SAE | X | X | X | X | X | X | X | X | X | X | X | X | Report AEs |
| monitoring | | | | | | | | | | | | | occurring within |
| | | | | | | | | | | | | | 30 days after the |
| | | | | | | | | | | | | | last dose of study |
| | | | | | | | | | | | | | intervention. |
| | | | | | | | | | | | | | Report SAEs |
| | | | | | | | | | | | | | occurring within |
| | | | | | | | | | | | | | 90 days after the |
| | | | | | | | | | | | | | last dose of study |
| | | | | | | | | | | | | | intervention, or 30 |
| | | | | | | | | | | | | | days after the last |
| | | | | | | | | | | | | | dose of study |
| | | | | | | | | | | | | | intervention if a |
| | | | | | | | | | | | | | new anticancer |
| | | | | | | | | | | | | | therapy is |
| | | | | | | | | | | | | | initiated, |
| | | | | | | | | | | | | | whichever is |
| | | | | | | | | | | | | | earlier. |
| Complete physical | X | | | | | | | | | X | | | An oral |
| examination | | | | | | | | | | | | | examination must |
| Directed physical | | X* | X | X | | X | X | X | X | | X | | be included. |
| examination | | | | | | | | | | | | | *Not required if a |
| | | | | | | | | | | | | | home virtual visit |
| | | | | | | | | | | | | | is performed. |
| Vital sign | X | X* | X | X | | X | X | X | X | X | X | | Predose and as |
| measurements | | | | | | | | | | | | | clinically |
| (including BP), | | | | | | | | | | | | | indicated. |
| weight | | | | | | | | | | | | | Participants with |
| | | | | | | | | | | | | | initial or recurrent |
| | | | | | | | | | | | | | systolic BP ≥ |
| | | | | | | | | | | | | | 160 mm Hg or |
| | | | | | | | | | | | | | diastolic BP ≥ |
| | | | | | | | | | | | | | 100 mm Hg must |
| | | | | | | | | | | | | | have their BP |
| | | | | | | | | | | | | | monitored until |
| | | | | | | | | | | | | | systolic BP |
| | | | | | | | | | | | | | is ≤150 mm Hg and |
| | | | | | | | | | | | | | diastolic BP |
| | | | | | | | | | | | | | is ≤95 mm Hg for |
| | | | | | | | | | | | | | 2 consecutive |
| | | | | | | | | | | | | | treatment cycles. |
| | | | | | | | | | | | | | *A home virtual |
| | | | | | | | | | | | | | visit can be |
| | | | | | | | | | | | | | scheduled to |
| | | | | | | | | | | | | | report BP and |
| | | | | | | | | | | | | | AEs. |
| 12-lead ECG | X | | | X | | | | | X | X | X | | After C2D1, ECG |
| | | | | | | | | | | | | | to be performed |
| | | | | | | | | | | | | | D1 of every 4th |
| | | | | | | | | | | | | | cycle (12 weeks) |
| | | | | | | | | | | | | | thereafter (e.g., |
| | | | | | | | | | | | | | C6, C10, C14, |
| | | | | | | | | | | | | | etc.). |
| | | | | | | | | | | | | | ECG at C1D1 and |
| | | | | | | | | | | | | | C2D1 should be |
| | | | | | | | | | | | | | performed |
| | | | | | | | | | | | | | approximately 2 |
| | | | | | | | | | | | | | hours post- |
| | | | | | | | | | | | | | lenvatinib dose. |
| | | | | | | | | | | | | | For high-risk |
| | | | | | | | | | | | | | patients, conduct |
| | | | | | | | | | | | | | ECG monitoring |
| | | | | | | | | | | | | | every cycle. |
| | | | | | | | | | | | | | If lenvatinib is |
| | | | | | | | | | | | | | discontinued, |
| | | | | | | | | | | | | | ECGs are only |
| | | | | | | | | | | | | | required at the |
| | | | | | | | | | | | | | EOT and Safety |
| | | | | | | | | | | | | | Follow-up visits. |
| LVEF assessment | X | | | | | | | | | | X | | Additional |
| by MUGA or | | | | | | | | | | | | | assessments as |
| ECHO | | | | | | | | | | | | | clinically |
| | | | | | | | | | | | | | indicated. |
| | | | | | | | | | | | | | Assessments |
| | | | | | | | | | | | | | should use the |
| | | | | | | | | | | | | | same method |
| | | | | | | | | | | | | | (MUGA or |
| | | | | | | | | | | | | | ECHO) |
| | | | | | | | | | | | | | throughout the |
| | | | | | | | | | | | | | study. |
| ECOG | X | | | X | | X | X | X | X | X | X | | Performed within |
| performance status | | | | | | | | | | | | | 7 days of |
| | | | | | | | | | | | | | beginning of C1 |
| | | | | | | | | | | | | | and before each |
| | | | | | | | | | | | | | treatment |
| | | | | | | | | | | | | | administration. |

Laboratory Procedures/Assessment

| | | | | | | | | | | |
|---|---|---|---|---|---|---|---|---|---|---|
| Pregnancy test for | X | | X | X | X | X | X | X | X | WOCBP require |
| WOCBP | | | | | | | | | | negative serum |
| | | | | | | | | | | test within 72 |
| | | | | | | | | | | hours or negative |
| | | | | | | | | | | urine test within |
| | | | | | | | | | | 24 hours before |
| | | | | | | | | | | each dose of study |
| | | | | | | | | | | intervention. |
| | | | | | | | | | | Additional testing |
| | | | | | | | | | | to be performed as |
| | | | | | | | | | | clinically |
| | | | | | | | | | | indicated. |
| CBC with | X | X | X | X | X | X | X | X | X | After C1, collect |
| differential | | | | | | | | | | within 3 days |
| Chemistry Panel | X | X | X | X | X | X | X | X | X | before dosing. |
| Urinalysis | X | | | | | X | X | X | X | Urinalysis should |
| | | | | | | | | | | be performed |
| | | | | | | | | | | every 4 cycles |
| | | | | | | | | | | (C5, C9, etc.). |
| Urine dipstick | X | | X | X | X | X | X | X | X | Note: urinalysis |
| testing for protein | | | | | | | | | | may be used if the |
| | | | | | | | | | | urine dipstick is |
| | | | | | | | | | | not feasible. |
| PT/INR and | X | | | | | | | | | Additional testing |
| aPTT/PTT | | | | | | | | | | to be conducted as |
| | | | | | | | | | | clinically |
| | | | | | | | | | | indicated for |
| | | | | | | | | | | participants taking |
| | | | | | | | | | | anticoagulant |
| | | | | | | | | | | therapy. |
| Thyroid function | X | | X | | X | | X | X | X | Should be |
| (T3, T4, and TSH) | | | | | | | | | | performed at C1 |
| | | | | | | | | | | and predose of |
| | | | | | | | | | | every other cycle |
| | | | | | | | | | | starting from C2 |
| | | | | | | | | | | (i.e., C4, C6, C8 |
| | | | | | | | | | | etc.). |
| | | | | | | | | | | Participants may |
| | | | | | | | | | | be dosed in |
| | | | | | | | | | | subsequent cycles |
| | | | | | | | | | | after C1 while |
| | | | | | | | | | | thyroid function |
| | | | | | | | | | | tests are pending. |
| | | | | | | | | | | Free T3 and free |
| | | | | | | | | | | T4 are acceptable. |

Abbreviations: AE = adverse event; aPTT = activated partial thromboplastin time; BP = blood pressure; BW = body weight; C# = cycle number; CBC = complete blood count; CR = complete response; CT = computed tomography; D# = day number; DC = discontinuation; ECG = electrocardiogram; ECHO = echocardiogram; ECOG = Eastern Cooperative Oncology Group; EOT = end-of-treatment; INR = international normalized ratio; IV = intravenous(ly); LVEF = left ventricular ejection fraction; MRI = magnetic resonance imaging; MUGA = multiple-gated acquisition; NYHA = New York Heart Association; PD = progressive disease; PT = prothrombin time; PTT = partial thromboplastin time; Q3W = every 3 weeks; Q9W = every 9 weeks; Q12W = every 12 weeks; qd = once daily; SAE = serious adverse event; SOC = standard of care; T3 = triiodothyronine; T4 = thyroxine; TSH = thyroid stimulating hormone; UPCR = urine protein-to-creatinine ratio; WOCBP = women of childbearing potential.

### Example 2

The present Example describes anti-tumor effects of a combination of a TIGIT antagonist, a PD-1 antagonist and lenvatinib using a CT26 tumor model.

Prior to treatment initiation, female BALB/c mice aged 8 weeks weighing between 18 to 21 grams were anesthetized and subcutaneously injected into the rear flank with 0.3×10⁶ CT26 log-phase sub-confluent cells. When the mean tumor volume of inoculated animals with CT26 reached approximately 146 mm³ (13 days later) mice were pair-matched into 8 treatment groups consisting of 10 mice per group.

Antibody Reagents in this Example Include:
Anti-PD-1 mIgG1 antibody, which is a murinized version of a rat anti-mouse PDT Ab with a mouse IgG1 Fe;
Anti-TIGIT mIgG2a antibody having a mouse Fe region of mIgG2a subtype that is the murine counterpart isotype to human isotype IgG1;
Isotype mIgG1 antibody, which is a mouse IgG1 isotype-matched control monoclonal antibody specific for adenoviral hexon 25; and
Isotype mIgG2a antibody, which is a mouse IgG2a isotype-matched control monoclonal antibody specific for adenoviral hexon 25.

The amino acid sequences of antibody reagents are listed in Table 16.

TABLE 16
Sequences of antibodies used in Example 2.

| | | SEQ |
|---|---|---|
| | | ID |
| | Sequences | NO. |
| Isotype | QVQLQQPGAELVRPGSSVKLSCKASGYTFNHYWIH | 296 |
| mIgG1 | WVKQGPLQGLEWIGSIDPSDSEVNYNHKFRDKATL | |
| antibody- | TVDKSSSTAYMHLSSLTSEDSAVYYCAKGLRDSWG | |
| heavy | QGTTLTVSSAKTTPPSVYPLAPGSAAQTNSMVTLG | |
| chain | CLVKGYFPEPVTVTWNSGSLSSGVHTFPAVLQSDL | |
| | YTLSSSVTVPSSTWPSETVTCNVAHPASSTKVDKK | |
| | IVPRDCGCKPCICTVPEVSSVFIFPPKPKDVLTIT | |
| | LTPKVTCVVVAISKDDPEVQFSWFVDDVEVHTAQT | |
| | QPREEQFNSTFRSVSELPIMHQDWLNGKEFKCRVN | |
| | SAAFPAPIEKTISKTKGRPKAPQVYTIPPPKEQMA | |
| | KDKVSLTCMITDFFPEDITVEWQWNGQPAENYKNT | |
| | QPIMDTDGSYFVYSKLNVQKSNWEAGNTFTCSVLH | |
| | EGLHNHHTEKSLSHSPGK | |
| Isotype | QIVLTQSPAIMSASLGEEVTLTCSATSSVNFMHWY | 297 |
| mIgG1 | QQKSGTSPKLLIYSTSILASGVPSRFSGSGSGTFY | |
| antibody- | SLTISSVEAEDAADYYCHQWTSYPWTFGGGTKLEI | |
| light | KRADAAPTVSIFPPSSEQLTSGGASVVCFLNNFYP | |
| chain | KDINVKWKIDGSERQNGVLNSWTDQDSKDSTYSMS | |
| | STLTLTKDEYERHNSYTCEATHKTSTSPIVKSFNR | |
| | NEC | |
| Isotype | QVQLQQPGAELVRPGSSVKLSCKASGYTFNHYWIH | 298 |
| mIgG2a | WVKQGPLQGLEWIGSIDPSDSEVNYNHKFRDKATL | |
| antibody- | TVDKSSSTAYMHLSSLTSEDSAVYYCAKGLRDSWG | |
| heavy | QGTTLTVSSAKTTAPSVYPLAPVCGDTTGSSVTLG | |
| chain | CLVKGYFPEPVTLTWNSGSLSSGVHTFPAVLQSDL | |
| | YTLSSSVTVTSSTWPSQSITCNVAHPASSTKVDKK | |
| | IEPRGPTIKPCPPCKCPAPNLLGGPSVFIFPPKIK | |
| | DVLMISLSPIVTCVVVDVSEDDPDVQISWFVNNVE | |
| | VHTAQTQTHREDYNSTLRVVSALPIQHQDWMSGKE | |
| | FKCKVNNKDLPAPIERTISKPKGSVRAPQVYVLPP | |
| | PEEEMTKKQVTLTCMVTDFMPEDIYVEWTNNGKTE | |
| | LNYKNTEPVLDSDGSYFMYSKLRVEKKNWVERNSY | |
| | SCSVVHEGLHNHHTTKSFSRTPGK | |
| Isotype | QIVLTQSPAIMSASLGEEVTLTCSATSSVNFMHWY | 299 |
| mIgG2a | QQKSGTSPKLLIYSTSILASGVPSRFSGSGSGTFY | |
| antibody- | SLTISSVEAEDAADYYCHQWTSYPWTFGGGTKLEI | |
| light | KRADAAPTVSIFPPSSEQLTSGGASVVCFLNNFYP | |
| chain | KDINVKWKIDGSERQNGVLNSWTDQDSKDSTYSMS | |
| | STLTLTKDEYERHNSYTCEATHKTSTSPIVKSFNR | |
| | NEC | |
| Anti-PD-1 | EVQLVESGGGLVQPGGSLKLSCAASGFTFSNSGLA | 300 |
| mIgG1 | WVRQAPEKGLEWVATITYNGTSTYYRDSVKGRFTI | |
| antibody- | SRDNAKNTLYLQMSSLRSEDTATYYCARWVPGSGN | |
| heavy | FDYWGQGTLVTVSSAKTTPPSVYPLAPGSAAQTNS | |
| chain | MVTLGCLVKGYFPEPVTVTWNSGSLSSGVHTFPAV | |
| | LQSDLYTLSSSVTVPSSTWPSETVTCNVAHPASST | |
| | KVDKKIVPRDCGCKPCICTVPEVSSVFIFPPKPKD | |
| | VLTITLTPKVTCVVVAISKDDPEVQFSWFVDDVEV | |
| | HTAQTQPREEQFNSTFRSVSELPIMHQDWLNGKEF | |
| | KCRVNSAAFPAPIEKTISKTKGRPKAPQVYTIPPP | |
| | KEQMAKDKVSLTCMITDFFPEDITVEWQWNGQPAE | |
| | NYKNTQPIMDTDGSYFVYSKLNVQKSNWEAGNTFT | |
| | CSVLHEGLHNHHTEKSLSHSPGK | |
| Anti-PD-1 | DIVLTQSPASLAVSLGQRATISCRASQSVTISRYT | 301 |
| mIgG1 | LMHWYQQKPGQPPKLLIYRASNLASGIPARFSGSG | |
| antibody- | SGTDFTLNIHPVEEDDAATYYCQQSRESPWTFGGG | |
| light | TKLEIKRADAAPTVSIFPPSSEQLTSGGASVVCFL | |
| chain | NNFYPKDINVKWKIDGSERQNGVLNSWTDQDSKDS | |
| | TYSMSSTLTLTKDEYERHNSYTCEATHKTSTSPIV | |
| | KSFNRNEC | |
| Anti-TIGIT | QVQLMESGPGLVQPSQTLSLTCTVSGFPLTSYTVH | 302 |
| mIgG2a | WVRQPPGKGLEWIGAIWSSGSTDYNSALKSRLNIN | |
| antibody- | RDSSKSQVFLKMNSLQTEDTAIYFCTKSGWAFFDY | |
| heavy | WGQGVMVTVSSAKTTAPSVYPLAPVCGDTTGSSVT | |
| chain | LGCLVKGYFPEPVTLTWNSGSLSSGVHTFPAVLQS | |
| | DLYTLSSSVTVTSSTWPSQSITCNVAHPASSTKVD | |
| | KKIEPRGPTIKPCPPCKCPAPNLLGGPSVFIFPPK | |
| | IKDVLMISLSPIVTCVVVDVSEDDPDVQISWFVNN | |
| | VEVHTAQTQTHREDYNSTLRVVSALPIQHQDWMSG | |
| | KEFKCKVNNKDLPAPIERTISKPKGSVRAPQVYVL | |
| | PPPEEEMTKKQVTLTCMVTDFMPEDIYVEWTNNGK | |
| | TELNYKNTEPVLDSDGSYFMYSKLRVEKKNWVERN | |
| | SYSCSVVHEGLHNHHTTKSFSRTPGK | |
| Anti-TIGIT | DIQMTQSPSLLSASVGDRVTLNCIASQNIYKSLAW | 303 |
| mIgG2a | YQLKLGEAPKLLIYNANSLQAGIPSRFSGSGSGTD | |
| antibody- | FALTISGLQPEDVATYFCQQYSGGYTFGAGTKLEL | |
| light | KRADAAPTVSIFPPSSEQLTSGGASVVCFLNNFYP | |
| chain | KDINVKWKIDGSERQNGVLNSWTDQDSKDSTYSMS | |
| | STLTLTKDEYERHNSYTCEATHKTSTSPIVKSFNR | |
| | NEC | |

### Treatment Groups are:

1) 0.5% methylcellulose (Vehicle)+isotype mIgG1 antibody+isotype mIgG2a antibody;
2) Vehicle+anti-PD-1 mIgG1 antibody+isotype mIgG1 antibody;
3) Vehicle+isotype mIgG1 antibody+anti-TIGIT mIgG2a antibody;
4) Lenvatinib+isotype mIgG1 antibody+isotype mIgG2a antibody;
5) Vehicle+anti-PD-1 mIgG1 antibody+anti-TIGIT mIgG2a antibody;
6) Lenvatinib+anti-PD-1 mIgG1 antibody+isotype mIgG2a antibody;
7) Lenvatinib+isotype mIgG1 antibody+anti-TIGIT mIgG2a antibody; and
8) Lenvatinib+anti-PD-1 mIgG1 antibody+anti-TIGIT mIgG2a antibody.

Vehicle and lenvatinib were orally gavage-dosed once daily (QD) at 10 mg/kg body weight. Isotype control, a mouse monoclonal antibody specific for adenoviral hexon on IgG1 or IgG2a backbones, as well as anti-PD-1 and anti-TIGIT antibodies were dosed intraperitoneally every 5 days at 10 mg/kg body weight. Start of treatments was considered Day 0 and dosing based on described schedules continued as described until Day 40. Caliper measurements of tumors and body weights were captured twice weekly. Statistical analyses of tumor growth inhibition (TGI) were performed by student t-test comparing treatment group to Vehicle group at Day 12. After this timepoint multiple animals from Vehicle group had reached endpoints and exited study. Other group comparisons were made on surviving animals at the end of study (Day 40) using a 1-way ANOVA with Tukey's multiple comparisons.

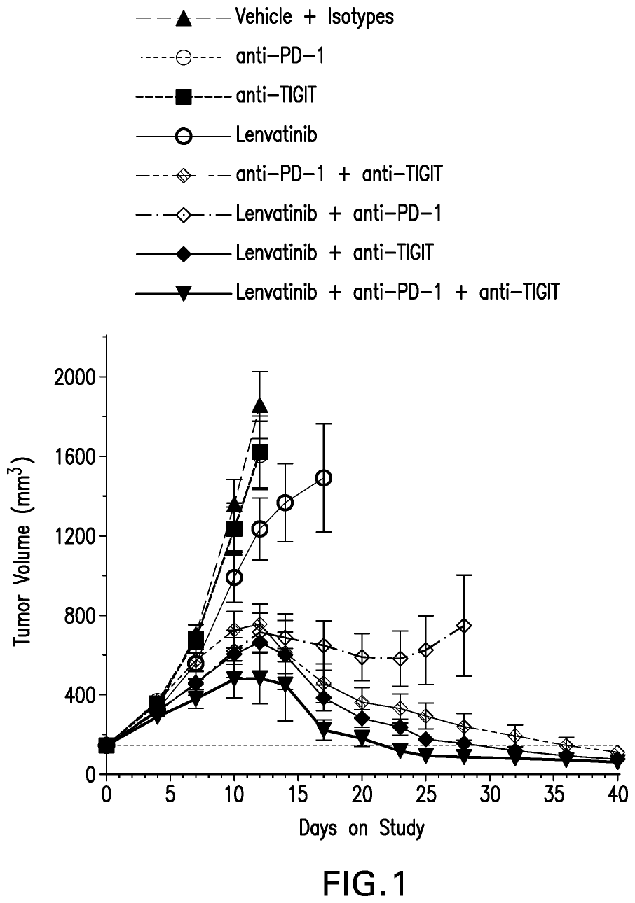

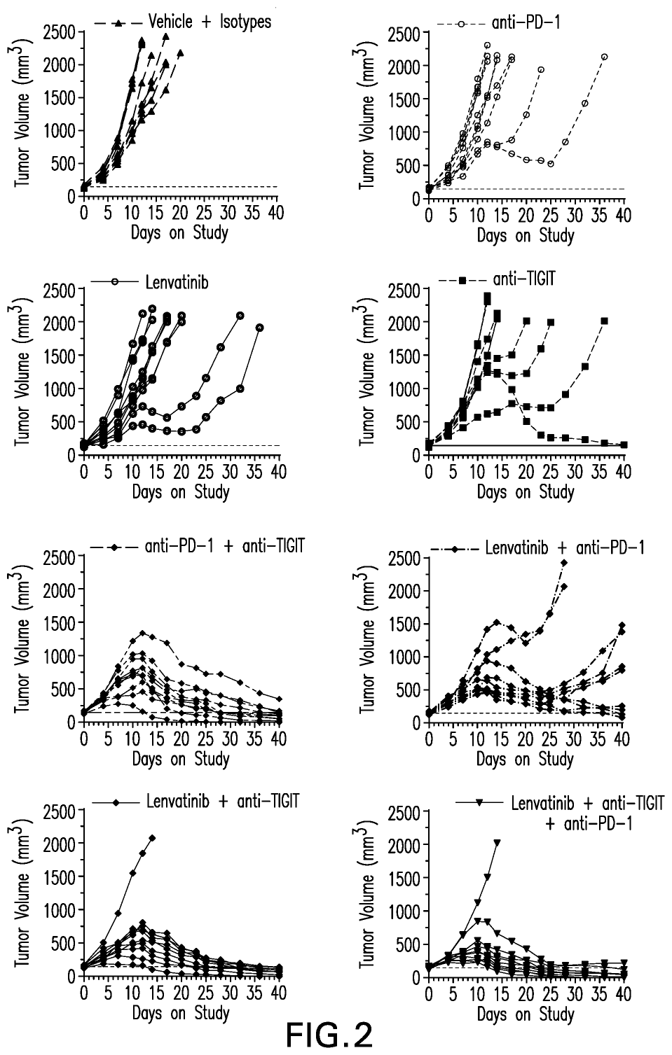

As shown in FIGS. 1 and 2 and Table 16, each monotherapy had subtle anti-tumor efficacy in the CT26 colorectal model when treatments started at 146 mm³ size. The dual therapies had robust anti-tumor efficacy and prolonged survival of most animals beyond Day 25 of treatment. Each dual combination exhibited better tumor growth inhibition than the respective monotherapies. At the end of the study on Day 40, a number of complete tumor regressions (CRs) and partial tumor regressions (PRs) were noted in multiple groups, as shown in Table 16. To compare the efficacy of groups reaching the end of study, 1-way ANOVA was performed, as summarized in Table 17. The dual combination of the anti-PD-1 antibody and the anti-TIGIT antibody and the triple combination of lenvatinib, the anti-PD-1 antibody, and the anti-TIGIT antibody were improved over the dual combination of lenvatinib and the anti-PD-1 antibody. Additionally, the dual combination of lenvatinib and the anti-TIGIT antibody was improved over the dual combination of lenvatinib and the anti-PD-1 antibody in terms of tumor growth inhibition and numbers of CRs and PRs, as shown in FIGS. 1 and 2 and Table 17. This data shows that the triple combination of lenvatinib, the anti-PD-1 antibody and the anti-TIGIT antibody improved upon the dual combination of lenvatinib and the anti-PD-1 antibody.

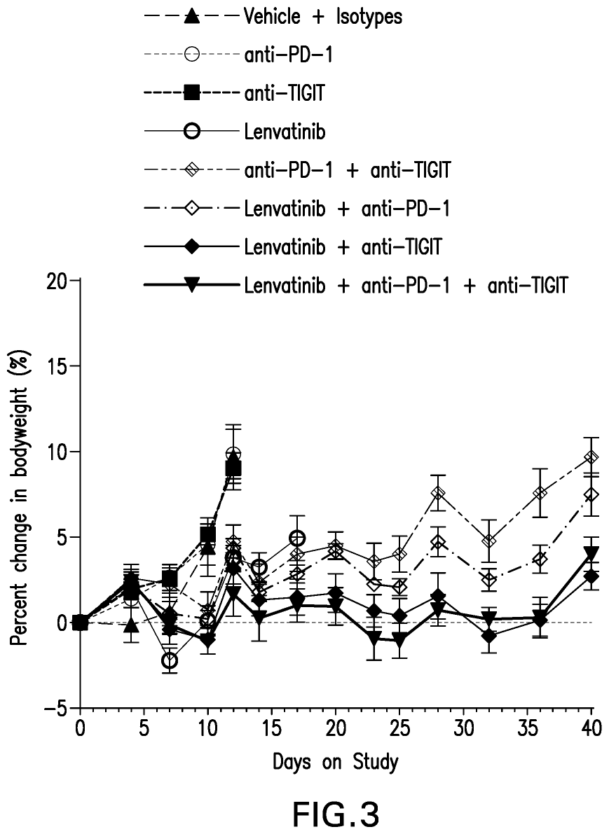

As assessed by body weight gain (FIG. 3), early mortality, and clinical observations, no adverse events were noted, thus all treatment regimens were determined to be well-tolerated by mice.

TABLE 16

| | TGI at Day | End of study |
|---|---|---|
| Treatment | 12 (p-value) | CRs/PRs |
| anti-PD-1 antibody monotherapy | 15% (p = 0.309) | 0 CRs, 0 PRs |
| anti-TIGIT antibody monotherapy | 14% (p = 0.355) | 0 CRs, 1 PR |
| Lenvatinib monotherapy | 36% (p = 0.014) | 0 CRs, 0 PRs |
| Anti-PD-1 antibody + anti-TIGIT | 64% (p < 0.001) | 2 CRs, 6 PRs |
| antibody | | |
| Lenvatinib + anti-PD-1 antibody | 67% (p < 0.001) | 0 CRs, 2 PRs |
| Lenvatinib + anti-TIGIT antibody | 70% (p < 0.001) | 2 CRs, 6 PRs |
| Lenvatinib + anti-PD-1 antibody + | 80% (p < 0.001) | 3 CRs, 5 PRs |
| anti-TIGIT antibody | | |

TABLE 17

| 1-way ANOVA at Day 40 | p-value |
|---|---|
| anti-PD-1 antibody + anti-TIGIT antibody | p = 0.001 |
| vs Lenvatinib + anti-PD-1 | |
| antibody | |
| anti-PD-1 antibody + anti-TIGIT antibody | p = 0.992 |
| vs Lenvatinib + anti-TIGIT | |
| antibody | |
| anti-PD-1 antibody + anti-TIGIT antibody | p = 0.978 |
| vs Triple Combo | |
| Lenvatinib + anti-TIGIT antibody vs | p < 0.001 |
| Lenvatinib + anti-PD-1 antibody | |
| Lenvatinib + anti-PD-1 antibody vs Triple combo | p < 0.001 |
| Lenvatinib + anti-TIGIT vs Triple combo | p = 0.999 |

1. A method of treating cancer, comprising administering to a human patient in need thereof an effective amount of a therapeutic combination comprising:
(a) a TIGIT antagonist;
(b) a PD-1 antagonist, wherein the PD-1 antagonist is not atezolizumab; and
(c) lenvatinib represented by Formula (I), 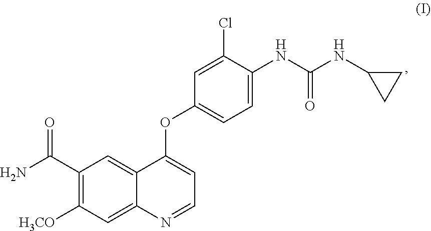 [SMILES: COc1cc2nccc(Oc3ccc(NC(=O)NC4CC4)c(Cl)c3)c2cc1C(N)=O]
or a pharmaceutically acceptable salt thereof.

2. The method of claim 1, wherein the cancer is selected from the group consisting of endometrial cancer, hepatocellular carcinoma (HCC), cervical cancer, head and neck cancer (HNSCC), biliary cancer, esophageal cancer, and triple negative breast cancer (TNBC).

3. The method of claim 2, wherein the cancer is HCC.

4. The method of claim 3, wherein the HCC is locally recurrent unresectable or metastatic.

5. (canceled)

6. The method of claim 2, wherein the cancer is endometrial cancer.

7. The method of claim 6, wherein the endometrial cancer is mismatch repair proficient.

8. A kit comprising:
(a) a TIGIT antagonist;
(b) a PD-1 antagonist, wherein the PD-1 antagonist is not atezolizumab; and
(c) lenvatinib represented by Formula (I), 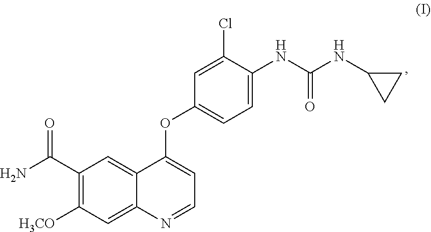 [SMILES: COc1cc2nccc(Oc3ccc(NC(=O)NC4CC4)c(Cl)c3)c2cc1C(N)=O]
or a pharmaceutically acceptable salt thereof.

9-16. (canceled)

17. The method of claim 1, wherein
i) the PD-1 antagonist and the TIGIT antagonist are co-formulated;
ii) the PD-1 antagonist and the TIGIT antagonist are in a fixed dose combination; or
iii) the PD-1 antagonist and the TIGIT antagonist are formulated separately.

18-20. (canceled)

21. The method of claim 17, wherein the anti-human PD-1 monoclonal antibody is a humanized antibody or human antibody.

22. (canceled)

23. The method of claim 1, wherein the TIGIT antagonist is an anti-human TIGIT monoclonal antibody or antigen binding fragment thereof.

24. The method of claim 23, wherein the anti-human TIGIT monoclonal antibody is a humanized antibody or human antibody.

25. (canceled)

26. The method of claim 17, wherein the anti-human PD-1 monoclonal antibody is pembrolizumab, nivolumab or cemiplimab.

27-28. (canceled)

29. The method of claim 23, wherein the anti-human TIGIT monoclonal antibody or antigen binding fragment thereof comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

30. The method of claim 29, wherein the anti-human TIGIT monoclonal antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152 and/or the anti-human TIGIT monoclonal antibody or antigen binding fragment thereof comprises a light chain comprising or consisting of the amino acid sequence as set forth in SEQ ID NO: 294 and a heavy chain comprising or consisting of the amino acid sequence as set forth in SEQ ID NO: 295.

31. (canceled)

32. The method of claim 1, wherein:
(a) the PD-1 antagonist is pembrolizumab; and
(b) the TIGIT antagonist comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

33. The method of claim 1, wherein:
(a) the PD-1 antagonist is nivolumab; and
(b) the TIGIT antagonist comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

34. The method of claim 1, wherein:
(a) the PD-1 antagonist is cemiplimab; and
(b) the TIGIT antagonist comprises three light chains CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

35. The method of claim 32, wherein the TIGIT antagonist comprises a heavy chain variable region comprising the amino acid sequence as set forth in SEQ ID NO: 148 and a light chain variable region comprising the amino acid sequence as set forth in SEQ ID NO: 152 and/or wherein the TIGIT antagonist comprises a light chain comprising or consisting of the amino acid sequence as set forth in SEQ ID NO: 294 and a heavy chain comprising or consisting of the amino acid sequence as set forth in SEQ ID NO: 295.

36. (canceled)

37. The method of claim 32, wherein the human patient is administered about 200 mg, about 240 mg, or about 2 mg/kg pembrolizumab, and wherein pembrolizumab is administered once every three weeks.

38-40. (canceled)

41. The method of claim 32, wherein the human patient is administered from about 100 mg to about 500 mg of the TIGIT antagonist, and wherein the TIGIT antagonist is administered once every three weeks.

42-44. (canceled)

45. The method of claim 1, wherein the human patient is administered about 4, about 8, about 10, about 12, about 14, about 18, about 20, or about 24 mg lenvatinib or a pharmaceutically acceptable salt thereof, and wherein lenvatinib or the pharmaceutically acceptable salt thereof is administered once daily.

46-51. (canceled)

52. The method of claim 1, wherein the pharmaceutically acceptable salt thereof is lenvatinib mesylate.

53. A method of treating endometrial cancer, comprising administering to a human patient in need thereof an effective amount of a therapeutic combination comprising:
(a) about 200 mg pembrolizumab;
(b) about 200 mg of an anti-TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110; and
(c) about 10 mg, about 14 mg, or about 20 mg lenvatinib or a pharmaceutically acceptable salt thereof.

54-59. (canceled)

60. A method of treating HCC, comprising administering to a human patient in need thereof an effective amount of a therapeutic combination comprising:
(a) about 200 mg pembrolizumab;
(b) about 200 mg of an anti-TIGIT antibody or antigen binding fragment thereof
comprising three light chain CDRs: comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110; and
(c) about 4 mg, about 8 mg, or about 12 mg lenvatinib or a pharmaceutically acceptable salt thereof.

61-67. (canceled)
